(19) United States
(12) Patent Application Publication
(10) Pub. No.: US 2009/0082555 A1
(43) Pub. Date: Mar. 26, 2009

(54) Aptamers to the human IL-12 cytokine family and their use as autoimmune disease therapeutics

(76) Inventors: John L. Diener, Cambridge, MA (US); Alicia Ferguson, Somerville, MA (US); Nobuko Hamaguchi, Framingham, MA (US); Sara Chesworth Keene, Tewksbury, MA (US); H.A. Daniel Lagasse, Somerville, MA (US); Pooja Sawhney, Arlington, MA (US); Kristin Thompson, Arlington, MA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/157,383

(22) Filed: Jun. 9, 2008

Related U.S. Application Data
(62) Division of application No. 11/075,649, filed on Mar. 7, 2005, now abandoned.
(60) Provisional application No. 60/550,962, filed on Mar. 5, 2004.
(60) Provisional application No. 60/608,046, filed on Sep. 7, 2004.

(51) Int. Cl. C12N 15/24

(52) U.S. Cl. 536/23.5

(57) ABSTRACT

The present invention provides materials and methods to treat immune disease in which cytokines are involved in pathogenesis. The materials and methods of the present invention are useful in the treatment of autoimmune diseases. The materials and methods of the present invention are directed to nucleic acid ligands capable of binding to human IL-23 and/or human IL-12 cytokines and thus modulate their biological activity and are useful as therapeutic agents in immune, auto-immune and cancer therapeutics.

### REFERENCE TO RELATED APPLICATIONS

This application is a divisional of U.S. patent application Ser. No. 11/075,649 filed on Mar. 7, 2005, which claims priority, under 35 U.S.C. § 119(e) to the following provisional applications: U.S. Provisional Patent Application Ser. No. 60/550,962, filed Mar. 5, 2004, and U.S. Provisional Patent Application Ser. No. 60/608,046, filed Sep. 7, 2004. Each of these applications are hereby incorporated by reference in their entireties.

### FIELD OF INVENTION

The invention relates generally to the field of nucleic acids and more particularly to aptamers capable of binding to members of the human interleukin-12 (IL-12) cytokine family, more specifically to human interleukin-12 (IL-12), human interleukin-23 (IL-23), or both IL-12 and IL-23, and to other related cytokines (e.g., IL-27 and p40 dimer). Such aptamers are useful as therapeutics in and diagnostics of autoimmune related diseases and/or other diseases or disorders in which the IL-12 family of cytokines, specifically IL-23 and IL-12, have been implicated. The invention further relates to materials and methods for the administration of aptamers capable of binding to IL-23 and/or IL-12.

### BACKGROUND OF THE INVENTION

Aptamers are nucleic acid molecules having specific binding affinity to molecules through interactions other than classic Watson-Crick base pairing.

Aptamers, like peptides generated by phage display or monoclonal antibodies (“mAbs”), are capable of specifically binding to selected targets and modulating the target's activity, e.g., through binding aptamers may block their target's ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarities, hydrophobic contacts, steric exclusion) that drive affinity and specificity in antibody-antigen complexes.

Aptamers have a number of desirable characteristics for use as therapeutics and diagnostics including high specificity and affinity, biological efficacy, and excellent pharmacokinetic properties. In addition, they offer specific competitive advantages over antibodies and other protein biologics, for example:

1) Speed and control. Aptamers are produced by an entirely in vitro process, allowing for the rapid generation of initial leads, including therapeutic leads. In vitro selection allows the specificity and affinity of the aptamer to be tightly controlled and allows the generation of leads, including leads against both toxic and non-immunogenic targets.

2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers most likely because aptamers cannot be presented by T-cells via the MHC and the immune response is generally trained not to recognize nucleic acid fragments.

3) Administration. Whereas most currently approved antibody therapeutics are administered by intravenous infusion (typically over 2-4 hours), aptamers can be administered by subcutaneous injection (aptamer bioavailability via subcutaneous administration is >80% in monkey studies (Tucker et al., J. Chromatography B. 732: 203-212, 1999)). This difference is primarily due to the comparatively low solubility and thus large volumes necessary for most therapeutic mAbs. With good solubility (>150 mg/mL) and comparatively low molecular weight (aptamer: 10-50 kDa; antibody: 150 kDa), a weekly dose of aptamer may be delivered by injection in a volume of less than 0.5 mL. In addition, the small size of aptamers allows them to penetrate into areas of conformational constrictions that do not allow for antibodies or antibody fragments to penetrate, presenting yet another advantage of aptamer-based therapeutics or prophylaxis.

4) Scalability and cost. Therapeutic aptamers are chemically synthesized and consequently can be readily scaled as needed to meet production demand. Whereas difficulties in scaling production are currently limiting the availability of some biologics and the capital cost of a large-scale protein production plant is enormous, a single large-scale oligonucleotide synthesizer can produce upwards of 100 kg/year and requires a relatively modest initial investment. The current cost of goods for aptamer synthesis at the kilogram scale is estimated at $500/g, comparable to that for highly optimized antibodies. Continuing improvements in process development are expected to lower the cost of goods to <$100/g in five years.

5) Stability. Therapeutic aptamers are chemically robust. They are intrinsically adapted to regain activity following exposure to factors such as heat and denaturants and can be stored for extended periods (>1 yr) at room temperature as lyophilized powders.

### Cytokines and the Immune Response

The immune response in mammals is based on a series of complex cellular interactions called the “immune network.” In addition to the network-like cellular interactions of lymphocytes, macrophages, granulocytes, and other cells, soluble proteins known as lymphokines, cytokines, or monokines play a critical role in controlling these cellular interactions. Cytokine expression by cells of the immune system plays an important role in the regulation of the immune response. Most cytokines are pleiotropic and have multiple biological activities including antigen-presentation; activation, proliferation, and differentiation of CD4+ cell subsets; antibody response by B cells; and manifestations of hypersensitivity. Cytokines are implicated in a wide range of degenerative or abnormal conditions which directly or indirectly involve the immune system and/or hematopoietic cells. An important family of cytokines is the IL-12 family which includes, e.g., IL-12, IL-23, IL-27, and p40 monomers and p40 dimers.

IL-23 is a covalently linked heterodimeric molecule composed of the p19 and p40 subunits, each encoded by separate genes. IL-12 is also a covalently linked heterodimeric molecule and consists of the p35 and p40 subunits. Thus, IL-23 and IL-12 both have the p40 subunit in common (FIG. 1). Human and mouse p19 share ˜70% amino acid sequence identity and are closely related to p35 (the subunit unique to IL-12). Transfection assays reveal that like p35, p19 protein is poorly secreted when expressed alone and requires the co-expression of its heterodimerizing partner p40 for higher expression. Together, p40 and p19 form a disulfide-linked heterodimer. The p19 component is produced in large amounts by activated macrophages, dendritic cells (“DCs”), endothelial cells, and T cells. Th1 cells express larger amounts of p19 mRNA than do Th2 cells; however, among these cell types only activated macrophages and DCs constitutively express p40, the other component of IL-23. The expression of p19 is increased by bacterial products that signal through the Toll-like receptor-2, which suggests that p19, and thus IL-23, may function in the immune response to certain bacterial infections.

One of the shared actions of IL-12 and IL-23 is their proliferative effect on T-cells (Brombacher et al., Trends in Immun. (2003)). However, clear differences exist in the T-cell subsets on which these cytokines act. In the mouse, IL-12 induces proliferation of naïve murine T cells but not memory T cells, whereas the proliferative effect of IL-23 is confined to memory T cells. In humans, IL-12 promotes proliferation of both naïve and memory human T-cells; however, the proliferative effect of IL-23 is still restricted to memory T cells. Also, the action of IL-23 on IFN-γ production is directed primarily toward memory T cells in humans. Although IL-12 can induce IFN-γ production in naïve T-cells and, to a greater extent, memory T-cells, IL-23 has very little effect on IFN-γ production in naïve T-cells. A moderate increase in IFN-γ production is observed in memory T-cells stimulated by IL-23, but this effect is somewhat smaller than that resulting from stimulation with IL-12.

Thus, IL-23 has biological activity that is distinct from IL-12, however both are believed to play a role in autoimmune and inflammatory diseases such as multiple sclerosis, rheumatoid arthritis, psoriasis, systemic lupus erythamatosus, and irritable bowel diseases (including Crohn's disease and ulcerative colitis), in addition to diseases such as bone resoprtion in osteoporosis, Type I Diabetes, and cancer.

### IL-23 and/or IL-12 Specific Aptamers as Autoimmune Disease Therapeutics

While not intending to be bound by theory, it is believed that IL-12 and IL-23 are involved in multiple sclerosis (“MS”) pathogenesis. For example, p40 levels are up-regulated in the cerebral spinal fluid of MS patients (Fassbender et al., (1998) Neurology 51:753). In addition, an anti-p40 mAb has been shown to localize to lesions in the brain (Brok et al., JI (2002)169:6554). Furthermore, lower baseline levels of p40 mRNA have been shown to predict clinical responsiveness to IFN-β treatment (Van-Boxel-Dezaire et al., 1999). Thus, a knock-down of both IL-12 and IL-23 via p40 might ameliorate the symptoms of MS. In fact, anti-p40 antibodies have been shown to significantly suppress the development and severity of Experimental Autoimmune Encephalomyelitis (“EAE”) in mice (Constantinescu et al., JI (1998) 161:5097) and in marmosets (Brok et al., JI (2002)169:6554).

Despite the evidence showing that knocking out both IL-23 and IL-12 suppresses the development and symptoms of MS, there is strong evidence that IL-23 is the more important of the two in MS/EAE pathogenesis in mice, as shown by the effects of IL-12 and IL-23 knock-outs on the EAE mouse model. (Cua et al., (2003) Nature 421:744). For example, EAE can occur in p35 knockout mice, but not p19 or p40 knock-out mice (Cua et al., (2003). Expression of IL-23 but not IL-12 in the CNS rescues EAE in p19/p40 knock-out mice, although over-expression of IL-12 exacerbates EAE, so IL-12 seems to play some role in general TH1 cell development and activation (Cua et al.). In humans, over-expression of p40 mRNA but not p35 mRNA has been observed in the Central Nervous System (CNS) of MS patients.

In addition to playing a general role in activating Th1 cells, IL-12 may be more important for fighting infection than IL-23. In mice, a p19 knock-out induces classic Th1 cell response (high IFN-gamma, low IL-4), whereas the response in p35 and p40 knock-out mice is restricted to Th2 cells (low IFN-gamma, high IL-4) (Cua et al.). Additionally, p19 knock-out immune cells produce strong pro-inflammatory cytokines, whereas p40 knock-out immune cells cannot. Lastly, p40, IL-12Rβ1 and IL-12Rβ2 knock-out mice are susceptible to a variety of infections (Adorini, from Contemporary Immunology (2003) pg. 253). Thus inhibiting IL-23 specifically through aptamer therapeutics may effectively fight IL-23 mediated disease while leaving the patient more able to fight infection.

Both IL-23 and/or IL-12 have been implicated in rheumatoid arthritis as a promoter of end-stage joint inflammation. While not intending to be bound by theory, it is believed that IL-23 affects the function of memory T-cells and inflammatory macrophages through engagement of the IL-23 receptor (IL-23R) on these cells. Studies indicate the IL-23 subunits p19 and/or p40 play a role in murine collagen-induced arthritis (“CIA”), the mouse model for rheumatoid arthritis. Anti-p40 antibodies have been shown to ameliorate the symptoms in murine CIA and prevent development and progression alone and when combined with anti-tumor necrosis factor (anti-TNF) treatment (Malfait et al., Clin. Exp. Immunol. (1998) 111:377, Matthys et al., Eur. J. Immunol. (1998) 28:2143, and Butler et al., Eur. J. Immunol. (1999) 29:2205). Furthermore, p19 and p40 knockout mice have been shown to be completely resistant to the development of CIA while CIA development and severity is exacerbated in p35 knock-out mice (McIntyre et al., Eur. J. Immunol. (1996) 26:2933, and Murphy et al., J. Exp. Med. (2003) 198:1951). Thus, the aptamers and methods of the present invention that bind to and inhibit IL-23 are useful as therapeutic agents for rheumatoid arthritis.

Both IL-23 and/or IL-12 are also believed to play a dominant role in the recruitment of inflammatory cells in Th-1 mediated diseases such as psoriasis vulgaris, and irritable bowel disease, including but not limited to Crohn's disease and ulcerative colitis. For example, elevated levels of p19 and p40 mRNA were detected by quantitative RT-PCR in skin lesions of patients with psoriasis vulgaris, whereas p35 mRNA was not (Lee et al., J Exp Med (2004) 199(1):125-30). In 2, 4, 6, trinitrobenzene sulfonic acid (“TNBS”) colitis, an experimental model of inflammatory bowel disease in mice, treatment with an anti-IL-12 monoclonal antibody proved efficacious in completely ameliorating/preventing mucosal inflammation (Neurath et al., J Exp Med (1995) 182:1281-1290). In another study which evaluated several different IL-12 antagonists in the TNBS colitis model, an anti-IL-12 p40 antibody proved to be the most effective in preventing mucosal inflammation, thus implicating both IL-12 and IL-23 (Schmidt et al., Pathobiology (2002-03); 70:177-183). Thus, the aptamers of the present invention that bind to and inhibit IL-12 and/or IL-23 are useful as therapeutic agents for psoriasis and inflammatory bowel diseases.

It is also believed that IL-12 and/or IL-23 play a role in systemic lupus erythamatosus (“SLE”). For example, serum obtained from SLE patients were found to contain significantly higher amounts of p40 as a monomer than serum levels of p40 as a heterodimer e.g., IL-12 (p35/p40) and IL-23 (p19/p40), indicating that deficient IL-23 and/or IL-12 production may play a role in the pathogenesis of SLE. Thus, aptamers of the invention which enhance the biological function of IL-23 and/or IL-12 are useful as therapeutics in the treatment of systemic lupus erythamatosus (Lauwerys et al., Lupus (2002) 11(6):384-7).

### IL-23 and/or IL-12 Specific Aptamers as Oncological Therapeutics

The anti-tumor activity of IL-12 has been well characterized, and recent studies have shown that IL-23 also possesses anti-tumor and anti-metastatic activity. For example, colon carcinoma cells retrovirally transduced with IL-23 significantly reduced the growth of colon tumors established by the cell line in immunocompetent mice as compared to a control cell line, indicating that the expression of IL-23 in tumors produces an anti-tumor effect. (Wang et al., Int. J. Cancer: 105, 820-824 (2003). Likewise, a lung carcinoma cell line retrovirally engineered to release single chain IL-23 (“scIL-23”) significantly suppressed lung metastases in BALB/c mice, resulting in almost complete tumor rejection (Lo et al., J. Immunol 2003, 171:600-607). Thus, aptamers that bind to IL-23 and/or IL-12 and enhance their biological function are useful as oncological therapeutics for the treatment of colon cancer, lung cancer, specifically lung metastases, and other oncological diseases for which IL-23 and/or IL-12 have an anti-tumor effect.

There is currently no known therapeutic agent that specifically targets human IL-23. Available agents that target IL-23 include an anti-human IL-23 p19 polyclonal antibody available through R&D Systems (Minneapolis, Minn.) for research use only, an anti-human p40 monoclonal antibody which targets both IL-12 and IL-23, since both cytokines have the p40 subunit in common, and anti-mouse IL-23 p19 polyclonal and monoclonal antibodies, which target mouse IL-23, not human IL-23 (Pirhonen, et al., (2002), J Immunology 169:5673-5678). As previously explained, an agent that inhibits the activity of both IL-23 and IL-12 may leave patients more vulnerable to infections, and generally can pose more complications in terms of developing a therapeutic agent than an agent that inhibits only IL-23. Since there is evidence that IL-23 plays a more important role than IL-12 for autoimmune inflammation in the brain and joints, a therapeutic specific for only IL-23 may be more advantageous than an agent which targets both cytokines, such as the anti-p40 human mAb.

Given the advantages of specificity, small size, and affinity of aptamers as therapeutic agents, it would be beneficial to have materials and methods for aptamer therapeutics to treat diseases in which human cytokines, specifically IL-23 and IL-12, play a role in pathogenesis. The present invention provides materials and methods to meet these and other needs.

### SUMMARY OF THE INVENTION

The present invention provides materials and methods for the treatment of autoimmune and inflammatory disease and other related diseases/disorders in which IL-23 and/or IL-12 are involved in pathogenesis.

In one embodiment, the materials of the present invention provide aptamers that specifically bind to IL-23. In one embodiment, IL-23 to which the aptamers of the invention bind is human IL-23 while in another embodiment IL-23 is a variant of human IL-23. In one embodiment the variant of IL-23 performs a biological function that is essentially the same as a function of human IL-23 and has substantially the same structure and substantially the same ability to bind said aptamer as that of human IL-23.

In one embodiment, human IL-23 or a variant thereof comprises an amino acid sequence which is at least 70% identical, preferably at least 80% identical, more preferably at least 90% identical to a sequence comprising SEQ ID NOs 4 and/or 5. In another embodiment, human IL-23 or a variant thereof has an amino acid sequence comprising SEQ ID NOs 4 and 5.

In one embodiment, the aptamer of the invention has a dissociation constant for human IL-23 or a variant thereof of about 100 nM or less, preferably 50 nM or less, more preferably 10 nM or less, even more preferably 1 nM or less.

In one embodiment, the aptamer of the present invention modulates a function of human IL-23 or a variant thereof. In one embodiment, the aptamer of the present invention stimulates a function of human IL-23. In another embodiment, the aptamer of the present invention inhibits a function of human IL-23 or a variant thereof. In yet another embodiment, the aptamer of the present invention inhibits a function of human IL-23 or a variant thereof in vivo. In yet another embodiment, the aptamer of the present invention prevents IL-23 from binding to the IL-23 receptor. In some embodiments, the function of human IL-23 or a variant thereof which is modulated by the aptamer of the invention is to mediate a disease associated with human IL-23 such as: autoimmune disease (including but not limited to multiple sclerosis, rheumatoid arthritis, psoriasis, systemic lupus erythamatosus, and irritable bowel disease (e.g., Crohn's Disease and ulcerative colitis)), inflammatory disease, cancer (including but not limited to colon cancer, lung cancer, and lung metastases), bone resorption in osteoporosis, and Type I Diabetes.

In one embodiment, the aptamer of the invention has substantially the same ability to bind human IL-23 as that of an aptamer comprising a nucleotide sequence selected from the group consisting of: SEQ ID NOs 13-66, SEQ ID NOs 71-88, SEQ ID NOs 91-96, SEQ ID NOs 103-118, SEQ ID NOs 124-130, SEQ ID NOs 1435-159, SEQ ID NO 162, and SEQ ID NOs 164-172, SEQ ID NOs 176-178, SEQ ID NOs 181-196, and SEQ ID NOs 203-314. In another embodiment the aptamer of the invention has substantially the same structure and substantially the same ability to bind IL-23 as that of an aptamer comprising a nucleotide sequence selected from the group consisting of: SEQ ID NOs 13-66, SEQ ID NOs 71-88, SEQ ID NOs 91-96, SEQ ID NOs 103-118, SEQ ID NOs 124-130, SEQ ID NOs 135-159, SEQ ID NO 162, and SEQ ID NOs 164-172, SEQ ID NOs 176-178, SEQ ID NOs 181-196, and SEQ ID NOs 203-314.

In one embodiment, the present invention provides an aptamer that binds to human IL-23 comprising a nucleic acid sequence at least 80% identical, more preferably at least 90% identical to any one of the sequences selected from the group consisting of: SEQ ID NOs 13-66, SEQ ID NOs 71-88, SEQ ID NOs 91-96, SEQ ID NOs 103-118, SEQ ID NOs 124-130, SEQ ID NOs 135-159, SEQ ID NO 162, and SEQ ID NOs 164-172, SEQ ID NOs 176-178, SEQ ID NOs 181-196, and SEQ ID NOs 203-314. In another embodiment, the present invention provides an aptamer comprising 4 contiguous nucleotides, preferably 8 contiguous nucleotides, more preferably 20 contiguous nucleotides that are identical to a sequence of 4, 8, or 20 contiguous nucleotides in the unique sequence region of any one of the sequences selected from the group of: SEQ ID NOs 13-66, SEQ ID NOs 71-88, SEQ ID NOs 91-96, SEQ ID NOs 103-118, SEQ ID NOs 124-130, SEQ ID NOs 135-159, SEQ ID NO 162, and SEQ ID NOs 164-172, SEQ ID NOs 176-178, SEQ ID NOs 181-196, and SEQ ID NOs 203-314. In yet another embodiment the present invention provides an aptamer capable of binding human IL-23 or a variant thereof comprising a nucleotide sequence selected from the group consisting of: SEQ ID NOs 13-66, SEQ ID NOs 71-88, SEQ ID NOs 91-96, SEQ ID NOs 103-118, SEQ ID NOs 124-130, SEQ ID NOs 135-159, SEQ ID NO 162, and SEQ ID NOs 164-172, SEQ ID NOs 176-178, SEQ ID NOs 181-196, and SEQ ID NOs 203-314. In another embodiment, the present invention provides an aptamer having the sequence set forth in SEQ ID NO 177, preferably SEQ ID NO 224, more preferably SEQ ID NO 309, more preferably SEQ ID NO 310, and more preferably SEQ ID NO 311.

In one embodiment, the present invention provides aptamers that specifically bind to mouse IL-23. In another embodiment, the present invention provides aptamers that bind to a variant of mouse IL-23 that performs a biological function that is essentially the same as a function of mouse IL-23 and has substantially the same structure and substantially the same ability to bind said aptamer as that of mouse IL-23.

In one embodiment, mouse IL-23 or a variant thereof to which the aptamer of the invention binds comprises an amino acid sequence which is at least 80%, preferably at least 90% identical to a sequence comprising SEQ ID NOs 315 and/or 316. In another embodiment mouse IL-23 or a variant thereof has an amino acid sequence comprising SEQ ID NOs 315 and 316.

In one embodiment, the aptamer of the invention has a dissociation constant for mouse IL-23 or a variant thereof of about 100 nM or less, preferably 50 nM or less, more preferably 10 nM or less.

In one embodiment, the aptamer of the invention modulates a function of mouse IL-23 or a variant thereof. In one embodiment, the aptamer of the invention stimulates a function of mouse IL-23. In another embodiment, the aptamer of the invention inhibits a function of mouse IL-23 or a variant thereof. In yet another embodiment, the aptamer of the invention inhibits a function of mouse IL-23 or a variant thereof in vivo. In yet another embodiment, the aptamer of the invention prevents the binding of mouse IL-23 to the mouse IL-23 receptor. In some embodiments, the function of mouse IL-23 which is modulated by the aptamer of the present invention is to mediate a disease model associated with mouse IL-23 such as experimental autoimmune encephalomyelitis, murine collagen-induced arthritis, and TNBS colitis.

In one embodiment, the aptamer of the invention has substantially the same ability to bind mouse IL-23 as that of an aptamer comprising a nucleotide sequence selected from the group consisting of SEQ ID NOs 124-134 and SEQ ID NOs 199-202. In another embodiment, the aptamer of the invention has substantially the same structure and substantially the same ability to bind mouse IL-23 as that of an aptamer comprising a nucleotide sequence selected from the group consisting of SEQ ID NOs 124-134 and SEQ ID NOs 199-202.

In one embodiment, the present invention provides aptamers that bind to mouse IL-23 comprising a nucleic acid sequence at least 80% identical, preferably at least 90% identical to any one of the sequences selected from the group consisting of SEQ ID NOs 124-134, and SEQ ID NOs 199-202. In another embodiment, the present invention provides aptamers comprising 4 contiguous, preferably 8 contiguous, more preferably 20 contiguous nucleotides that are identical to a sequence of 4, 8 or 20 contiguous nucleotides in the unique sequence region of any one of the sequences selected from the group consisting of: SEQ ID NOs 124-134 and SEQ ID NOs 199-202. In another embodiment, the present invention provides an aptamer capable of binding mouse IL-23 or a variant thereof comprising a nucleotide sequence selected from the group consisting of: SEQ ID NOs 124-134 and SEQ ID NOs 199-202.

In one embodiment, the materials of the present invention provide aptamers that specifically bind to IL-12. In one embodiment, IL-12 to which the aptamers of the invention bind is human IL-12 while in another embodiment IL-12 is a variant of human IL-12. In one embodiment the variant of IL-12 performs a biological function that is essentially the same as a function of human IL-12 and has substantially the same structure and substantially the same ability to bind said aptamer as that of human IL-12.

In one embodiment, human IL-12 or a variant thereof comprises an amino acid sequence which is at least 80% identical, preferably at least 90% identical to a sequence comprising SEQ ID NOs 4 and/or 6. In another embodiment, human IL-12 or a variant thereof has an amino acid sequence comprising SEQ ID NOs 4 and 6.

In one embodiment, the aptamer of the present invention modulates a function of human IL-12 or a variant thereof. In one embodiment, the aptamer of the present invention stimulates a function of human IL-23. In another embodiment, the aptamer of the present invention inhibits a function of human IL-12 or a variant thereof. In yet another embodiment, the aptamer of the present invention inhibits a function of human IL-12 or a variant thereof in vivo. In yet another embodiment, the aptamer of the present invention prevents IL-12 from binding to the IL-12 receptor. In one embodiment, the function of human IL-12 or a variant thereof which is modulated by the aptamer of the invention is to mediate a disease associated with human IL-12 such as: autoimmune disease (including but not limited to multiple sclerosis, rheumatoid arthritis, psoriasis, systemic lupus erythamatosus, and irritable bowel disease (e.g., Crohn's Disease and ulcerative colitis)), inflammatory disease, cancer (including but not limited to colon cancer, lung cancer, and lung metastases), bone resorption in osteoporosis, and Type I Diabetes.

In one embodiment, the present invention provides aptamers which are either ribonucleic or deoxyribonucleic acid. In a further embodiment, these ribonucleic or deoxyribonucleic acid aptamers are single stranded. In another embodiment, the present invention provides aptamers comprising at least one chemical modification. In one embodiment, the modification is selected from the group consisting of: a chemical substitution at a sugar position; a chemical substitution at a phosphate position; and a chemical substitution at a base position, of the nucleic acid; incorporation of a modified nucleotide; 3′ capping; conjugation to a high molecular weight, non-immunogenic compound; conjugation to a lipophilic compound; and phosphate backbone modification. In one embodiment, the non-immunogenic, high molecular weight compound conjugated to the aptamer of the invention is polyalkylene glycol, preferably polyethylene glycol. In one embodiment, the backbone modification comprises incorporation of one or more phosphorothioates into the phosphate backbone. In another embodiment, the aptamer of the invention comprises the incorporation of fewer than 10, fewer than 6, or fewer than 3 phosphorothioates in the phosphate backbone.

In one embodiment, the materials of the present invention provide a pharmaceutical composition comprising a therapeutically effective amount of an aptamer comprising a nucleic acid sequence selected from the group consisting of: SEQ ID NOs 13-66, SEQ ID NOs 71-88, SEQ ID NOs 91-96, SEQ ID NOs 103-118, SEQ ID NOs 124-130, SEQ ID NOs 135-159, SEQ ID NO 162, and SEQ ID NOs 164-172, SEQ ID NOs 176-178, SEQ ID NOs 181-196, and SEQ ID NOs 203-314, or a salt thereof, and a pharmaceutically acceptable carrier or diluent. In another embodiment, the materials of the present invention provide a pharmaceutical composition comprising a therapeutically effective amount of an aptamer comprising a nucleic acid sequence selected from the group consisting of: SEQ ID NO 14, SEQ ID NOs 17-19, SEQ ID NO 21, SEQ ID NOs 27-32, SEQ ID NOs 34-40, SEQ ID NO 42, SEQ ID NO 49, SEQ ID NOs 60-61, SEQ ID NOs 91-92, SEQ ID NO 94, and SEQ ID NOs 103-118, or a salt thereof, and a pharmaceutically acceptable carrier or diluent. In a preferred embodiment, the materials of the present invention provide a pharmaceutical composition comprising a therapeutically effective amount of an aptamer comprising a nucleic acid sequence selected from the group consisting of: SEQ ID NO 177, SEQ ID NO 224, and SEQ ID NOs 309-312.

In one embodiment, the present invention provides a method of treating, preventing or ameliorating a disease mediated by IL-23, comprising administering the composition comprising a therapeutically effective amount of an aptamer comprising a nucleic acid sequence selected from the group consisting of: SEQ ID NOs 13-66, SEQ ID NOs 71-88, SEQ ID NOs 91-96, SEQ ID NOs 103-118, SEQ ID NOs 124-130, SEQ ID NOs 135-159, SEQ ID NO 162, and SEQ ID NOs 164-172, SEQ ID NOs 176-178, SEQ ID NOs 181-196, and SEQ ID NOs 203-314, to a vertebrate. In another embodiment, the present invention provides a method of treating, preventing or ameliorating a disease mediated by IL-23 and/or IL-112, comprising administering the composition comprising a therapeutically effective amount of an aptamer comprising a nucleic acid sequence selected from the group consisting of: SEQ ID NO 14, SEQ ID NOs 17-19, SEQ ID NO 21, SEQ ID NOs 27-32, SEQ ID NOs 34-40, SEQ ID NO 42, SEQ ID NO 49, SEQ ID NOs 60-61, SEQ ID NOs 91-92, SEQ ID NO 94, and SEQ ID NOs 103-118, to a vertebrate. In a preferred embodiment the composition comprising a therapeutically effective amount of an aptamer administered to a vertebrate comprises a nucleic acid sequence selected from the group consisting of: SEQ ID NO 177, SEQ ID NO 224, and SEQ ID NOs 309-312. In one embodiment the vertebrate to which the pharmaceutical composition is administered is a mammal. In a preferred embodiment, the mammal is a human.

In one embodiment, the disease treated, prevented or ameliorated by the methods of the present invention is selected from the group consisting of: autoimmune disease (including but not limited to multiple sclerosis, rheumatoid arthritis, psoriasis, systemic lupus erythamatosus, and irritable bowel disease (e.g., Crohn's Disease and ulcerative colitis)), inflammatory disease, cancer (including but not limited to colon cancer, lung cancer, and lung metastases), bone resorption in osteoporosis, and Type I Diabetes.

In one embodiment, the present invention provides a diagnostic method comprising contacting an aptamer with a nucleic acid sequence selected from the group consisting of: SEQ ID NOs 13-66, SEQ ID NOs 71-88, SEQ ID NOs 91-96, SEQ ID NOs 103-118, SEQ ID NOs 124-134, SEQ ID NOs 135-159, SEQ ID NO 162, and SEQ ID NOs 164-172, SEQ ID NOs 176-178, SEQ ID NOs 181-196, and SEQ ID NOs 199-314 with a composition suspected of comprising IL-23 and/or IL-12 or a variant thereof, and detecting the presence or absence of IL-23 and/or IL-12 or a variant thereof.

In one embodiment, the present invention provides an aptamer with a nucleic acid sequence selected from the group consisting of: SEQ ID NOs 13-66, SEQ ID NOs 71-88, SEQ ID NOs 91-96, SEQ ID NOs 103-118, SEQ ID NOs 124-134, SEQ ID NOs 135-159, SEQ ID NO 162, and SEQ ID NOs 164-172, SEQ ID NOs 176-178, SEQ ID NOs 181-196, and SEQ ID NOs 199-314 for use as an in vitro diagnostic. In another embodiment, the present invention provides an aptamer with a nucleic acid sequence selected from the group consisting of: SEQ ID NOs 13-66, SEQ ID NOs 71-88, SEQ ID NOs 91-96, SEQ ID NOs 103-118, SEQ ID NOs 124-134, SEQ ID NOs 135-159, SEQ ID NO 162, and SEQ ID NOs 164-172, SEQ ID NOs 176-178, SEQ ID NOs 181-196, and SEQ ID NOs 199-314 for use as an in vivo diagnostic. In yet another embodiment, the present invention provides an aptamer with a nucleic acid sequence selected from the group consisting of: SEQ ID NOs 13-66, SEQ ID NOs 71-88, SEQ ID NOs 91-96, SEQ ID NOs 103-118, SEQ ID NOs 124-134, SEQ ID NOs 135-159, SEQ ID NO 162, and SEQ ID NOs 164-172, SEQ ID NOs 176-178, SEQ ID NOs 181-196, and SEQ ID NOs 199-314 for use in the treatment, prevention or amelioration of disease in vivo.

### BRIEF DESCRIPTION OF THE DRAWINGS

FIG. 1 is a schematic representation of the Interleukin-12 family of cytokines.

FIG. 2 is a schematic representation of the in vitro aptamer selection (SELEX™) process from pools of random sequence oligonucleotides.

FIG. 3 is a schematic of the in vitro selection scheme for selecting aptamers specific to IL-23 by including IL-12 in the negative selection step thereby eliminating sequences that recognize p40, the common subunit in both IL-12 and IL-23.

FIG. 4 is an illustration of a 40 kDa branched PEG.

FIG. 5 is an illustration of a 40 kDa branched PEG attached to the 5′ end of an aptamer.

FIG. 6 is an illustration depicting various PEGylation strategies representing standard mono-PEGylation, multiple PEGylation, and dimerization via PEGylation.

FIG. 7 is a graph showing binding of rRmY and rGmH pools to IL-23 after various rounds of selection.

FIG. 8A is a representative schematic of the sequence and predicted secondary structure configuration of a Type 1 IL-23 aptamers; FIG. 8B is a representative schematic of the sequences and predicted secondary structure configuration of several Type 2 IL-23 aptamers.

FIG. 9A is a schematic of the minimized aptamer sequences and predicted secondary structure configurations for Type 1 IL-23 aptamers; FIG. 9B is a schematic of the minimized aptamer sequences and predicted secondary structure configurations for Type 2 IL-23 aptamers.

FIG. 10 depicts the predicted G-Quartet structure for dRmY minimer ARC979 (SEQ ID NO 177).

FIG. 11 is a graph showing an increase of NMM fluorescence in ARC979 (SEQ ID NO 177), confirming that ARC979 adopts a G-quartet structure.

FIG. 12 is a graph of the ARC979 (SEQ ID NO 177) competition binding curve analyzed based on total [aptamer] bound using 50 nM IL-23.

FIG. 13 is a graph of the ARC979 (SEQ ID NO 177) competition binding curve analyzed based on [aptamer] bound using 250 nM IL-12.

FIG. 14 is a graph of the direct binding curves for ARC979 (SEQ ID NO 177) under two different binding reaction conditions (1×PBS (without Ca⁺⁺ or Mg⁺⁺) or 1× Dulbeccos PBS (with Ca⁺⁺ and Mg⁺⁺).

FIG. 15 is a graph of the direct binding curves for ARC979 (SEQ ID NO 177) phosphorothioate derivatives depicting that single phosphorothioate substitutions yield increased proportion binding to IL-23.

FIG. 16 is a graph of the competition binding curves for ARC979 (SEQ ID NO 177) phosphorothioate derivatives depicting that single phosphorothioate substitutions compete for IL-23 at a higher affinity that ARC979.

FIG. 17 is a graph of the direct binding curves for the ARC979 optimized derivatives ARC1624 (SEQ ID NO 310) and ARC1625 (SEQ ID NO 311), compared to the parent ARC979 (SEQ ID NO 177) aptamer (ARC895 is a negative control).

FIG. 18 is a graph depicting the plasma stability of ARC979 (SEQ ID NO 177) compared to optimized ARC979 derivative constructs.

FIG. 19 is a schematic representation of the TransAM™ assay used to measure STAT3 activity in lysates of PHA blast cells exposed to aptamers of the invention.

FIG. 20 is a flow diagram of the protocol used for the detection of IL-23 induced STAT3 phosphorylation in PHA blasts exposed to aptamers of the invention.

FIG. 21 is a representative graph showing the inhibitory effect of parental IL-23 aptamers of rRfY composition compared to their respective optimized clones on IL-23 induced STAT3 phosphorylation in PHA Blasts using the TransAM™ Assay.

FIG. 22 is a graph of the percent inhibition of IL-23 induced STAT3 phosphorylation by IL-23 aptamers of dRmY composition in the TransAM™ assay (ARC793 (SEQ ID NO 163) is a non-binding aptamer).

FIG. 23 is a graph of the percent inhibition of IL-23 induced STAT3 phosphorylation by parental IL-23 aptamers of dRrnY composition (ARC621 (SEQ ID NO 108), ARC627 (SEQ ID NO 110)) compared to their respective optimized clones (ARC979 (SEQ ID NO 177), ARC980 (SEQ ID NO 178), ARC982 (SEQ ID NO 180)) in the TransAM™ assay.

FIG. 24 is a percent inhibition graph of IL-23 induced STAT 3 phosphorylation by ARC979 (SEQ ID NO 177) and two optimized derivative clones of ARC979 (ARC1624 (SEQ ID NO 310) and ARC1625 (SEQ ID NO 311)) in the Pathscan® assay.

FIG. 25 is a graph comparing human and mouse IL-23 induced STAT3 activation in human PHA Blasts, measured by the TransAM™ assay.

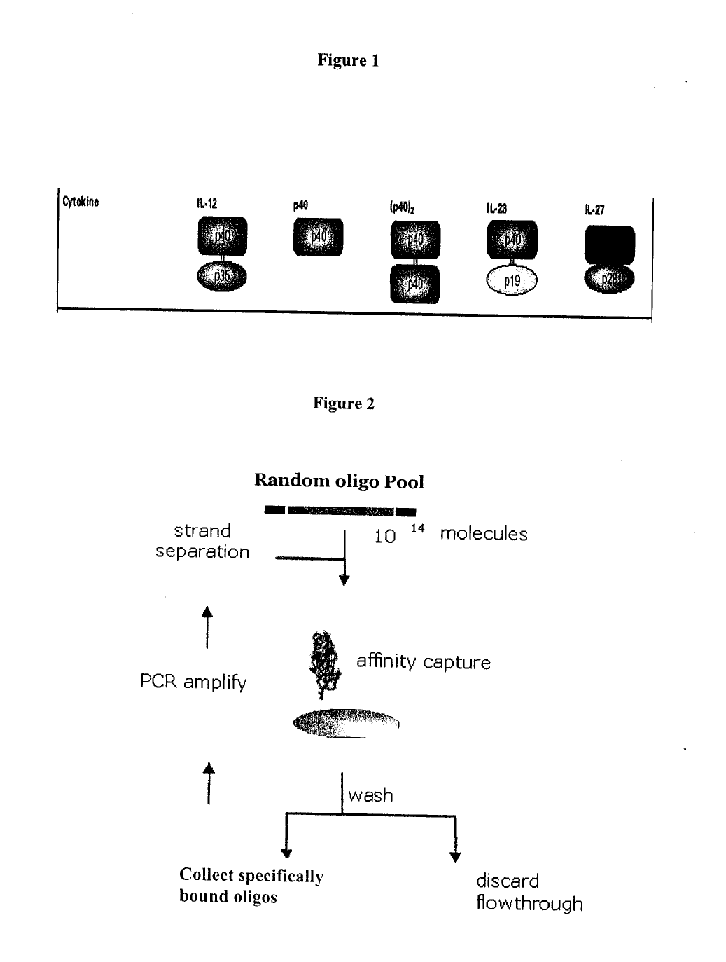

### DETAILED DESCRIPTION OF THE INVENTION

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

### The SELEX™ Method

A suitable method for generating an aptamer is with the process entitled “Systematic Evolution of Ligands by Exponential Enrichment” (“SELEX™”) generally depicted in FIG. 2. The SELEX™ process is a method for the in vitro evolution of nucleic acid molecules with highly specific binding to target molecules and is described in, e.g., U.S. patent application Ser. No. 07/536,428, filed Jun. 11, 1990, now abandoned, U.S. Pat. No. 5,475,096 entitled “Nucleic Acid Ligands”, and U.S. Pat. No. 5,270,163 (see also WO 91/19813) entitled “Nucleic Acid Ligands”. Each SELEX™-identified nucleic acid ligand, i.e., each aptamer, is a specific ligand of a given target compound or molecule. The SELEX™ process is based on the unique insight that nucleic acids have sufficient capacity for forming a variety of two- and three-dimensional structures and sufficient chemical versatility available within their monomers to act as ligands (i.e., form specific binding pairs) with virtually any chemical compound, whether monomeric or polymeric. Molecules of any size or composition can serve as targets.

SELEX™ relies as a starting point upon a large library or pool of single stranded oligonucleotides comprising randomized sequences. The oligonucleotides can be modified or unmodified DNA, RNA, or DNA/RNA hybrids. In some examples, the pool comprises 100% random or partially random oligonucleotides. In other examples, the pool comprises random or partially random oligonucleotides containing at least one fixed sequence and/or conserved sequence incorporated within randomized sequence. In other examples, the pool comprises random or partially random oligonucleotides containing at least one fixed sequence and/or conserved sequence at its 5′ and/or 3′ end which may comprise a sequence shared by all the molecules of the oligonucleotide pool. Fixed sequences are sequences common to oligonucleotides in the pool which are incorporated for a preselected purpose such as, CpG motifs described further below, hybridization sites for PCR primers, promoter sequences for RNA polymerases (e.g., T3, T4, T7, and SP6), restriction sites, or homopolymeric sequences, such as poly A or poly T tracts, catalytic cores, sites for selective binding to affinity columns, and other sequences to facilitate cloning and/or sequencing of an oligonucleotide of interest. Conserved sequences are sequences, other than the previously described fixed sequences, shared by a number of aptamers that bind to the same target.

The oligonucleotides of the pool preferably include a randomized sequence portion as well as fixed sequences necessary for efficient amplification. Typically the oligonucleotides of the starting pool contain fixed 5′ and 3′ terminal sequences which flank an internal region of 30-50 random nucleotides. The randomized nucleotides can be produced in a number of ways including chemical synthesis and size selection from randomly cleaved cellular nucleic acids. Sequence variation in test nucleic acids can also be introduced or increased by mutagenesis before or during the selection/amplification iterations.

The random sequence portion of the oligonucleotide can be of any length and can comprise ribonucleotides and/or deoxyribonucleotides and can include modified or non-natural nucleotides or nucleotide analogs. See, e.g., U.S. Pat. No. 5,958,691; U.S. Pat. No. 5,660,985; U.S. Pat. No. 5,958,691; U.S. Pat. No. 5,698,687; U.S. Pat. No. 5,817,635; U.S. Pat. No. 5,672,695, and PCT Publication WO 92/07065. Random oligonucleotides can be synthesized from phosphodiester-linked nucleotides using solid phase oligonucleotide synthesis techniques well known in the art. See, e.g., Froehler et al., Nucl. Acid Res. 14:5399-5467 (1986) and Froehler et al., Tet. Lett. 27:5575-5578 (1986). Random oligonucleotides can also be synthesized using solution phase methods such as triester synthesis methods. See, e.g., Sood et al., Nucl. Acid Res. 4:2557 (1977) and Hirose et al., Tet. Lett., 28:2449 (1978). Typical syntheses carried out on automated DNA synthesis equipment yield 10¹⁴-10¹⁶ individual molecules, a number sufficient for most SELEX™ experiments. Sufficiently large regions of random sequence in the sequence design increases the likelihood that each synthesized molecule is likely to represent a unique sequence.

The starting library of oligonucleotides may be generated by automated chemical synthesis on a DNA synthesizer. To synthesize randomized sequences, mixtures of all four nucleotides are added at each nucleotide addition step during the synthesis process, allowing for random incorporation of nucleotides. As stated above, in one embodiment, random oligonucleotides comprise entirely random sequences; however, in other embodiments, random oligonucleotides can comprise stretches of nonrandom or partially random sequences. Partially random sequences can be created by adding the four nucleotides in different molar ratios at each addition step.

The starting library of oligonucleotides may be either RNA or DNA. In those instances where an RNA library is to be used as the starting library it is typically generated by transcribing a DNA library in vitro using T7 RNA polymerase or modified T7 RNA polymerases and purified. The RNA or DNA library is then mixed with the target under conditions favorable for binding and subjected to step-wise iterations of binding, partitioning and amplification, using the same general selection scheme, to achieve virtually any desired criterion of binding affinity and selectivity. More specifically, starting with a mixture containing the starting pool of nucleic acids, the SELEX™ method includes steps of: (a) contacting the mixture with the target under conditions favorable for binding; (b) partitioning unbound nucleic acids from those nucleic acids which have bound specifically to target molecules; (c) dissociating the nucleic acid-target complexes; (d) amplifying the nucleic acids dissociated from the nucleic acid-target complexes to yield a ligand-enriched mixture of nucleic acids; and (e) reiterating the steps of binding, partitioning, dissociating and amplifying through as many cycles as desired to yield highly specific, high affinity nucleic acid ligands to the target molecule. In those instances where RNA aptamers are being selected, the SELEX™ method further comprises the steps of: (i) reverse transcribing the nucleic acids dissociated from the nucleic acid-target complexes before amplification in step (d); and (ii) transcribing the amplified nucleic acids from step (d) before restarting the process.

Within a nucleic acid mixture containing a large number of possible sequences and structures, there is a wide range of binding affinities for a given target. A nucleic acid mixture comprising, for example, a 20 nucleotide randomized segment can have 4²⁰ candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands or aptamers.

Cycles of selection and amplification are repeated until a desired goal is achieved. In the most general case, selection/amplification is continued until no significant improvement in binding strength is achieved on repetition of the cycle. The method is typically used to sample approximately 10¹⁴ different nucleic acid species but may be used to sample as many as about 10¹⁸ different nucleic acid species. Generally, nucleic acid aptamer molecules are selected in a 5 to 20 cycle procedure. In one embodiment, heterogeneity is introduced only in the initial selection stages and does not occur throughout the replicating process.

In one embodiment of SELEX™, the selection process is so efficient at isolating those nucleic acid ligands that bind most strongly to the selected target, that only one cycle of selection and amplification is required. Such an efficient selection may occur, for example, in a chromatographic-type process wherein the ability of nucleic acids to associate with targets bound on a column operates in such a manner that the column is sufficiently able to allow separation and isolation of the highest affinity nucleic acid ligands.

In many cases, it is not necessarily desirable to perform the iterative steps of SELEX™ until a single nucleic acid ligand is identified. The target-specific nucleic acid ligand solution may include a family of nucleic acid structures or motifs that have a number of conserved sequences and a number of sequences which can be substituted or added without significantly affecting the affinity of the nucleic acid ligands to the target. By terminating the SELEX™ process prior to completion, it is possible to determine the sequence of a number of members of the nucleic acid ligand solution family.

A variety of nucleic acid primary, secondary and tertiary structures are known to exist. The structures or motifs that have been shown most commonly to be involved in non-Watson-Crick type interactions are referred to as hairpin loops, symmetric and asymmetric bulges, pseudoknots and myriad combinations of the same. Almost all known cases of such motifs suggest that they can be formed in a nucleic acid sequence of no more than 30 nucleotides. For this reason, it is often preferred that SELEX™ procedures with contiguous randomized segments be initiated with nucleic acid sequences containing a randomized segment of between about 20 to about 50 nucleotides and in some embodiments, about 30 to about 40 nucleotides. In one example, the 5′-fixed:random:3′-fixed sequence comprises a random sequence of about 30 to about 50 nucleotides.

The core SELEX™ method has been modified to achieve a number of specific objectives. For example, U.S. Pat. No. 5,707,796 describes the use of SELEX™ in conjunction with gel electrophoresis to select nucleic acid molecules with specific structural characteristics, such as bent DNA. U.S. Pat. No. 5,763,177 describes SELEX™ based methods for selecting nucleic acid ligands containing photo reactive groups capable of binding and/or photo-crosslinking to and/or photo-inactivating a target molecule. U.S. Pat. No. 5,567,588 and U.S. Pat. No. 5,861,254 describe SELEX™ based methods which achieve highly efficient partitioning between oligonucleotides having high and low affinity for a target molecule. U.S. Pat. No. 5,496,938 describes methods for obtaining improved nucleic acid ligands after the SELEX™ process has been performed. U.S. Pat. No. 5,705,337 describes methods for covalently linking a ligand to its target.

SELEX™ can also be used to obtain nucleic acid ligands that bind to more than one site on the target molecule, and to obtain nucleic acid ligands that include non-nucleic acid species that bind to specific sites on the target. SELEX™ provides means for isolating and identifying nucleic acid ligands which bind to any envisionable target, including large and small biomolecules such as nucleic acid-binding proteins and proteins not known to bind nucleic acids as part of their biological function as well as cofactors and other small molecules. For example, U.S. Pat. No. 5,580,737 discloses nucleic acid sequences identified through SELEX™ which are capable of binding with high affinity to caffeine and the closely related analog, theophylline.

Counter-SELEX™ is a method for improving the specificity of nucleic acid ligands to a target molecule by eliminating nucleic acid ligand sequences with cross-reactivity to one or more non-target molecules. Counter-SELEX™ is comprised of the steps of: (a) preparing a candidate mixture of nucleic acids; (b) contacting the candidate mixture with the target, wherein nucleic acids having an increased affinity to the target relative to the candidate mixture may be partitioned from the remainder of the candidate mixture; (c) partitioning the increased affinity nucleic acids from the remainder of the candidate mixture; (d) dissociating the increased affinity nucleic acids from the target; (e) contacting the increased affinity nucleic acids with one or more non-target molecules such that nucleic acid ligands with specific affinity for the non-target molecule(s) are removed; and (f) amplifying the nucleic acids with specific affinity only to the target molecule to yield a mixture of nucleic acids enriched for nucleic acid sequences with a relatively higher affinity and specificity for binding to the target molecule. As described above for SELEX™, cycles of selection and amplification are repeated as necessary until a desired goal is achieved.

One potential problem encountered in the use of nucleic acids as therapeutics and vaccines is that oligonucleotides in their phosphodiester form may be quickly degraded in body fluids by intracellular and extracellular enzymes such as endonucleases and exonucleases before the desired effect is manifest. The SELEX™ method thus encompasses the identification of high-affinity nucleic acid ligands containing modified nucleotides conferring improved characteristics on the ligand, such as improved in vivo stability or improved delivery characteristics. Examples of such modifications include chemical substitutions at the ribose and/or phosphate and/or base positions. SELEX™-identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-O-methyl (2′-OMe) substituents.

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Modifications to generate oligonucleotide populations which are resistant to nucleases can also include one or more substitute internucleotide linkages, altered sugars, altered bases, or combinations thereof. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, and unusual base-pairing combinations such as the isobases isocytidine and isoguanosine. Modifications can also include 3′ and 5′ modifications such as capping.

In one embodiment, oligonucleotides are provided in which the P(O)O group is replaced by P(O)S (“thioate”), P(S)S (“dithioate”), P(O)NR₂ (“amidate”), P(O)R, P(O)OR′, CO or CH₂ (“formacetal”) or 3′-amine (—NH—CH₂—CH₂—), wherein each R or R′ is independently H or substituted or unsubstituted alkyl. Linkage groups can be attached to adjacent nucleotides through an —O—, —N—, or —S— linkage. Not all linkages in the oligonucleotide are required to be identical. As used herein, the term phosphorothioate encompasses one or more non-bridging oxygen atoms in a phosphodiester bond replaced by one or more sulfur atom.

In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al., Nucl. Acid Res. 19:2629-2635 (1991); and Hobbs, et al., Biochemistry 12:5138-5145 (1973). Other modifications are known to one of ordinary skill in the art. Such modifications may be pre-SELEX™ process modifications or post-SELEX™ process modifications (modification of previously identified unmodified ligands) or may be made by incorporation into the SELEX™ process.

Pre-SELEX™ process modifications or those made by incorporation into the SELEX™ process yield nucleic acid ligands with both specificity for their SELEX™ target and improved stability, e.g., in vivo stability. Post-SELEX™ process modifications made to nucleic acid ligands may result in improved stability, e.g., in vivo stability without adversely affecting the binding capacity of the nucleic acid ligand.

The SELEX™ method encompasses combining selected oligonucleotides with other selected oligonucleotides and non-oligonucleotide functional units as described in U.S. Pat. No. 5,637,459 and U.S. Pat. No. 5,683,867. The SELEX™ method further encompasses combining selected nucleic acid ligands with lipophilic or non-immunogenic high molecular weight compounds in a diagnostic or therapeutic complex, as described, e.g., in U.S. Pat. No. 6,011,020, U.S. Pat. No. 6,051,698, and PCT Publication No. WO 98/18480. These patents and applications teach the combination of a broad array of shapes and other properties, with the efficient amplification and replication properties of oligonucleotides, and with the desirable properties of other molecules.

The identification of nucleic acid ligands to small, flexible peptides via the SELEX™ method has also been explored. Small peptides have flexible structures and usually exist in solution in an equilibrium of multiple conformers, and thus it was initially thought that binding affinities may be limited by the conformational entropy lost upon binding a flexible peptide. However, the feasibility of identifying nucleic acid ligands to small peptides in solution was demonstrated in U.S. Pat. No. 5,648,214. In this patent, high affinity RNA nucleic acid ligands to substance P, an 11 amino acid peptide, were identified.

The aptamers with specificity and binding affinity to the target(s) of the present invention are typically selected by the SELEX™ process as described herein. As part of the SELEX™ process, the sequences selected to bind to the target are then optionally minimized to determine the minimal sequence having the desired binding affinity. The selected sequences and/or the minimized sequences are optionally optimized by performing random or directed mutagenesis of the sequence to increase binding affinity or alternatively to determine which positions in the sequence are essential for binding activity. Additionally, selections can be performed with sequences incorporating modified nucleotides to stabilize the aptamer molecules against degradation in vivo.

### 2′ Modified SELEX™

In order for an aptamer to be suitable for use as a therapeutic, it is preferably inexpensive to synthesize, safe and stable in vivo. Wild-type RNA and DNA aptamers are typically not stable in vivo because of their susceptibility to degradation by nucleases. Resistance to nuclease degradation can be greatly increased by the incorporation of modifying groups at the 2′-position.

Fluoro and amino groups have been successfully incorporated into oligonucleotide pools from which aptamers have been subsequently selected. However, these modifications greatly increase the cost of synthesis of the resultant aptamer, and may introduce safety concerns in some cases because of the possibility that the modified nucleotides could be recycled into host DNA by degradation of the modified oligonucleotides and subsequent use of the nucleotides as substrates for DNA synthesis.

Aptamers that contain 2′-O-methyl (“2′-OMe”) nucleotides, as provided herein, overcome many of these drawbacks. Oligonucleotides containing 2′-OMe nucleotides are nuclease-resistant and inexpensive to synthesize. Although 2′-OMe nucleotides are ubiquitous in biological systems, natural polymerases do not accept 2′-OMe NTPs as substrates under physiological conditions, thus there are no safety concerns over the recycling of 2′-OMe nucleotides into host DNA. The SELEX™ method used to generate 2′-modified aptamers is described, e.g., in U.S. Provisional Patent Application Ser. No. 60/430,761, filed Dec. 3, 2002, U.S. Provisional Patent Application Ser. No. 60/487,474, filed Jul. 15, 2003, U.S. Provisional Patent Application Ser. No. 60/517,039, filed Nov. 4, 2003, U.S. patent application Ser. No. 10/729,581, filed Dec. 3, 2003, and U.S. patent application Ser. No. 10/873,856, filed Jun. 21, 2004, entitled “Method for in vitro Selection of 2′-O-methyl Substituted Nucleic Acids”, each of which is herein incorporated by reference in its entirety.

The present invention includes aptamers that bind to and modulate the function of IL-23 and/or IL-12 which contain modified nucleotides (e.g., nucleotides which have a modification at the 2′ position) to make the oligonucleotide more stable than the unmodified oligonucleotide to enzymatic and chemical degradation as well as thermal and physical degradation. Although there are several examples of 2′-OMe containing aptamers in the literature (see, e.g., Green et al., Current Biology 2, 683-695, 1995) these were generated by the in vitro selection of libraries of modified transcripts in which the C and U residues were 2′-fluoro (2′-F) substituted and the A and G residues were 2′-OH. Once functional sequences were identified then each A and G residue was tested for tolerance to 2′-OMe substitution, and the aptamer was re-synthesized having all A and G residues which tolerated 2′-OMe substitution as 2′-OMe residues. Most of the A and G residues of aptamers generated in this two-step fashion tolerate substitution with 2′-OMe residues, although, on average, approximately 20% do not. Consequently, aptamers generated using this method tend to contain from two to four 2′-OH residues, and stability and cost of synthesis are compromised as a result. By incorporating modified nucleotides into the transcription reaction which generate stabilized oligonucleotides used in oligonucleotide pools from which aptamers are selected and enriched by SELEX™ (and/or any of its variations and improvements, including those described herein), the methods of the present invention eliminate the need for stabilizing the selected aptamer oligonucleotides (e.g., by resynthesizing the aptamer oligonucleotides with modified nucleotides).

In one embodiment, the present invention provides aptamers comprising combinations of 2′-OH, 2′-F, 2′-deoxy, and 2′-OMe modifications of the ATP, GTP, CTP, TTP, and UTP nucleotides. In another embodiment, the present invention provides aptamers comprising combinations of 2′-OH, 2′-F, 2′-deoxy, 2′-OMe, 2′-NH₂, and 2′-methoxyethyl modifications of the ATP, GTP, CTP, TTP, and UTP nucleotides. In another embodiment, the present invention provides aptamers comprising 56 combinations of 2′-OH, 2′-F, 2′-deoxy, 2′-OMe, 2′-NH₂, and 2′-methoxyethyl modifications of the ATP, GTP, CTP, TTP, and UTP nucleotides.

2′ modified aptamers of the invention are created using modified polymerases, e.g., a modified T7 polymerase, having a rate of incorporation of modified nucleotides having bulky substituents at the furanose 2′ position that is higher than that of wild-type polymerases. For example, a single mutant T7 polymerase (Y639F) in which the tyrosine residue at position 639 has been changed to phenylalanine readily utilizes 2′deoxy, 2′amino-, and 2′fluoro-nucleotide triphosphates (NTPs) as substrates and has been widely used to synthesize modified RNAs for a variety of applications. However, this mutant T7 polymerase reportedly can not readily utilize (i.e., incorporate) NTPs with bulky 2′-substituents such as 2′-OMe or 2′-azido (2′-N₃) substituents. For incorporation of bulky 2′ substituents, a double T7 polymerase mutant (Y639F/H784A) having the histidine at position 784 changed to an alanine residue in addition to the Y639F mutation has been described and has been used in limited circumstances to incorporate modified pyrimidine NTPs. See Padilla, R. and Sousa, R., Nucleic Acids Res., 2002, 30(24): 138. A single mutant T7 polymerase (H784A) having the histidine at position 784 changed to an alanine residue has also been described. Padilla et al., Nucleic Acids Research, 2002, 30: 138. In both the Y639F/H784A double mutant and H784A single mutant T7 polymerases, the change to a smaller amino acid residue such as alanine allows for the incorporation of bulkier nucleotide substrates, e.g., 2′-OMe substituted nucleotides.

Generally, it has been found that under the conditions disclosed herein, the Y693F single mutant can be used for the incorporation of all 2′-OMe substituted NTPs except GTP and the Y639F/H784A double mutant can be used for the incorporation of all 2′-OMe substituted NTPs including GTP. It is expected that the H784A single mutant possesses properties similar to the Y639F and the Y639F/H784A mutants when used under the conditions disclosed herein.

2′-modified oligonucleotides may be synthesized entirely of modified nucleotides, or with a subset of modified nucleotides. The modifications can be the same or different. All nucleotides may be modified, and all may contain the same modification. All nucleotides may be modified, but contain different modifications, e.g., all nucleotides containing the same base may have one type of modification, while nucleotides containing other bases may have different types of modification. All purine nucleotides may have one type of modification (or are unmodified), while all pyrimidine nucleotides have another, different type of modification (or are unmodified). In this way, transcripts, or pools of transcripts are generated using any combination of modifications, including for example, ribonucleotides (2′-OH), deoxyribonucleotides (2′-deoxy), 2′-F, and 2′-OMe nucleotides. A transcription mixture containing 2′-OMe C and U and 2′-OH A and G is referred to as an “rRmY” mixture and aptamers selected therefrom are referred to as “rRmY” aptamers. A transcription mixture containing deoxy A and G and 2′-OMe U and C is referred to as a “dRmY” mixture and aptamers selected therefrom are referred to as “dRmY” aptamers. A transcription mixture containing 2′-OMe A, C, and U, and 2′-OH G is referred to as a “rGmH” mixture and aptamers selected therefrom are referred to as “rGmH” aptamers. A transcription mixture alternately containing 2′-OMe A, C, U and G and 2′-OMe A, U and C and 2′-F G is referred to as an “alternating mixture” and aptamers selected therefrom are referred to as “alternating mixture” aptamers. A transcription mixture containing 2′-OMe A, U, C, and G, where up to 10% of the G's are ribonucleotides is referred to as a “r/mGmH” mixture and aptamers selected therefrom are referred to as “r/mGmH” aptamers. A transcription mixture containing 2′-OMe A, U, and C, and 2′-F G is referred to as a “fGmH” mixture and aptamers selected therefrom are referred to as “fGmH” aptamers. A transcription mixture containing 2′-OMe A, U, and C, and deoxy G is referred to as a “dGmH” mixture and aptamers selected therefrom are referred to as “dGmH” aptamers. A transcription mixture containing deoxy A, and 2′-OMe C, G and U is referred to as a “dAmB” mixture and aptamers selected therefrom are referred to as “dAmB” aptamers, and a transcription mixture containing all 2′-OH nucleotides is referred to as a “rN” mixture and aptamers selected therefrom are referred to as “rN” or “rRrY” aptamers. A “mRmY” aptamer is one containing all 2′-O-methyl nucleotides and is usually derived from a r/mGmH oligonucleotide by post-SELEX™ replacement, when possible, of any 2′-OH Gs with 2′-OMe Gs.

A preferred embodiment includes any combination of 2′-OH, 2′-deoxy and 2′-OMe nucleotides. A more preferred embodiment includes any combination of 2′-deoxy and 2′-OMe nucleotides. An even more preferred embodiment is with any combination of 2′-deoxy and 2′-OMe nucleotides in which the pyrimidines are 2′-OMe (such as dRmY, mRmY or dGmH).

Incorporation of modified nucleotides into the aptamers of the invention is accomplished before (pre-) the selection process (e.g., a pre-SELEX™ process modification). Optionally, aptamers of the invention in which modified nucleotides have been incorporated by pre-SELEX™ process modification can be further modified by post-SELEX™ process modification (i.e., a post-SELEX™ process modification after a pre-SELEX™ modification). Pre-SELEX™ process modifications yield modified nucleic acid ligands with specificity for the SELEX™ target and also improved in vivo stability. Post-SELEX™ process modifications, i.e., modification (e.g., truncation, deletion, substitution or additional nucleotide modifications of previously identified ligands having nucleotides incorporated by pre-SELEX™ process modification) can result in a further improvement of in vivo stability without adversely affecting the binding capacity of the nucleic acid ligand having nucleotides incorporated by pre-SELEX™ process modification.

To generate pools of 2′-modified (e.g., 2′-OMe) RNA transcripts in conditions under which a polymerase accepts 2′-modified NTPs the preferred polymerase is the Y693F/H784A double mutant or the Y693F single mutant. Other polymerases, particularly those that exhibit a high tolerance for bulky 2′-substituents, may also be used in the present invention. Such polymerases can be screened for this capability by assaying their ability to incorporate modified nucleotides under the transcription conditions disclosed herein.

A number of factors have been determined to be important for the transcription conditions useful in the methods disclosed herein. For example, increases in the yields of modified transcript are observed when a leader sequence is incorporated into the 5′ end of a fixed sequence at the 5′ end of the DNA transcription template, such that at least about the first 6 residues of the resultant transcript are all purines.

Another important factor in obtaining transcripts incorporating modified nucleotides is the presence or concentration of 2′-OH GTP. Transcription can be divided into two phases: the first phase is initiation, during which an NTP is added to the 3′-hydroxyl end of GTP (or another substituted guanosine) to yield a dinucleotide which is then extended by about 10-12 nucleotides; the second phase is elongation, during which transcription proceeds beyond the addition of the first about 10-12 nucleotides. It has been found that small amounts of 2′-OH GTP added to a transcription mixture containing an excess of 2′-OMe GTP are sufficient to enable the polymerase to initiate transcription using 2′-OH GTP, but once transcription enters the elongation phase the reduced discrimination between 2′-OMe and 2′-OH GTP, and the excess of 2′-OMe GTP over 2′-OH GTP allows the incorporation of principally the 2′-OMe GTP.

Another important factor in the incorporation of 2′-OMe substituted nucleotides into transcripts is the use of both divalent magnesium and manganese in the transcription mixture. Different combinations of concentrations of magnesium chloride and manganese chloride have been found to affect yields of 2′-O-methylated transcripts, the optimum concentration of the magnesium and manganese chloride being dependent on the concentration in the transcription reaction mixture of NTPs which complex divalent metal ions. To obtain the greatest yields of maximally 2′ substituted O-methylated transcripts (i.e., all A, C, and U and about 90% of G nucleotides), concentrations of approximately 5 mM magnesium chloride and 1.5 mM manganese chloride are preferred when each NTP is present at a concentration of 0.5 mM. When the concentration of each NTP is 1.0 mM, concentrations of approximately 6.5 mM magnesium chloride and 2.0 mM manganese chloride are preferred. When the concentration of each NTP is 2.0 mM, concentrations of approximately 9.6 mM magnesium chloride and 2.9 mM manganese chloride are preferred. In any case, departures from these concentrations of up to two-fold still give significant amounts of modified transcripts.

Priming transcription with GMP or guanosine is also important. This effect results from the specificity of the polymerase for the initiating nucleotide. As a result, the 5′-terminal nucleotide of any transcript generated in this fashion is likely to be 2′-OH G. The preferred concentration of GMP (or guanosine) is 0.5 mM and even more preferably 1 mM. It has also been found that including PEG, preferably PEG-8000, in the transcription reaction is useful to maximize incorporation of modified nucleotides.

For maximum incorporation of 2′-OMe ATP (100%), UTP (100%), CTP (100%) and GTP (˜90%) (“r/mGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (6.5 mM where the concentration of each 2′-OMe NTP is 1.0 mM), MnCl₂ 1.5 mM (2.0 mM where the concentration of each 2′-OMe NTP is 1.0 mM), 2′-OMe NTP (each) 500 μM (more preferably, 1.0 mM), 2′-OH GTP 30 μM, 2′-OH GMP 500 μM, pH 7.5, Y639F/H784A T7 RNA Polymerase 15 units/mL, inorganic pyrophosphatase 5 units/mL, and an all-purine leader sequence of at least 8 nucleotides long. As used herein, one unit of the Y639F/H784A mutant T7 RNA polymerase (or any other mutant T7 RNA polymerase specified herein) is defined as the amount of enzyme required to incorporate 1 nmole of 2′-OMe NTPs into transcripts under the r/mGmH conditions. As used herein, one unit of inorganic pyrophosphatase is defined as the amount of enzyme that will liberate 1.0 mole of inorganic orthophosphate per minute at pH 7.2 and 25° C.

For maximum incorporation (100%) of 2′-OMe ATP, UTP and CTP (“rGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F T7 RNA Polymerase 15 units/mL, inorganic pyrophosphatase 5 units/mL, and an all-purine leader sequence of at least 8 nucleotides long.

For maximum incorporation (100%) of 2′-OMe UTP and CTP (“rRmY”) into transcripts the following conditions are preferred: HEPES buffer 200 mM, DTT 40 mM, spermidine 2 mM, PEG-8000 10% (w/v), Triton X-100 0.01% (w/v), MgCl₂ 5 mM (9.6 mM where the concentration of each 2′-OMe NTP is 2.0 mM), MnCl₂ 1.5 mM (2.9 mM where the concentration of each 2′-OMe NTP is 2.0 mM), 2′-OMe NTP (each) 500 μM (more preferably, 2.0 mM), pH 7.5, Y639F/H784A T7 RNA Polymerase 15 units/mL, inorganic pyrophosphatase 5 units/mL, and an all-purine leader sequence of at least 8 nucleotides long.

For maximum incorporation (100%) of deoxy ATP and GTP and 2′-OMe UTP and CTP (“dRmY”) into transcripts the following conditions are preferred: HEPES buffer 200 mM, DTT 40 mM, spermine 2 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/mL, inorganic pyrophosphatase 5 units/mL, and an all-purine leader sequence of at least 8 nucleotides long.

For maximum incorporation (100%) of 2′-OMe ATP, UTP and CTP and 2′-F GTP (“fGmH”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/mL, inorganic pyrophosphatase 5 units/mL, and an all-purine leader sequence of at least 8 nucleotides long.

For maximum incorporation (100%) of deoxy ATP and 2′-OMe UTP, GTP and CTP (“dAmB”) into transcripts the following conditions are preferred: HEPES buffer 200 mM, DTT 40 mM, spermidine 2 mM, PEG-8000 10% (w/v), Triton X-100 0.01% (w/v), MgCl₂ 9.6 mM, MnCl₂ 2.9 mM, 2′-OMe NTP (each) 2.0 mM, pH 7.5, Y639F T7 RNA Polymerase 15 units/mL, inorganic pyrophosphatase 5 units/mL, and an all-purine leader sequence of at least 8 nucleotides long.

For each of the above (a) transcription is preferably performed at a temperature of from about 20° C. to about 50° C., preferably from about 30° C. to 45° C., and more preferably at about 37° C. for a period of at least two hours and (b) 50-300 nM of a double stranded DNA transcription template is used (200 nM template is used in round 1 to increase diversity (300 nM template is used in dRmY transcriptions)), and for subsequent rounds approximately 50 nM, a 1/10 dilution of an optimized PCR reaction, using conditions described herein, is used). The preferred DNA transcription templates are described below (where ARC254 and ARC256 transcribe under all 2′-OMe conditions and ARC255 transcribes under rRmY conditions).

SEQ ID NO 1 (ARC254)
5′-CATCGATGCTAGTCGTAACGATCCNNNNNNNNNNNNNNNNNNNNNNN
NNNNNNNCGAGAACGTTCTCTCCTCTCCCTATAGTGAGTCGTATTA-3′
SEQ ID NO 2 (ARC255)
5′-CATGCATCGCGACTGACTAGCCGNNNNNNNNNNNNNNNNNNNNNNNN
NNNNNNGTAGAACGTTCTCCTCTCCCTATAGTGAGTCGTATTA-3′
SEQ ID NO 3 (ARC256)
5′-CATCGATCGATCGATCGACAGCGNNNNNNNNNNNNNNNNNNNNNNNN
NNNNNNGTAGAACGTTCTCTCCTCTCCCTATAGTGAGTCGTATTA-3′

Under rN transcription conditions of the present invention, the transcription reaction mixture comprises 2′-OH adenosine triphosphates (ATP), 2′-OH guanosine triphosphates (GTP), 2′-OH cytidine triphosphates (CTP), and 2′-OH uridine triphosphates (UTP). The modified oligonucleotides produced using the rN transcription mixtures of the present invention comprise substantially all 2′-OH adenosine, 2′-OH guanosine, 2′-OH cytidine, and 2′-OH uridine. In a preferred embodiment of rN transcription, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-OH cytidine, and at least 80% of all uridine nucleotides are 2′-OH uridine. In a more preferred embodiment of rN transcription, the resulting modified oligonucleotides of the present invention comprise a sequence where at least 90% of all adenosine nucleotides are 2′-OH adenosine, at least 90% of all guanosine nucleotides are 2′-OH guanosine, at least 90% of all cytidine nucleotides are 2′-OH cytidine, and at least 90% of all uridine nucleotides are 2′-OH uridine. In a most preferred embodiment of rN transcription, the modified oligonucleotides of the present invention comprise a sequence where 100% of all adenosine nucleotides are 2′-OH adenosine, 100% of all guanosine nucleotides are 2′-OH guanosine, 100% of all cytidine nucleotides are 2′-OH cytidine, and 100% of all uridine nucleotides are 2′-OH uridine.

Under rRmY transcription conditions of the present invention, the transcription reaction mixture comprises 2′-OH adenosine triphosphates, 2′-OH guanosine triphosphates, 2′-O-methyl cytidine triphosphates, and 2′-O-methyl uridine triphosphates. The modified oligonucleotides produced using the rRmY transcription mixtures of the present invention comprise substantially all 2′-OH adenosine, 2′-OH guanosine, 2′-O-methyl cytidine and 2′-O-methyl uridine. In a preferred embodiment, the resulting modified oligonucleotides comprise a sequence where at least 80% of all adenosine nucleotides are 2′-OH adenosine, at least 80% of all guanosine nucleotides are 2′-OH guanosine, at least 80% of all cytidine nucleotides are 2′-O-methyl cytidine and at least 80% of all uridine nucleotides are 2′-O-methyl uridine. In a more preferred embodiment, the resulting modified oligonucleotides comprise a sequence where at least 90% of all adenosine nucleotides are 2′-OH adenosine, at least 90% of all guanosine nucleotides are 2′-OH guanosine, at least 90% of all cytidine nucleotides are 2′-O-methyl cytidine and at least 90% of all uridine nucleotides are 2′-O-methyl uridine In a most preferred embodiment, the resulting modified oligonucleotides comprise a sequence where 100% of all adenosine nucleotides are 2′-OH adenosine, 100% of all guanosine nucleotides are 2′-OH guanosine, 100% of all cytidine nucleotides are 2′-O-methyl cytidine and 100% of all uridine nucleotides are 2′-O-methyl uridine.

Under dRmY transcription conditions of the present invention, the transcription reaction mixture comprises 2′-deoxy adenosine triphosphates, 2′-deoxy guanosine triphosphates, 2′-O-methyl cytidine triphosphates, and 2′-O-methyl uridine triphosphates. The modified oligonucleotides produced using the dRmY transcription conditions of the present invention comprise substantially all 2′-deoxy adenosine, 2′-deoxy guanosine, 2′-O-methyl cytidine, and 2′-O-methyl uridine. In a preferred embodiment, the resulting modified oligonucleotides of the present invention comprise a sequence where at least 80% of all adenosine nucleotides are 2′-deoxy adenosine, at least 80% of all guanosine nucleotides are 2′-deoxy guanosine, at least 80% of all cytidine nucleotides are 2′-O-methyl cytidine, and at least 80% of all uridine nucleotides are 2′-O-methyl uridine. In a more preferred embodiment, the resulting modified oligonucleotides of the present invention comprise a sequence where at least 90% of all adenosine nucleotides are 2′-deoxy adenosine, at least 90% of all guanosine nucleotides are 2′-deoxy guanosine, at least 90% of all cytidine nucleotides are 2′-O-methyl cytidine, and at least 90% of all uridine nucleotides are 2′-O-methyl uridine. In a most preferred embodiment, the resulting modified oligonucleotides of the present invention comprise a sequence where 100% of all adenosine nucleotides are 2′-deoxy adenosine, 100% of all guanosine nucleotides are 2′-deoxy guanosine, 100% of all cytidine nucleotides are 2′-O-methyl cytidine, and 100% of all uridine nucleotides are 2′-O-methyl uridine.

Under rGmH transcription conditions of the present invention, the transcription reaction mixture comprises 2′-OH guanosine triphosphates, 2′-O-methyl cytidine triphosphates, 2′-O-methyl uridine triphosphates, and 2′-O-methyl adenosine triphosphates. The modified oligonucleotides produced using the rGmH transcription mixtures of the present invention comprise substantially all 2′-OH guanosine, 2′-O-methyl cytidine, 2′-O-methyl uridine, and 2′-O-methyl adenosine. In a preferred embodiment, the resulting modified oligonucleotides comprise a sequence where at least 80% of all guanosine nucleotides are 2′-OH guanosine, at least 80% of all cytidine nucleotides are 2′-O-methyl cytidine, at least 80% of all uridine nucleotides are 2′-O-methyl uridine, and at least 80% of all adenosine nucleotides are 2′-O-methyl adenosine. In a more preferred embodiment, the resulting modified oligonucleotides comprise a sequence where at least 90% of all guanosine nucleotides are 2′-OH guanosine, at least 90% of all cytidine nucleotides are 2′-O-methyl cytidine, at least 90% of all uridine nucleotides are 2′-O-methyl uridine, and at least 90% of all adenosine nucleotides are 2′-O-methyl adenosine. In a most preferred embodiment, the resulting modified oligonucleotides comprise a sequence where 100% of all guanosine nucleotides are 2′-OH guanosine, 100% of all cytidine nucleotides are 2′-O-methyl cytidine, 100% of all uridine nucleotides are 2′-O-methyl uridine, and 100% of all adenosine nucleotides are 2′-O-methyl adenosine.

Under r/mGmH transcription conditions of the present invention, the transcription reaction mixture comprises 2′-O-methyl adenosine triphosphate, 2′-O-methyl cytidine triphosphate, 2′-O-methyl guanosine triphosphate, 2′-O-methyl uridine triphosphate and 2′-OH guanosine triphosphate. The resulting modified oligonucleotides produced using the r/mGmH transcription mixtures of the present invention comprise substantially all 2′-O-methyl adenosine, 2′-O-methyl cytidine, 2′-O-methyl guanosine, and 2′-O-methyl uridine, wherein the population of guanosine nucleotides has a maximum of about 10% 2′-OH guanosine. In a preferred embodiment, the resulting r/mGmH modified oligonucleotides of the present invention comprise a sequence where at least 80% of all adenosine nucleotides are 2′-O-methyl adenosine, at least 80% of all cytidine nucleotides are 2′-O-methyl cytidine, at least 80% of all guanosine nucleotides are 2′-O-methyl guanosine, at least 80% of all uridine nucleotides are 2′-O-methyl uridine, and no more than about 10% of all guanosine nucleotides are 2′-OH guanosine. In a more preferred embodiment, the resulting modified oligonucleotides comprise a sequence where at least 90% of all adenosine nucleotides are 2′-O-methyl adenosine, at least 90% of all cytidine nucleotides are 2′-O-methyl cytidine, at least 90% of all guanosine nucleotides are 2′-O-methyl guanosine, at least 90% of all uridine nucleotides are 2′-O-methyl uridine, and no more than about 10% of all guanosine nucleotides are 2′-OH guanosine. In a most preferred embodiment, the resulting modified oligonucleotides comprise a sequence where 100% of all adenosine nucleotides are 2′-O-methyl adenosine, 100% of all cytidine nucleotides are 2′-O-methyl cytidine, 90% of all guanosine nucleotides are 2′-O-methyl guanosine, and 100% of all uridine nucleotides are 2′-O-methyl uridine, and no more than about 10% of all guanosine nucleotides are 2′-OH guanosine.

Under fGmH transcription conditions of the present invention, the transcription reaction mixture comprises 2′-O-methyl adenosine triphosphates, 2′-O-methyl uridine triphosphates, 2′-O-methyl cytidine triphosphates, and 2′-F guanosine triphosphates. The modified oligonucleotides produced using the fGmH transcription conditions of the present invention comprise substantially all 2′-O-methyl adenosine, 2′-O-methyl uridine, 2′-O-methyl cytidine, and 2′-F guanosine. In a preferred embodiment, the resulting modified oligonucleotides comprise a sequence where at least 80% of all adenosine nucleotides are 2′-O-methyl adenosine, at least 80% of all uridine nucleotides are 2′-O-methyl uridine, at least 80% of all cytidine nucleotides are 2′-O-methyl cytidine, and at least 80% of all guanosine nucleotides are 2′-F guanosine. In a more preferred embodiment, the resulting modified oligonucleotides comprise a sequence where at least 90% of all adenosine nucleotides are 2′-O-methyl adenosine, at least 90% of all uridine nucleotides are 2′-O-methyl uridine, at least 90% of all cytidine nucleotides are 2′-O-methyl cytidine, and at least 90% of all guanosine nucleotides are 2′-F guanosine. In a most preferred embodiment, the resulting modified oligonucleotides comprise a sequence where 100% of all adenosine nucleotides are 2′-O-methyl adenosine, 100% of all uridine nucleotides are 2′-O-methyl uridine, 100% of all cytidine nucleotides are 2′-O-methyl cytidine, and 100% of all guanosine nucleotides are 2′-F guanosine.

Under dAmB transcription conditions of the present invention, the transcription reaction mixture comprises 2′-deoxy adenosine triphosphates, 2′-O-methyl cytidine triphosphates, 2′-O-methyl guanosine triphosphates, and 2′-O-methyl uridine triphosphates. The modified oligonucleotides produced using the dAmB transcription mixtures of the present invention comprise substantially all 2′-deoxy adenosine, 2′-O-methyl cytidine, 2′-O-methyl guanosine, and 2′-O-methyl uridine. In a preferred embodiment, the resulting modified oligonucleotides comprise a sequence where at least 80% of all adenosine nucleotides are 2′-deoxy adenosine, at least 80% of all cytidine nucleotides are 2′-O-methyl cytidine, at least 80% of all guanosine nucleotides are 2′-O-methyl guanosine, and at least 80% of all uridine nucleotides are 2′-O-methyl uridine. In a more preferred embodiment, the resulting modified oligonucleotides comprise a sequence where at least 90% of all adenosine nucleotides are 2′-deoxy adenosine, at least 90% of all cytidine nucleotides are 2′-O-methyl cytidine, at least 90% of all guanosine nucleotides are 2′-O-methyl guanosine, and at least 90% of all uridine nucleotides are 2′-O-methyl uridine. In a most preferred embodiment, the resulting modified oligonucleotides of the present invention comprise a sequence where 100% of all adenosine nucleotides are 2′-deoxy adenosine, 100% of all cytidine nucleotides are 2′-O-methyl cytidine, 100% of all guanosine nucleotides are 2′-O-methyl guanosine, and 100% of all uridine nucleotides are 2′-O-methyl uridine.

In each case, the transcription products can then be used as the library in the SELEX™ process to identify aptamers and/or to determine a conserved motif of sequences that have binding specificity to a given target. The resulting sequences are already partially stabilized, eliminating this step from the process to arrive at an optimized aptamer sequence and giving a more highly stabilized aptamer as a result. Another advantage of the 2′-OMe SELEX™ process is that the resulting sequences are likely to have fewer 2′-OH nucleotides required in the sequence, possibly none. To the extent 2′OH nucleotides remain they can be removed by performing post-SELEX™ modifications.

As described below, lower but still useful yields of transcripts fully incorporating 2′ substituted nucleotides can be obtained under conditions other than the optimized conditions described above. For example, variations to the above transcription conditions include:

The HEPES buffer concentration can range from 0 to 1 M. The present invention also contemplates the use of other buffering agents having a pKa between 5 and 10 including, for example, Tris-hydroxymethyl-aminomethane.

The DTT concentration can range from 0 to 400 mM. The methods of the present invention also provide for the use of other reducing agents including, for example, mercaptoethanol.

The spermidine and/or spermine concentration can range from 0 to 20 mM.

The PEG-8000 concentration can range from 0 to 50% (w/v). The methods of the present invention also provide for the use of other hydrophilic polymer including, for example, other molecular weight PEG or other polyalkylene glycols.

The Triton X-100 concentration can range from 0 to 0.1% (w/v). The methods of the present invention also provide for the use of other non-ionic detergents including, for example, other detergents, including other Triton-X detergents.

The MgCl₂ concentration can range from 0.5 mM to 50 mM. The MnCl₂ concentration can range from 0.15 mM to 15 mM. Both MgCl₂ and MnCl₂ must be present within the ranges described and in a preferred embodiment are present in about a 10 to about 3 ratio of MgCl₂:MnCl₂, preferably, the ratio is about 3-5:1, more preferably, the ratio is about 3-4:1.

The 2′-OMe NTP concentration (each NTP) can range from 5 μM to 5 mM.

The 2′-OH GTP concentration can range from 0 μM to 300 μM.

The 2′-OH GMP concentration can range from 0 to 5 mM.

The pH can range from pH 6 to pH 9. The methods of the present invention can be practiced within the pH range of activity of most polymerases that incorporate modified nucleotides. In addition, the methods of the present invention provide for the optional use of chelating agents in the transcription reaction condition including, for example, EDTA, EGTA, and DTT.

### IL-23 and/or IL-12 Aptamer Selection Strategies

The present invention provides aptamers that bind to human IL-23 and/or IL-12 and in some embodiments, inhibit binding to their receptor and/or otherwise modulate their function. Human IL-23 and IL-12 are both heterodimers that have one subunit in common and one unique. The subunit in common is the p40 subunit which contains the following amino acid sequence (Accession # AF180563) (SEQ ID NO 4):

MCHQQLVISWFSLVFLASPLVAIWELKKDVYVVELDWYPDAPGE
MVVLTCDTPEEDGITWTLDQSSEVLGSGKTLTIQVKEFGDAGQYTCHKGG
EVLSHSLLLLHKKEDGIWSTDILKDQKEPKNKTFLRCEAKNYSGRFTCWW
LTTISTDLTFSVKSSRGSSDPQGVTCGAATLSAERVRGDNKEYEYSVECQ
EDSACPAAEESLPIEVMVDAVHKLKYENYTSSFFIRDIIKPDPPKNLQLK
PLKNSRQVEVSWEYPDTWSTPHSYFSLTFCVQVQGKSKREKKDRVFTDKT
SATVICRKNASISVRAQDRYYSSSWSEWASVPCS.

The p19 subunit is unique to IL-23 and contains the following amino acid sequence (Accession # BC067511) (SEQ ID NO 5):

MLGSRAVMLLLLLPWTAQGRAVPGGSSPAWTQCQQLSQKLCTLA
WSAHPLVGHMDLREEGDEETTNDVPHIQCGDGCDPQGLRDNSQFCLQRIH
QGLIFYEKLLGSDIFTGEPSLLPDSPVGQLHASLLGLSQLLQPEGHHWET
QQIPSLSPSQPWQRLLLRFKILRSLQAFVAVAARVFAHGAATLSP.

The p35 subunit is unique to IL-12 and contains the following amino acid sequence (Accession # AF180562) (SEQ ID NO 6):

MWPPGSASQPPPSPAAATGLHPAARPVSLQCRLSMCPARSLLLV
ATLVLLDHLSLARNLPVATPDPGMFPCLHHSQNLLRAVSNMLQKARQTLE
FYPCTSEEIDHEDITKDKTSTVEACLPLELTKNESCLNSRETSFITNGSC
LASRKTSFMMALCLSSIYEDLKMYQVEFKTMNAKLLMDPKRQIFLDQNML
AVIDELMQALNFNSETVPQKSSLEEPDFYKTKIKLCILLHAFRIRAVTID
RVMSYLNAS.

The present invention also provides aptamers that bind to mouse IL-23 and/or IL-12 and in some embodiments, inhibit binding to their receptor and/or otherwise modulate their function. Like human, mouse IL-23 and IL-12 are both heterodimers that share the mouse p40 subunit, while the mouse p19 subunit is specific to mouse IL-23 and the mouse p35 subunit is unique to mouse IL-12. The mouse p40 subunit contains the following amino acid sequence (Accession # P43432) (SEQ ID NO 315):

MCPQKLTISWFAIVLLVSPLMAMWELEKDVYVVEVDWTPDAPGETVNLTC
DTPEEDDITWTSDQRHGVIGSGKTLTITVKEFLDAGQYTCHKGGETLSHS
HLLLHKKENGIWSTEILKNFKNKTFLKCEAPNYSGRFTCSWLVQRNMDLK
FNIKSSSSSPDSRAVTCGMASLSAEKVTLDQRDYEKYSVSCQEDVTCPTA
EETLPIELALEARQQNKYENYSTSFFIRDIIKPDPPKNLQMKPLKNSQVE
VSWEYPDSWSTPHSYFSLKFFVRIQRKKEKMKETEEGCNQKGAFLVEKTS
TEVQCKGGNVCVQAQDRYYNSSCSKWACVPCRVRS

The mouse p19 subunit contains the following amino acid sequence (Accession # NP112542) (SEQ ID NO 316):

MLDCRAVIMLWLLPWVTQGLAVPRSSSPDWAQCQQLSRNLCMLAWNAHAP
AGHMNLLREEEDEETKNNVPRIQCEDGCDPQGLKDNSQFCLQRIRQGLAF
YKHLLDSDIFKGEPALLPDSPMEQLHTSLLGLSQLLQPEDHPRETQQMPS
LSSSQQWQRPLLRSKILRSLQAFLAIAARVFAHGAATLTE PLVPTA

The mouse p35 subunit contains the following amino acid sequence (Accession # P43431) (SEQ ID NO 317):

MCQSRYLLFLATLALLNHLSLARVIPVSGPARCLSQSRNLLKTTDDMVKT
AREKLKHYSCTAEDIDHEDITRDQTSTLKTCLPLELHKNESCLATRETSS
TTRGSCLPPQKTSLMMTLCLGSIYEDLKMYQTEFQAINAALQNHNHQQII
LDKGMLVAIDELMQSLNHNGETLRQKPPVGEADPYRVKMKLCILLHAFST
RVVTINRVMG YLSSA

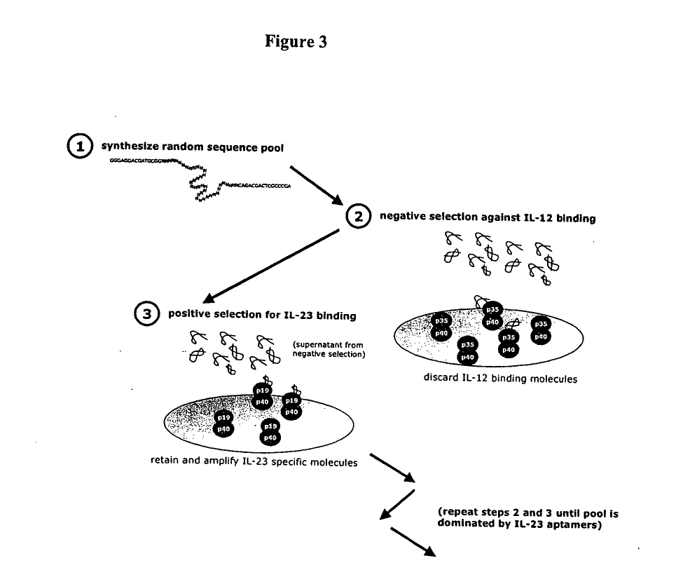

Several SELEX™ strategies can be employed to generate aptamers with a variety of specificities for IL-23 and IL-12. One scheme produces aptamers specific for IL-23 over IL-12 by including IL-12 in a negative selection step. This eliminates sequences that recognize the common subunit, p40 (SEQ ID NO 4), and selects for aptamers specific to IL-23, or the p19 subunit (SEQ ID NO 5) as shown in FIG. 3. One scheme produces aptamers specific for IL-12 over IL-23 by including IL-23 in the negative selection step. This eliminates sequences that recognize the common subunit, p40 (SEQ ID NO 4) and selects for aptamers specific for IL-12, or the p35 subunit (SEQ ID NO 6). A separate selection in which IL-23 and IL-12 are alternated every other round elicits aptamers that recognize the common subunit, p40 (SEQ ID NO 4), and therefore recognizes both proteins. Once sequences with the desired binding specificity are found, minimization of those sequences can be undertaken to systematically reduce the size of the sequences with concomitant improvement in binding characteristics.

The selected aptamers having the highest affinity and specific binding as demonstrated by biological assays as described in the examples below are suitable therapeutics for treating conditions in which IL-23 and/or IL-12 is involved in pathogenesis.

### IL-23/IL-12 Specific Binding Aptamers

The materials of the present invention comprise a series of nucleic acid aptamers of ˜25-90 nucleotides in length which bind specifically to cytokines of the human IL-12 cytokine family which includes IL-12, IL-23, and IL-27; p19, p35, and p40 subunit monomers; and p40 subunit dimers; and which functionally modulate, e.g., block, the activity of IL-23 and/or IL-12 in in vivo and/or in cell-based assays.

Aptamers specifically capable of binding and modulating IL-23 and/or IL-12 are set forth herein. These aptamers provide a low-toxicity, safe, and effective modality of treating and/or preventing autoimmune and inflammatory related diseases or disorders. In one embodiment, the aptamers of the invention are used to treat and/or prevent inflammatory and autoimmune diseases, including but not limited to, multiple sclerosis, rheumatoid arthritis, psoriasis vulgaris, and irritable bowel disease, including without limitation Crohn's disease, and ulcerative colitis, each of which are known to be caused by or otherwise associated with the IL-23 and/or IL-12 cytokine. In another embodiment, the aptamers of the invention are used to treat and/or prevent Type I Diabetes, which is known to be caused by or otherwise associated with the IL-23 and/or IL-12 cytokine. In another embodiment, the aptamers of the invention are used to treat and/or prevent other indications for which activation of cytokine receptor binding is desirable including, for example, systemic lupus erythamatosus, colon cancer, lung cancer, and bone resorption in osteoporosis.

Examples of IL-23 and/or IL-12 specific binding aptamers for use as therapeutics and/or diagnostics include the following sequences listed below.

Unless noted otherwise, ARC489 (SEQ ID NO 91), ARC491 (SEQ ID NO 94), ARC621 (SEQ ID NO 108), ARC627 (SEQ ID NO 110), ARC527 (SEQ ID NO 159), ARC792 (SEQ ID NO 162), ARC794 (SEQ ID NO 164), ARC795 (SEQ ID NO 165), ARC979 (SEQ ID NO 177), ARC1386 (SEQ ID NO 224), and ARC1623-ARC1625 (SEQ ID NOs 309-311) represent the sequences of the aptamers that bind to IL-23 and/or IL-12 that were selected under SELEX™ conditions in which the purines (A and G) are deoxy, and the pyrimidines (C and U) are 2′-OMe.

The unique sequence region of ARC489 (SEQ ID NO 91) and ARC491 (SEQ ID NO 94) begins at nucleotide 23, immediately following the sequence GGGAGAGGAGAGAACGUUCUAC (SEQ ID NO 69), and runs until it meets the 3′fixed nucleic acid sequence GCUGUCGAUCGAUCGAUCGAUG (SEQ ID NO 90).

The unique sequence region of ARC621 (SEQ ID NO 108) and ARC627 (SEQ ID NO 110) begins at nucleotide 23, immediately following the sequence GGGAGAGGAGAGAACGUUCUAC (SEQ ID NO 101), and runs until it meets the 3′fixed nucleic acid sequence GUCGAUCGAUCGAUCAUCGAUG (SEQ ID NO 102).

SEQ ID NO 91 (ARC489)
GGGAGAGGAGAGAACGUUCUACAGCGCCGGUGGGCGGGCAUUGGGUGGAU
GCGCUGUCGAUCGAUCGAUCGAUG
SEQ ID NO 94 (ARC491)
GGGAGAGGAGAGAACGUUCUACAGCGCCGGUGGGUGGGCAUAGGGUGGAU
GCGCUGUCGAUCGAUCGAUCGAUG
SEQ ID NO 108 (ARC621)
GGGAGAGGAGAGAACGUUCUACAGGCGGUUACGGGGGAUGCGGGUGGGAC
AGGUCGAUCGAUCGAUCAUCGAUG
SEQ ID NO 110 (ARC627)
GGGAGAGGAGAGAACGUUCUACAGGCAAGUAAUUGGGGAGUGCGGGCGGG
GUGUCGAUCGAUCGAUCAUCGAUG
SEQ ID NO 159 (ARC527)
ACAGCGCCGGUGGGCGGGCAUUGGGUGGAUGCGCUGU
SEQ ID NO 162 (ARC792)
GGCAAGUAAUUGGGGAGUGCGGGCGGGG
SEQ ID NO 164 (ARC794)
GGCGGUACGGGGAGUGUGGGUUGGGGCCGG
SEQ ID NO 165 (ARC795)
CGAUAUAGGCGGUACGGGGGGAGUGGGCUGGGGUCG
SEQ ID NO 177 (ARC979)
ACAGGCAAGUAAUUGGGGAGUGCGGGCGGGGUGU

ARC1623 (SEQ ID NO 309), ARC1624 (SEQ ID NO 310) and ARC1625 (SEQ ID NO 311) represent optimized sequences based on ARC979 (SEQ ID NO 177), where “d” stands for deoxy, “m” stands for 2′-O-methyl, “s” indicates a phosphorothioate internucleotide linkage, and “3T” stands for a 3′-inverted deoxy thymidine.

SEQ ID NO 309 (ARC1623)
dAmCdAdGdGmCdAdAdGmUdAdAmUmUdGmGmG-s-dG-s-dA-s-dGm
U-s-dGmCmGmGdGmCdGdGmGmGmUdGmU-3T
SEQ ID NO 310 (ARC1624)
dAmCdAdGdGmCdAdAdGmUdAdAmUmUdGmGmGdGdAdGmUdGmCmGm
G-s-dGmC-s-dG-s-dGmGmGmUdGmU-3T
SEQ ID NO 311 (ARC1625)
dAmCdAdGdGmCdAdAdGmUdAdAmUmUdGmGmGdGdAdGmUdGmCmGm
GdGmCdGdGmGmGmU-s-dGmU-3T

SEQ ID NOS 139-140, SEQ ID NOS 144-145, SEQ ID NO 147, and SEQ ID NOS 151-152, represent the sequences of the aptamers that bind to IL-23 and/or IL-12 that were selected under SELEX™ conditions in which the purines (A and G) are 2′-OH (ribo) and the pyrimidines (C and U) are 2′-Fluoro.

SEQ ID NO 139 (A10.min5)
GGAGCAUACACAAGAAGUUUUUUGUGCUCUGAGUACUCAGCGUCCGUAAG
GGAUAUGCUCC
SEQ ID NO 140 (A10.min6)
GGAGUACGCCGAAAGGCGCUCUGAGUACUCAGCGUCCGUAAGGGAUACU
CC
SEQ ID NO 144 (B10.min4)
GGAGCAUACACAAGAAGUGCUUCAUGCGGCAAACUGCAUGACGUCGAAUA
GAUAUGCUCC
SEQ ID NO 145 (B10.min5)
GGAGUACACAAGAAGUGCUUCCGAAAGGACGUCGAAUAGAUACUCC
SEQ ID NO 147 (F11.min2)
GGACAUACACAAGAUGUGCUUGAGUUAAAUCUCAUCGUCCCCGUUUGGGG
AUAUGUC
SEQ ID NO 151
GGGUACGCCGAAAGGCGCUUCCGAAAGGACGUCCGUAAGGGAUACCC
SEQ ID NO 152
GGAGUACGCCGAAAGGCGCUUCCGAAAGGACGUCCGUAAGGGAAUACUCC

Other aptamers that bind IL-23 and/or IL-12 are described below in Examples 1-3.

These aptamers may include modifications as described herein including e.g., conjugation to lipophilic or high molecular weight compounds (e.g., PEG), incorporation of a CpG motif, incorporation of a capping moiety, incorporation of modified nucleotides, and incorporation of phosphorothioate in the phosphate backbone.

In one embodiment, an isolated, non-naturally occurring aptamer that binds to IL-23 and/or IL-12 is provided. In some embodiments, the isolated, non-naturally occurring aptamer has a dissociation constant (“K_{D}”) for IL-23 and/or IL-12 of less than 100 μM, less than 1 μM, less than 500 nM, less than 100 nM, less than 50 nM, less than 1 nM, less than 500 μM, less than 100 μM, and less than 50 μM. In some embodiments of the invention, the dissociation constant is determined by dot blot titration as described in Example 1 below.

In another embodiment, the aptamer of the invention modulates a function of IL-23 and/or IL-12. In another embodiment, the aptamer of the invention inhibits an IL-23 and/or IL-12 function while in another embodiment the aptamer stimulates a function of the target. In another embodiment of the invention, the aptamer binds and/or modulates a function of an IL-23 or IL-12 variant. An IL-23 or IL-12 variant as used herein encompasses variants that perform essentially the same function as an IL-23 or IL-12 function, preferably comprises substantially the same structure and in some embodiments comprises at least 70% sequence identity, preferably at least 80% sequence identity, more preferably at least 90% sequence identity, and more preferably at least 95% sequence identity to the amino acid sequence of IL-23 or IL-12. In some embodiments of the invention, the sequence identity of target variants is determined using BLAST as described below.

The terms “sequence identity” in the context of two or more nucleic acid or protein sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity is the algorithm used in the basic local alignment search tool (hereinafter “BLAST”), see, e.g. Altschul et al., J. Mol. Biol. 215: 403-410 (1990) and Altschul et al., Nucleic Acids Res., 15: 3389-3402 (1997). Software for performing BLAST analyses is publicly available through the National Center for Biotechnology Information (hereinafter “NCBI”). The default parameters used in determining sequence identity using the software available from NCBI, e.g., BLASTN (for nucleotide sequences) and BLASTP (for amino acid sequences) are described in McGinnis et al., Nucleic Acids Res., 32: W20-W25 (2004).

In one embodiment of the invention, the aptamer has substantially the same ability to bind to IL-23 as that of an aptamer comprising any one of SEQ ID NOs 13-66, SEQ ID NOs 71-88, SEQ ID NOs 91-96, SEQ ID NOs 103-118, SEQ ID NOs 124-134, SEQ ID NOs 135-159, SEQ ID NO 162, and SEQ ID NOs 164-172, SEQ ID NOs 176-178, SEQ ID NOs 181-196, and SEQ ID NOs 199-314. In another embodiment of the invention, the aptamer has substantially the same structure and ability to bind to IL-23 as that of an aptamer comprising any one of SEQ ID NOs 13-66, SEQ ID NOs 71-88, SEQ ID NOs 91-96, SEQ ID NOs 103-118, SEQ ID NOs 124-134, SEQ ID NOs 135-159, SEQ ID NO 162, and SEQ ID NOs 164-172, SEQ ID NOs 176-178, SEQ ID NOs 181-196, and SEQ ID NOs 199-314.

In one embodiment of the invention, the aptamer has substantially the same ability to bind to IL-23 and/or IL-12 as that of an aptamer comprising any one of SEQ ID NO 14, SEQ ID NOs 17-19, SEQ ID NO 21, SEQ ID NOs 27-32, SEQ ID NOs 34-40, SEQ ID NO 42, SEQ ID NO 49, SEQ ID NOs 60-61, SEQ ID NOs 91-92, SEQ ID NO 94, and SEQ ID NOs 103-118. In another embodiment of the invention, the aptamer has substantially the same structure and ability to bind to IL-23 and/or IL-12 as that of an aptamer comprising any one of SEQ ID NO 14, SEQ ID NOs 17-19, SEQ ID NO 21, SEQ ID NOs 27-32, SEQ ID NOs 34-40, SEQ ID NO 42, SEQ ID NO 49, SEQ ID NOs 60-61, SEQ ID NOs 91-92, SEQ ID NO 94, and SEQ ID NOs 103-118.

In another embodiment, the aptamers of the invention are used as an active ingredient in pharmaceutical compositions. In another embodiment, the aptamers or compositions comprising the aptamers of the invention are used to treat inflammatory and autoimmune diseases (including but not limited to, multiple sclerosis, rheumatoid arthritis, psoriasis vulgaris, systemic lupus erythamatosus, and irritable bowel disease, including without limitation Crohn's disease, and ulcerative colitis), Type I Diabetes, colon cancer, lung cancer, and bone resorption in osteoporosis.

In some embodiments aptamer therapeutics of the present invention have great affinity and specificity to their targets while reducing the deleterious side effects from non-naturally occurring nucleotide substitutions if the aptamer therapeutics break down in the body of patients or subjects. In some embodiments, the therapeutic compositions containing the aptamer therapeutics of the present invention are free of or have a reduced amount of fluorinated nucleotides.

The aptamers of the present invention can be synthesized using any oligonucleotide synthesis techniques known in the art including solid phase oligonucleotide synthesis techniques (see, e.g., Froehler et al., Nucl. Acid Res. 14:5399-5467 (1986) and Froehler et al., Tet. Lett. 27:5575-5578 (1986)) and solution phase methods well known in the art such as triester synthesis methods (see, e.g., Sood et al., Nucl. Acid Res. 4:2557 (1977) and Hirose et al., Tet. Lett., 28:2449 (1978)).

### Aptamers Having Immunostimulatory Motifs

The present invention provides aptamers that bind to IL-23 and/or IL-12 and modulate their biological function. More specifically, the present invention provides aptamers that increase the binding of IL-23 and/or IL-12 to the IL-23 and/or IL-12 receptor thereby enhancing the biological function of IL-23 and/or IL-12. The agonistic effect of such aptamers can be further enhanced by selecting for aptamers which bind to the IL-23 and/or IL-12 and contain immunostimulatory motifs, or by treating with aptamers which bind to IL-23 and/or IL-12 in conjunction with aptamers to a target known to bind immunostimulatory sequences.

Recognition of bacterial DNA by the vertebrate immune system is based on the recognition of unmethylated CG dinucleotides in particular sequence contexts (“CpG motifs”). One receptor that recognizes such a motif is Toll-like receptor 9 (“TLR 9”), a member of a family of Toll-like receptors (˜10 members) that participate in the innate immune response by recognizing distinct microbial components. TLR 9 binds unmethylated oligodeoxynucleotide (“ODN”) CpG sequences in a sequence-specific manner. The recognition of CpG motifs triggers defense mechanisms leading to innate and ultimately acquired immune responses. For example, activation of TLR 9 in mice induces activation of antigen presenting cells, up regulation of MHC class I and II molecules and expression of important co-stimulatory molecules and cytokines including IL-12 and IL-23. This activation both directly and indirectly enhances B and T cell responses, including robust up regulation of the TH1 cytokine IFN-gamma. Collectively, the response to CpG sequences leads to: protection against infectious diseases, improved immune response to vaccines, an effective response against asthma, and improved antibody-dependent cell-mediated cytotoxicity. Thus, CpG ODNs can provide protection against infectious diseases, function as immuno-adjuvants or cancer therapeutics (monotherapy or in combination with a mAb or other therapies), and can decrease asthma and allergic response.

Aptamers of the present invention comprising one or more CpG or other immunostimulatory sequences can be identified or generated by a variety of strategies using, e.g., the SELEX™ process described herein. The incorporated immunostimulatory sequences can be DNA, RNA and/or a combination DNA/RNA. In general the strategies can be divided into two groups. In group one, the strategies are directed to identifying or generating aptamers comprising both a CpG motif or other immunostimulatory sequence as well as a binding site for a target, where the target (hereinafter “non-CpG target”) is a target other than one known to recognize CpG motifs or other immunostimulatory sequences and known to stimulates an immune response upon binding to a CpG motif. In some embodiments of the invention the non-CpG target is an IL-23 and/or IL12 target. The first strategy of this group comprises performing SELEX™ to obtain an aptamer to a specific non-CpG target, preferably a target, e.g., IL-23 and/or IL-12, where a repressed immune response is relevant to disease development, using an oligonucleotide pool wherein a CpG motif has been incorporated into each member of the pool as, or as part of, a fixed region, e.g., in some embodiments the randomized region of the pool members comprises a fixed region having a CpG motif incorporated therein, and identifying an aptamer comprising a CpG motif. The second strategy of this group comprises performing SELEX™ to obtain an aptamer to a specific non-CpG target preferably a target, e.g., IL-23 and/or IL-12, where a repressed immune response is relevant to disease development, and following selection appending a CpG motif to the 5′ and/or 3′ end or engineering a CpG motif into a region, preferably a non-essential region, of the aptamer. The third strategy of this group comprises performing SELEX™ to obtain an aptamer to a specific non-CpG target, preferably a target, e.g., IL-23 and/or IL-12, where a repressed immune response is relevant to disease development, wherein during synthesis of the pool the molar ratio of the various nucleotides is biased in one or more nucleotide addition steps so that the randomized region of each member of the pool is enriched in CpG motifs, and identifying an aptamer comprising a CpG motif. The fourth strategy of this group comprises performing SELEX™ to obtain an aptamer to a specific non-CpG target, preferably a target, e.g., IL-23 and/or IL-112, where a repressed immune response is relevant to disease development, and identifying an aptamer comprising a CpG motif. The fifth strategy of this group comprises performing SELEX™ to obtain an aptamer to a specific non-CpG target, preferably a target, e.g., IL-23 and/or IL-12, where a repressed immune response is relevant to disease development, and identifying an aptamer which, upon binding, stimulates an immune response but which does not comprise a CpG motif.

In group two, the strategies are directed to identifying or generating aptamers comprising a CpG motif and/or other sequences that are bound by the receptors for the CpG motifs (e.g., TLR9 or the other toll-like receptors) and upon binding stimulate an immune response. The first strategy of this group comprises performing SELEX™ to obtain an aptamer to a target known to bind to CpG motifs or other immunostimulatory sequences and upon binding stimulate an immune response using an oligonucleotide pool wherein a CpG motif has been incorporated into each member of the pool as, or as part of, a fixed region, e.g., in some embodiments the randomized region of the pool members comprise a fixed region having a CpG motif incorporated therein, and identifying an aptamer comprising a CpG motif. The second strategy of this group comprises performing SELEX™ to obtain an aptamer to a target known to bind to CpG motifs or other immunostimulatory sequences and upon binding stimulate an immune response and then appending a CpG motif to the 5′ and/or 3′ end or engineering a CpG motif into a region, preferably a non-essential region, of the aptamer. The third strategy of this group comprises performing SELEX™ to obtain an aptamer to a target known to bind to CpG motifs or other immunostimulatory sequences and upon binding stimulate an immune response wherein during synthesis of the pool, the molar ratio of the various nucleotides is biased in one or more nucleotide addition steps so that the randomized region of each member of the pool is enriched in CpG motifs, and identifying an aptamer comprising a CpG motif. The fourth strategy of this group comprises performing SELEX™ to obtain an aptamer to a target known to bind to CpG motifs or other immunostimulatory sequences and upon binding stimulate an immune response and identifying an aptamer comprising a CpG motif. The fifth strategy of this group comprises performing SELEX™ to obtain an aptamer to a target known to bind to CpG motifs or other immunostimulatory sequences, and identifying an aptamer which upon binding, stimulate an immune response but which does not comprise a CpG motif.

A variety of different classes of CpG motifs have been identified, each resulting upon recognition in a different cascade of events, release of cytokines and other molecules, and activation of certain cell types. See, e.g., CpG Motifs in Bacterial DNA and Their Immune Effects, Annu. Rev. Immunol. 2002, 20:709-760, incorporated herein by reference. Additional immunostimulatory motifs are disclosed in the following U.S. patents, each of which is incorporated herein by reference: U.S. Pat. No. 6,207,646; U.S. Pat. No. 6,239,116; U.S. Pat. No. 6,429,199; U.S. Pat. No. 6,214,806; U.S. Pat. No. 6,653,292; U.S. Pat. No. 6,426,434; U.S. Pat. No. 6,514,948 and U.S. Pat. No. 6,498,148. Any of these CpG or other immunostimulatory motifs can be incorporated into an aptamer. The choice of aptamers is dependent on the disease or disorder to be treated. Preferred immunostimulatory motifs are as follows (shown 5′ to 3′ left to right) wherein “r” designates a purine, “y” designates a pyrimidine, and “X” designates any nucleotide: AACGTTCGAG (SEQ ID NO 7); AACGTT; ACGT, rCGy; rrCGyy, XCGX, XXCGXX, and X₁X₂CGY₁Y₂ wherein X₁ is G or A, X₂ is not C, Y₁ is not G and Y₂ is preferably T.

In those instances where a CpG motif is incorporated into an aptamer that binds to a specific target other than a target known to bind to CpG motifs and upon binding stimulate an immune response (a “non-CpG target”), the CpG is preferably located in a non-essential region of the aptamer. Non-essential regions of aptamers can be identified by site-directed mutagenesis, deletion analyses and/or substitution analyses. However, any location that does not significantly interfere with the ability of the aptamer to bind to the non-CpG target may be used. In addition to being embedded within the aptamer sequence, the CpG motif may be appended to either or both of the 5′ and 3′ ends or otherwise attached to the aptamer. Any location or means of attachment may be used so long as the ability of the aptamer to bind to the non-CpG target is not significantly interfered with.

As used herein, “stimulation of an immune response” can mean either (1) the induction of a specific response (e.g., induction of a Th1 response) or of the production of certain molecules or (2) the inhibition or suppression of a specific response (e.g., inhibition or suppression of the Th2 response) or of certain molecules.

### Pharmaceutical Compositions

The invention also includes pharmaceutical compositions containing aptamer molecules that bind to IL-23 and/or IL-12. In some embodiments, the compositions are suitable for internal use and include an effective amount of a pharmacologically active compound of the invention, alone or in combination, with one or more pharmaceutically acceptable carriers. The compounds are especially useful in that they have very low, if any toxicity.

Compositions of the invention can be used to treat or prevent a pathology, such as a disease or disorder, or alleviate the symptoms of such disease or disorder in a patient. For example, compositions of the present invention can be used to treat or prevent a pathology associated with IL-23 and/or IL-12 cytokines, including inflammatory and autoimmune related diseases, Type I Diabetes, bone resorption in osteoporosis, and cancer.

Compositions of the invention are useful for administration to a subject suffering from, or predisposed to, a disease or disorder which is related to or derived from a target to which the aptamers of the invention specifically bind. Compositions of the invention can be used in a method for treating a patient or subject having a pathology. The method involves administering to the patient or subject an aptamer or a composition comprising aptamers that bind to IL-23 and/or IL-12 involved with the pathology, so that binding of the aptamer to the IL-23 and/or IL-12 alters the biological function of the target, thereby treating the pathology.

The patient or subject having a pathology, i.e., the patient or subject treated by the methods of this invention, can be a vertebrate, more particularly a mammal, or more particularly a human.

In practice, the aptamers or their pharmaceutically acceptable salts, are administered in amounts which will be sufficient to exert their desired biological activity, e.g., inhibiting the binding of the IL-23 and/or IL-12 to its receptor.

One aspect of the invention comprises an aptamer composition of the invention in combination with other treatments for inflammatory and autoimmune diseases, cancer, and other related disorders. The aptamer composition of the invention may contain, for example, more than one aptamer. In some examples, an aptamer composition of the invention, containing one or more compounds of the invention, is administered in combination with another useful composition such as an anti-inflammatory agent, an immunosuppressant, an antiviral agent, or the like. Furthermore, the compounds of the invention may be administered in combination with a cytotoxic, cytostatic, or chemotherapeutic agent such as an alkylating agent, anti-metabolite, mitotic inhibitor or cytotoxic antibiotic, as described above. In general, the currently available dosage forms of the known therapeutic agents for use in such combinations will be suitable.

“Combination therapy” (or “co-therapy”) includes the administration of an aptamer composition of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

“Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents.

Sequential or substantially simultaneous administration of each therapeutic agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutic agent of the combination selected may be administered by injection while the other therapeutic agents of the combination may be administered topically.

Alternatively, for example, all therapeutic agents may be administered topically or all therapeutic agents may be administered by injection. The sequence in which the therapeutic agents are administered is not narrowly critical unless noted otherwise. “Combination therapy” also can embrace the administration of the therapeutic agents as described above in further combination with other biologically active ingredients. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Therapeutic or pharmacological compositions of the present invention will generally comprise an effective amount of the active component(s) of the therapy, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the therapeutic compositions of the present invention.

The preparation of pharmaceutical or pharmacological compositions will be known to those of skill in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or sponge.

Upon formulation, therapeutics will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the host animal to be treated. Precise amounts of active compound required for administration depend on the judgment of the practitioner and are peculiar to each individual.

A minimal volume of a composition required to disperse the active compounds is typically utilized. Suitable regimes for administration are also variable, but would be typified by initially administering the compound and monitoring the results and then giving further controlled doses at further intervals.

For instance, for oral administration in the form of a tablet or capsule (e.g., a gelatin capsule), the active drug component can be combined with an oral, non-toxic, pharmaceutically acceptable inert carrier such as ethanol, glycerol, water and the like. Moreover, when desired or necessary, suitable binders, lubricants, disintegrating agents, and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes, and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol, and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

The compounds of the invention can also be administered in such oral dosage forms as timed release and sustained release tablets or capsules, pills, powders, granules, elixirs, tinctures, suspensions, syrups and emulsions. Suppositories are advantageously prepared from fatty emulsions or suspensions.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parenteral injectable administration is generally used for subcutaneous, intramuscular or intravenous injections and infusions. Additionally, one approach for parenteral administration employs the implantation of a slow-release or sustained-released systems, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, inhalants, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would typically range from 0.01% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like. The active compound defined above, may be also formulated as suppositories, using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564. For example, the aptamer molecules described herein can be provided as a complex with a lipophilic compound or non-immunogenic, high molecular weight compound constructed using methods known in the art. An example of nucleic-acid associated complexes is provided in U.S. Pat. No. 6,011,020.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, and triethanolamine oleate.

The dosage regimen utilizing the aptamers is selected in accordance with a variety of factors including type, species, age, weight, sex and medical condition of the patient; the severity of the condition to be treated; the route of administration; the renal and hepatic function of the patient; and the particular aptamer or salt thereof employed. An ordinarily skilled physician or veterinarian can readily determine and prescribe the effective amount of the drug required to prevent, counter or arrest the progress of the condition.

Oral dosages of the present invention, when used for the indicated effects, will range between about 0.05 to 7500 mg/day orally. The compositions are preferably provided in the form of scored tablets containing 0.5, 1.0, 2.5, 5.0, 10.0, 15.0, 25.0, 50.0, 100.0, 250.0, 500.0 and 1000.0 mg of active ingredient. Infused dosages, intranasal dosages and transdermal dosages will range between 0.05 to 7500 mg/day. Subcutaneous, intravenous and intraperitoneal dosages will range between 0.05 to 3800 mg/day.

Effective plasma levels of the compounds of the present invention range from 0.002 mg/mL to 50 mg/mL.

Compounds of the present invention may be administered in a single daily dose, or the total daily dosage may be administered in divided doses of two, three or four times daily.

### Modulation of Pharmacokinetics and Biodistribution of Aptamer Therapeutics

It is important that the pharmacokinetic properties for all oligonucleotide-based therapeutics, including aptamers, be tailored to match the desired pharmaceutical application. While aptamers directed against extracellular targets do not suffer from difficulties associated with intracellular delivery (as is the case with antisense and RNAi-based therapeutics), such aptamers must still be able to be distributed to target organs and tissues, and remain in the body (unmodified) for a period of time consistent with the desired dosing regimen.

Thus, the present invention provides materials and methods to affect the pharmacokinetics of aptamer compositions, and, in particular, the ability to tune aptamer pharmacokinetics. The tunability of (i.e., the ability to modulate) aptamer pharmacokinetics is achieved through conjugation of modifying moieties (e.g., PEG polymers) to the aptamer and/or the incorporation of modified nucleotides (e.g., 2′-fluoro or 2′-O-methyl) to alter the chemical composition of the nucleic acid. The ability to tune aptamer pharmacokinetics is used in the improvement of existing therapeutic applications, or alternatively, in the development of new therapeutic applications. For example, in some therapeutic applications, e.g., in anti-neoplastic or acute care settings where rapid drug clearance or turn-off may be desired, it is desirable to decrease the residence times of aptamers in the circulation. Alternatively, in other therapeutic applications, e.g., maintenance therapies where systemic circulation of a therapeutic is desired, it may be desirable to increase the residence times of aptamers in circulation.

In addition, the tunability of aptamer pharmacokinetics is used to modify the biodistribution of an aptamer therapeutic in a subject. For example, in some therapeutic applications, it may be desirable to alter the biodistribution of an aptamer therapeutic in an effort to target a particular type of tissue or a specific organ (or set of organs). In these applications, the aptamer therapeutic preferentially accumulates in a specific tissue or organ(s). In other therapeutic applications, it may be desirable to target tissues displaying a cellular marker or a symptom associated with a given disease, cellular injury or other abnormal pathology, such that the aptamer therapeutic preferentially accumulates in the affected tissue. For example, as described in copending provisional application U.S. Ser. No. 60/550,790, filed on Mar. 5, 2004, and entitled “Controlled Modulation of the Pharmacokinetics and Biodistribution of Aptamer Therapeutics”, and in the non-provisional application U.S. Ser. No. 10/______, filed on Mar. 7, 2005, also entitled “Controlled Modulation of the Pharmacokinetics and Biodistribution of Aptamer Therapeutics”, PEGylation of an aptamer therapeutic (e.g., PEGylation with a 20 kDa PEG polymer) is used to target inflamed tissues, such that the PEGylated aptamer therapeutic preferentially accumulates in inflamed tissue.

To determine the pharmacokinetic and biodistribution profiles of aptamer therapeutics (e.g., aptamer conjugates or aptamers having altered chemistries, such as modified nucleotides) a variety of parameters are monitored. Such parameters include, for example, the half-life (t_{1/2}), the plasma clearance (C1), the volume of distribution (Vss), the area under the concentration-time curve (AUC), maximum observed serum or plasma concentration (Cₘₐₓ), and the mean residence time (MRT) of an aptamer composition. As used herein, the term “AUC” refers to the area under the plot of the plasma concentration of an aptamer therapeutic versus the time after aptamer administration. The AUC value is used to estimate the bioavailability (i.e., the percentage of administered aptamer therapeutic in the circulation after aptamer administration) and/or total clearance (C1) (i.e., the rate at which the aptamer therapeutic is removed from circulation) of a given aptamer therapeutic. The volume of distribution relates the plasma concentration of an aptamer therapeutic to the amount of aptamer present in the body. The larger the Vss, the more an aptamer is found outside of the plasma (i.e., the more extravasation).

The present invention provides materials and methods to modulate, in a controlled manner, the pharmacokinetics and biodistribution of stabilized aptamer compositions in vivo by conjugating an aptamer to a modulating moiety such as a small molecule, peptide, or polymer terminal group, or by incorporating modified nucleotides into an aptamer. As described herein, conjugation of a modifying moiety and/or altering nucleotide(s) chemical composition alters fundamental aspects of aptamer residence time in circulation and distribution to tissues.

In addition to clearance by nucleases, oligonucleotide therapeutics are subject to elimination via renal filtration. As such, a nuclease-resistant oligonucleotide administered intravenously typically exhibits an in vivo half-life of <10 min, unless filtration can be blocked. This can be accomplished by either facilitating rapid distribution out of the blood stream into tissues or by increasing the apparent molecular weight of the oligonucleotide above the effective size cut-off for the glomerulus. Conjugation of small therapeutics to a PEG polymer (PEGylation), described below, can dramatically lengthen residence times of aptamers in circulation, thereby decreasing dosing frequency and enhancing effectiveness against vascular targets.

Aptamers can be conjugated to a variety of modifying moieties, such as high molecular weight polymers, e.g., PEG; peptides, e.g., Tat (a 13-amino acid fragment of the HIV Tat protein (Vives, et al., (1997), J. Biol. Chem. 272(25): 16010-7)), Ant (a 16-amino acid sequence derived from the third helix of the Drosophila antennapedia homeotic protein (Pietersz, et al., (2001), Vaccine 19(11-12): 1397-405)) and Arg7 (a short, positively charged cell-permeating peptides composed of polyarginine (Arg₇) (Rothbard, et al., (2000), Nat. Med. 6(11): 1253-7; Rothbard, J et al., (2002), J. Med. Chem. 45(17): 3612-8)); and small molecules, e.g., lipophilic compounds such as cholesterol. Among the various conjugates described herein, in vivo properties of aptamers are altered most profoundly by complexation with PEG groups. For example, complexation of a mixed 2° F. and 2′-OMe modified aptamer therapeutic with a 20 kDa PEG polymer hinders renal filtration and promotes aptamer distribution to both healthy and inflamed tissues. Furthermore, the 20 kDa PEG polymer-aptamer conjugate proves nearly as effective as a 40 kDa PEG polymer in preventing renal filtration of aptamers. While one effect of PEGylation is on aptamer clearance, the prolonged systemic exposure afforded by presence of the 20 kDa moiety also facilitates distribution of aptamer to tissues, particularly those of highly perfused organs and those at the site of inflammation. The aptamer-20 kDa PEG polymer conjugate directs aptamer distribution to the site of inflammation, such that the PEGylated aptamer preferentially accumulates in inflamed tissue. In some instances, the 20 kDa PEGylated aptamer conjugate is able to access the interior of cells, such as, for example, kidney cells.

Modified nucleotides can also be used to modulate the plasma clearance of aptamers. For example, an unconjugated aptamer which incorporates both 2′-F and 2′-OMe stabilizing chemistries, which is typical of current generation aptamers as it exhibits a high degree of nuclease stability in vitro and in vivo, displays rapid loss from plasma (i.e., rapid plasma clearance) and a rapid distribution into tissues, primarily into the kidney, when compared to unmodified aptamer.

### PEG-Derivatized Nucleic Acids

As described above, derivatization of nucleic acids with high molecular weight non-immunogenic polymers has the potential to alter the pharmacokinetic and pharmacodynamic properties of nucleic acids making them more effective therapeutic agents. Favorable changes in activity can include increased resistance to degradation by nucleases, decreased filtration through the kidneys, decreased exposure to the immune system, and altered distribution of the therapeutic through the body.

The aptamer compositions of the invention may be derivatized with polyalkylene glycol (“PAG”) moieties. Examples of PAG-derivatized nucleic acids are found in U.S. patent application Ser. No. 10/718,833, filed on Nov. 21, 2003, which is herein incorporated by reference in its entirety. Typical polymers used in the invention include polyethylene glycol (“PEG”), also known as polyethylene oxide (“PEO”) and polypropylene glycol (including poly isopropylene glycol). Additionally, random or block copolymers of different alkylene oxides (e.g., ethylene oxide and propylene oxide) can be used in many applications. In its most common form, a polyalkylene glycol, such as PEG, is a linear polymer terminated at each end with hydroxyl groups: HO—CH₂CH₂O—(CH₂CH₂O)ₙ—CH₂CH₂—OH. This polymer, alpha-, omega-dihydroxylpolyethylene glycol, can also be represented as HO-PEG-OH, where it is understood that the -PEG- symbol represents the following structural unit: —CH₂CH₂O—(CH₂CH₂O)ₙ—CH₂CH₂— where n typically ranges from about 4 to about 10,000.

As shown, the PEG molecule is di-functional and is sometimes referred to as “PEG diol.” The terminal portions of the PEG molecule are relatively non-reactive hydroxyl moieties, the —OH groups, that can be activated, or converted to functional moieties, for attachment of the PEG to other compounds at reactive sites on the compound. Such activated PEG diols are referred to herein as bi-activated PEGs. For example, the terminal moieties of PEG diol have been functionalized as active carbonate ester for selective reaction with amino moieties by substitution of the relatively non-reactive hydroxyl moieties, —OH, with succinimidyl active ester moieties from N-hydroxy succinimide.

In many applications, it is desirable to cap the PEG molecule on one end with an essentially non-reactive moiety so that the PEG molecule is mono-functional (or mono-activated). In the case of protein therapeutics which generally display multiple reaction sites for activated PEGs, bi-functional activated PEGs lead to extensive cross-linking, yielding poorly functional aggregates. To generate mono-activated PEGs, one hydroxyl moiety on the terminus of the PEG diol molecule typically is substituted with non-reactive methoxy end moiety, —OCH₃. The other, un-capped terminus of the PEG molecule typically is converted to a reactive end moiety that can be activated for attachment at a reactive site on a surface or a molecule such as a protein.

PAGs are polymers which typically have the properties of solubility in water and in many organic solvents, lack of toxicity, and lack of immunogenicity. One use of PAGs is to covalently attach the polymer to insoluble molecules to make the resulting PAG-molecule “conjugate” soluble. For example, it has been shown that the water-insoluble drug paclitaxel, when coupled to PEG, becomes water-soluble. Greenwald, et al., J. Org. Chem., 60:331-336 (1995). PAG conjugates are often used not only to enhance solubility and stability but also to prolong the blood circulation half-life of molecules.

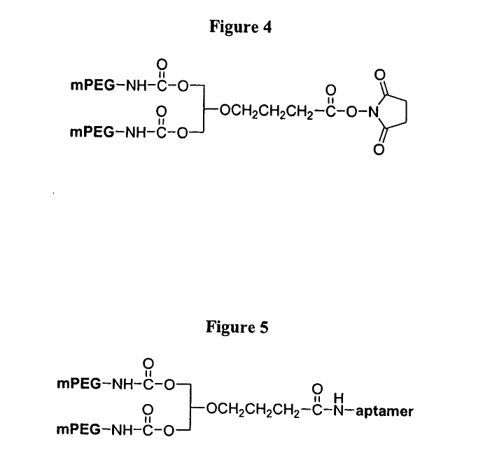

Polyalkylated compounds of the invention are typically between 5 and 80 kDa in size however any size can be used, the choice dependent on the aptamer and application. Other PAG compounds of the invention are between 10 and 80 kDa in size. Still other PAG compounds of the invention are between 10 and 60 kDa in size. For example, a PAG polymer may be at least 10, 20, 30, 40, 50, 60, or 80 kDa in size. Such polymers can be linear or branched. In some embodiments the polymers are PEG. In some embodiment the polymers are branched PEG. In still other embodiments the polymers are 40 kDa branched PEG as depicted in FIG. 4. In some embodiments the 40 kDa branched PEG is attached to the 5′ end of the aptamer as depicted in FIG. 5.

In contrast to biologically-expressed protein therapeutics, nucleic acid therapeutics are typically chemically synthesized from activated monomer nucleotides. PEG-nucleic acid conjugates may be prepared by incorporating the PEG using the same iterative monomer synthesis. For example, PEGs activated by conversion to a phosphoramidite form can be incorporated into solid-phase oligonucleotide synthesis. Alternatively, oligonucleotide synthesis can be completed with site-specific incorporation of a reactive PEG attachment site. Most commonly this has been accomplished by addition of a free primary amine at the 5′-terminus (incorporated using a modifier phosphoramidite in the last coupling step of solid phase synthesis). Using this approach, a reactive PEG (e.g., one which is activated so that it will react and form a bond with an amine) is combined with the purified oligonucleotide and the coupling reaction is carried out in solution.

The ability of PEG conjugation to alter the biodistribution of a therapeutic is related to a number of factors including the apparent size (e.g., as measured in terms of hydrodynamic radius) of the conjugate. Larger conjugates (>10 kDa) are known to more effectively block filtration via the kidney and to consequently increase the serum half-life of small macromolecules (e.g., peptides, antisense oligonucleotides). The ability of PEG conjugates to block filtration has been shown to increase with PEG size up to approximately 50 kDa (further increases have minimal beneficial effect as half life becomes defined by macrophage-mediated metabolism rather than elimination via the kidneys).

Production of high molecular weight PEGs (>10 kDa) can be difficult, inefficient, and expensive. As a route towards the synthesis of high molecular weight PEG-nucleic acid conjugates, previous work has been focused towards the generation of higher molecular weight activated PEGs. One method for generating such molecules involves the formation of a branched activated PEG in which two or more PEGs are attached to a central core carrying the activated group. The terminal portions of these higher molecular weight PEG molecules, i.e., the relatively non-reactive hydroxyl (—OH) moieties, can be activated, or converted to functional moieties, for attachment of one or more of the PEGs to other compounds at reactive sites on the compound. Branched activated PEGs will have more than two termini, and in cases where two or more termini have been activated, such activated higher molecular weight PEG molecules are referred to herein as, multi-activated PEGs. In some cases, not all termini in a branch PEG molecule are activated. In cases where any two termini of a branch PEG molecule are activated, such PEG molecules are referred to as bi-activated PEGs. In some cases where only one terminus in a branch PEG molecule is activated, such PEG molecules are referred to as mono-activated. As an example of this approach, activated PEG prepared by the attachment of two monomethoxy PEGs to a lysine core which is subsequently activated for reaction has been described (Harris et al., Nature, vol. 2: 214-221, 2003).

The present invention provides another cost effective route to the synthesis of high molecular weight PEG-nucleic acid (preferably, aptamer) conjugates including multiply PEGylated nucleic acids. The present invention also encompasses PEG-linked multimeric oligonucleotides, e.g., dimerized aptamers. The present invention also relates to high molecular weight compositions where a PEG stabilizing moiety is a linker which separates different portions of an aptamer, e.g., the PEG is conjugated within a single aptamer sequence, such that the linear arrangement of the high molecular weight aptamer composition is, e.g., nucleic acid-PEG-nucleic acid (-PEG-nucleic acid)ₙ where n is greater than or equal to 1.

High molecular weight compositions of the invention include those having a molecular weight of at least 10 kDa. Compositions typically have a molecular weight between 10 and 80 kDa in size. High molecular weight compositions of the invention are at least 10, 20, 30, 40, 50, 60, or 80 kDa in size.

A stabilizing moiety is a molecule, or portion of a molecule, which improves pharmacokinetic and pharmacodynamic properties of the high molecular weight aptamer compositions of the invention. In some cases, a stabilizing moiety is a molecule or portion of a molecule which brings two or more aptamers, or aptamer domains, into proximity, or provides decreased overall rotational freedom of the high molecular weight aptamer compositions of the invention. A stabilizing moiety can be a polyalkylene glycol, such a polyethylene glycol, which can be linear or branched, a homopolymer or a heteropolymer. Other stabilizing moieties include polymers such as peptide nucleic acids (PNA). Oligonucleotides can also be stabilizing moieties; such oligonucleotides can include modified nucleotides, and/or modified linkages, such as phosphorothioates. A stabilizing moiety can be an integral part of an aptamer composition, i.e., it is covalently bonded to the aptamer.

Compositions of the invention include high molecular weight aptamer compositions in which two or more nucleic acid moieties are covalently conjugated to at least one polyalkylene glycol moiety. The polyalkylene glycol moieties serve as stabilizing moieties. In compositions where a polyalkylene glycol moiety is covalently bound at either end to an aptamer, such that the polyalkylene glycol joins the nucleic acid moieties together in one molecule, the polyalkylene glycol is said to be a linking moiety. In such compositions, the primary structure of the covalent molecule includes the linear arrangement nucleic acid-PAG-nucleic acid. One example is a composition having the primary structure nucleic acid-PEG-nucleic acid. Another example is a linear arrangement of: nucleic acid-PEG-nucleic acid-PEG-nucleic acid.

To produce the nucleic acid-PEG-nucleic acid conjugate, the nucleic acid is originally synthesized such that it bears a single reactive site (e.g., it is mono-activated). In a preferred embodiment, this reactive site is an amino group introduced at the 5′-terminus by addition of a modifier phosphoramidite as the last step in solid phase synthesis of the oligonucleotide. Following deprotection and purification of the modified oligonucleotide, it is reconstituted at high concentration in a solution that minimizes spontaneous hydrolysis of the activated PEG. In a preferred embodiment, the concentration of oligonucleotide is 1 mM and the reconstituted solution contains 200 mM NaHCO₃-buffer, pH 8.3. Synthesis of the conjugate is initiated by slow, step-wise addition of highly purified bi-functional PEG. In a preferred embodiment, the PEG diol is activated at both ends (bi-activated) by derivatization with succinimidyl propionate. Following reaction, the PEG-nucleic acid conjugate is purified by gel electrophoresis or liquid chromatography to separate fully-, partially-, and un-conjugated species. Multiple PAG molecules concatenated (e.g., as random or block copolymers) or smaller PAG chains can be linked to achieve various lengths (or molecular weights). Non-PAG linkers can be used between PAG chains of varying lengths.

The 2′-O-methyl, 2′-fluoro and other modified nucleotide modifications stabilize the aptamer against nucleases and increase its half life in vivo. The 3′-3′-dT cap also increases exonuclease resistance. See, e.g., U.S. Pat. Nos. 5,674,685; 5,668,264; 6,207,816; and 6,229,002, each of which is incorporated by reference herein in its entirety.

### PAG-Derivatization of a Reactive Nucleic Acid

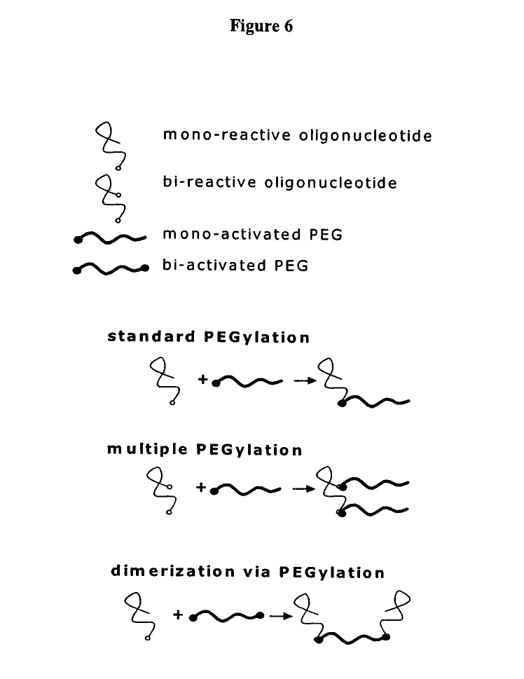

High molecular weight PAG-nucleic acid-PAG conjugates can be prepared by reaction of a mono-functional activated PEG with a nucleic acid containing more than one reactive site. In one embodiment, the nucleic acid is bi-reactive, or bi-activated, and contains two reactive sites: a 5′-amino group and a 3′-amino group introduced into the oligonucleotide through conventional phosphoramidite synthesis, for example: 3′-5′-di-PEGylation as illustrated in FIG. 6. In alternative embodiments, reactive sites can be introduced at internal positions, using for example, the 5-position of pyrimidines, the 8-position of purines, or the 2′-position of ribose as sites for attachment of primary amines. In such embodiments, the nucleic acid can have several activated or reactive sites and is said to be multiply activated. Following synthesis and purification, the modified oligonucleotide is combined with the mono-activated PEG under conditions that promote selective reaction with the oligonucleotide reactive sites while minimizing spontaneous hydrolysis. In the preferred embodiment, monomethoxy-PEG is activated with succinimidyl propionate and the coupled reaction is carried out at pH 8.3. To drive synthesis of the bi-substituted PEG, stoichiometric excess PEG is provided relative to the oligonucleotide. Following reaction, the PEG-nucleic acid conjugate is purified by gel electrophoresis or liquid chromatography to separate fully, partially, and un-conjugated species.

The linking domains can also have one or more polyalkylene glycol moieties attached thereto. Such PAGs can be of varying lengths and may be used in appropriate combinations to achieve the desired molecular weight of the composition.

The effect of a particular linker can be influenced by both its chemical composition and length. A linker that is too long, too short, or forms unfavorable steric and/or ionic interactions with the IL-23 and/or IL-12 will preclude the formation of complex between the aptamer and IL-23 and/or IL-12. A linker, which is longer than necessary to span the distance between nucleic acids, may reduce binding stability by diminishing the effective concentration of the ligand. Thus, it is often necessary to optimize linker compositions and lengths in order to maximize the affinity of an aptamer to a target.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

### Example 1

### Aptamer Selection and Sequences

### IL-23 Aptamer Selection

Several SELEX™ strategies were employed to generate ligands with a variety of specificities for IL-23 and IL-12. One scheme, designed to produce aptamers specific for IL-23 vs. IL-12, included IL-12 in a negative selection step to eliminate aptamers that recognize the common subunit and select for aptamers specific to IL-23. A separate SELEX™ scheme in which IL-23 and IL-12 were alternated every other round elicited aptamers that recognized the common subunit and therefore recognized both proteins. In Examples 1A and 1E, selections were done with 2′-OH purine and 2′-F pyrimidine (rRfY) containing pools. Clones from these selections were optimized based on their binding affinity and efficacy in blocking IL-23 activity in a cell based assay. In addition, selections with 2′-OMe nucleotide containing pools, i.e., rRmY (2′-OH A and G, and 2′-OMe C and U), rGmH (2′-OH G and 2′-OMe C, U, A), and dRmY (deoxy A and G, and 2′-OMe C and U) are described in Examples 1B, 1C, and 1D below.

### Example 1A

### Selections Against Human IL-23 with 2′-Fluoro Pyrimidines Containing Pools (rRfY)

Three selections were performed to identify aptamers to human (“h”)-IL-23 using a pool consisting of 2′-OH purine (ribo-purines) and 2′-F pyrimidine nucleotides (rRfY conditions). The first selection (h-IL-23) was a direct selection against h-IL-23, which is comprised of p19 and p40 domains. The second selection (X-IL-23) utilized h-IL-23 and h-IL-12 in alternating rounds to drive selection of aptamers to the common subunit between the two proteins, p40. In the third selection (PN-IL-23), h-IL-12 was included in the negative selection step to drive enrichment of aptamers binding to the subdomain unique to h-IL-23, p19. As described below, the starting material for this third selection, i.e., the PN-IL-23 selection was a portion of the pool from the h-IL-23 selection, separated from the remainder of the h-IL-23 pool after two rounds of selection against h-IL-23 protein. All three selection strategies yielded aptamers to h-IL-23. Several aptamers are highly specific for h-IL-23, several show cross reactivity between h-IL-23 and h-IL-12, and one is more specific for h-IL-12 vs. h-IL-23.

Round 1 of the h-IL-23 and the PN-IL-23 selection began with incubation of 2×014 molecules of 2° F. pyrimidine modified ARC 212 pool (SEQ ID NO 8) (5′gggaaaagcgaaucauacacaaga-N40-gcuccgccagagaccaaccgagaa3′), including a spike of α³²P ATP body labeled pool, with 100 pmoles of IL-23 protein (R&D, Minneapolis, Minn.) in a final volume of 100 μL for 1 hr at room temperature. The series of N's in the template (SEQ ID NO 8) can be any combination of nucleotides and gives rise to the unique sequence region of the resulting aptamers.

After Round 2, the pool was divided into two equal portions, one portion was used for subsequent rounds (i.e., Rounds 3-12) of the h-IL-23 selection and the other portion was used for the subsequent rounds (i.e., Rounds 3-11) of the PN-IL-23 selection. Round 1 of the X-IL-23 selection was conducted similarly, except the pool RNA was incubated with 50 pmoles of h-IL-23 and 50 pmoles of h-IL-12.

All selections were performed in 1×SHMCK buffer, pH 7.4 (20 mM Hepes pH 7.4, 120 mM NaCl, 5 mM KCl, 1 mM MgCl₂, 1 mM CaCl₂). RNA:h-IL-23 complexes and free RNA molecules were separated using 0.45 μm nitrocellulose spin columns from Schleicher & Schuell (Keene, N.H.). The columns were pre-washed with 1 mL 1×SHMCK, and then the RNA:protein containing solutions were added to the columns and spun in a centrifuge at 1500 g for 2 minutes. Buffer washes were performed to remove nonspecific binders from the filters (Round 1, 2×500 μL 1×SHMCK; in later rounds, more stringent washes of increased number and volume to enrich for specific binders), then the RNA:protein complexes attached to the filters were eluted with 2×200 μL washes (2×100 μL washes in later rounds) of elution buffer (7 M urea, 100 mM sodium acetate, 3 mM EDTA, pre-heated to 95° C.). The eluted RNA was phenol:chloroform extracted, then precipitated (40 μg glycogen, 1 volume isopropanol). The RNA was reverse transcribed with the Thermoscript™ RT-PCR system (Invitrogen, Carlsbad, Calif.) according to the manufacturer's instructions, using the 3′ primer 5′ttctcggttggtctctggcggagc 3′ (SEQ ID NO 10), followed by amplification by PCR (20 mM Tris pH 8.4, 50 mM KCl, 2 mM MgCl₂, 0.5 μM of 5′ primer 5′taatacgactcactatagggaaaagcgaatcatacacaaga 3′ (SEQ ID NO 9), 0.5 μM of 3′ primer (SEQ ID NO 10), 0.5 mM each dNTP, 0.05 units/μL Taq polymerase (New England Biolabs, Beverly, Mass.)). PCR reactions were done under the following cycling conditions: a) 94° C. for 30 seconds; b) 55° C. for 30 seconds; c) 72° C. for 30 seconds. The cycles were repeated until sufficient PCR product was generated. The minimum number of cycles required to generate sufficient PCR product is reported in Tables 1-3 below as the “PCR Threshold”.

The PCR templates were purified using the QIAquick PCR purification kit (Qiagen, Valencia, Calif.). Templates were transcribed using α₃₂P ATP body labeling overnight at 37° C. (4% PEG-8000, 40 mM Tris pH 8.0, 12 mM MgCl₂, 1 mM spermidine, 0.002% Triton X-100, 3 mM 2′OH purines, 3 mM 2° F. pyrimidines, 25 mM DTT, 0.0025 units/μL inorganic pyrophosphatase, 2 μg/mL T7 Y639F single mutant RNA polymerase, 5 μCi α³²P ATP). The reactions were desalted using Bio Spin columns (Bio-Rad, Hercules, Calif.) according to the manufacturer's instructions.

Subsequent rounds of all three selections were repeated using the same method as for Round 1, except for the changes indicated in Tables 1-3. Prior to incubation with protein target, the pool RNA was passed through a 0.45 micron nitrocellulose filter column to remove filter binding sequences, then the filtrate was carried on into the positive selection step. In alternating rounds the pool RNA was gel purified. Transcription reactions were quenched with 50 mM EDTA and ethanol precipitated then purified on a 1.5 mm denaturing polyacrylamide gel (8 M urea, 10% acrylamide; 19:1 acrylamide:bisacrylamide). Pool RNA was removed from the gel by electroelution in an Elutrap® apparatus (Schleicher and Schuell, Keene, N.H.) at 225V for 1 hour in 1×TBE (90 mM Tris, 90 mM boric acid, 0.2 mM EDTA). The eluted material was precipitated by the addition of 300 mM sodium acetate and 2.5 volumes of ethanol.

The RNA remained in excess of the protein throughout the selections (˜1-2 μM RNA). The protein concentration was 1 μM for the first 2 rounds, and then was dropped to varying lower concentrations based on the particular selection. Competitor tRNA was added to the binding reactions at 0.1 mg/mL starting at Round 3 or 4, depending on the selection. A total of 11-12 rounds were completed, with binding assays performed at select rounds. Tables 1-3 below contains the selection details used for the rRfY selections using the h-IL-23, X-IL-23, and PN-IL-23 selection strategies; including pool RNA concentration, protein concentration, and tRNA concentration used for each round. Elution values (ratio of CPM values of protein-bound RNA versus total RNA flowing through the filter column) along with dot blot binding assays were used to monitor selection progress.

TABLE 1
Conditions used for h-IL-23 Selection

| | RNA | | | tRNA | | | |
|---|---|---|---|---|---|---|---|
| | pool | | protein | conc | | | |
| Round | conc | protein | conc | (mg/ | | | PCR |
| # | (μM) | type | (μM) | mL) | neg | % elution | Threshold |
| 1 | 3.3 | IL-23 | 1 | 0 | none | 4.38 | 10 |
| 2 | ~1 | IL-23 | 1 | 0 | NC | 0.85 | 10 |
| 3 | 0.8 | IL-23 | 0.75 | 0 | NC | 10.9 | 8 |
| 4 | ~1 | IL-23 | 0.5 | 0.1 | NC | 0.53 | 8 |
| 5 | 1 | IL-23 | 0.1 | 0.1 | NC | 1.72 | 11 |
| 6 | ~1 | IL-23 | 0.1 | 0.1 | NC | 0.11 | 12 |
| 7 | 1 | IL-23 | 0.1 | 0.1 | NC | 1.15 | 8 |
| 8 | ~0.5 | IL-23 | 0.05 | 0.1 | NC | 0.12 | 11 |
| 9 | 0.5 | IL-23 | 0.05 | 0.1 | NC | 3.54 | 8 |
| 10 | ~0.5 | IL-23 | 0.05 | 0.1 | NC | 0.18 | 12 |
| 11 | 0.5 | IL-23 | 0.025 | 0.1 | NC | 1.09 | 12 |
| 12 | ~0.5 | IL-23 | 0.025 | 0.1 | NC | 0.07 | 12 |

TABLE 2
Conditions used for X-IL-23 Selection

| | RNA | | | tRNA | | | |
|---|---|---|---|---|---|---|---|
| | pool | | protein | conc | | | |
| Round | conc | protein | conc | (mg/ | | | PCR |
| # | (μM) | type | (μM) | mL) | neg | % elution | Threshold |
| 1 | 3.3 | IL-23/ | 0.5 | 0 | none | 3.15 | 10 |
| | | IL-12 | each | | | | |
| 2 | ~1 | IL-23/ | 0.5 | 0 | NC | 0.56 | 10 |
| | | IL-12 | each | | | | |
| 3 | 0.8 | IL-12 | 0.75 | 0 | NC | 0.58 | 13 |
| 4 | ~1 | IL-23 | 0.75 | 0.1 | NC | 0.37 | 8 |
| 5 | 1 | IL-12 | 0.5 | 0.1 | NC | 0.38 | 11 |
| 6 | ~1 | IL-23 | 0.1 | 0.1 | NC | 0.08 | 12 |
| 7 | 1 | IL-12 | 0.1 | 0.1 | NC | 0.50 | 9 |
| 8 | ~0.5 | IL-23 | 0.05 | 0.1 | NC | 0.10 | 11 |
| 9 | 0.5 | IL-12 | 0.05 | 0.1 | NC | 0.83 | 11 |
| 10 | ~0.5 | IL-23 | 0.05 | 0.1 | NC | 0.17 | 8 |
| 11 | 0.5 | IL-12 | 0.025 | 0.1 | NC | 0.91 | 12 |
| 12 | ~0.5 | IL-23 | 0.025 | 0.1 | NC | 0.05 | 12 |

TABLE 3
Conditions used for PN-IL-23

| | | | | | | neg | | |
|---|---|---|---|---|---|---|---|---|
| | RNA | | | | | IL- | | |
| | pool | | protein | tRNA | | 12 | | |
| | conc | protein | conc | conc | | conc | | PCR |
| Round # | (μM) | type | (μM) | (mg/mL) | neg | (μM) | % elution | Threshold |
| 1 | 3.3 | IL-23 | 1 | 0 | none | 0 | 4.38 | 10 |
| 2 | ~1 | IL-23 | 1 | 0 | NC | 0 | 0.85 | 10 |
| 3 | 0.8 | IL-23 | 0.75 | 0.1 | NC/IL-12 | 0.75 | 1.15 | 10 |
| 4 | ~1 | IL-23 | 0.75 | 0.1 | NC/IL-12 | 0.75 | 0.59 | 10 |
| 5 | 0.7 | IL-23 | 0.5 | 0.1 | NC/IL-12 | 0.5 | 4.19 | 10 |
| 6 | ~1 | IL-23 | 0.1 | 0.1 | NC/IL-12 | 0.5 | 0.05 | 14 |
| 7 | 1 | IL-23 | 0.1 | 0.1 | NC/IL-12 | 0.5 | 0.38 | 10 |
| 8 | ~1 | IL-23 | 0.1 | 0.1 | NC/IL-12 | 0.3 | 0.18 | 15 |
| 9 | 1 | IL-23 | 0.1 | 0.1 | NC/IL-12 | 0.5 | 2.81 | 8 |
| 10 | ~1 | IL-23 | 0.05 | 0.1 | NC/IL-12 | 0.5 | 0.21 | 10 |
| 11 | ~1 | IL-23 | 0.05 | 0.1 | NC/IL-12 | 0.5 | 1.35 | 12 |

Monitoring Progress of rRfY Selection. Dot blot binding assays were performed throughout the selections to monitor the protein binding affinity of the pools. Trace ³²P-labeled RNA was combined with a dilution series of h-IL-23 and incubated at room temperature for 30 minutes in 1×SHMCK (20 mM Hepes, 120 mM NaCl, 5 mM KCl, 1 mM MgCl₂, 1 mM CaCl₂, pH 7.4) plus 0.1 mg/mL tRNA for a final volume of 20 μL. The binding reactions were analyzed by nitrocellulose filtration using a Minifold I dot-blot, 96-well vacuum filtration manifold (Schleicher & Schuell, Keene, N.H.). A three-layer filtration medium was used, consisting (from top to bottom) of Protran nitrocellulose (Schleicher & Schuell), Hybond-P nylon (Amersham Biosciences) and GB002 gel blot paper (Schleicher & Schuell). RNA that is bound to protein is captured on the nitrocellulose filter, whereas the non-protein bound RNA is captured on the nylon filter. The gel blot paper was included simply as a supporting medium for the other filters. Following filtration, the filter layers were separated, dried and exposed on a phosphor screen (Amersham Biosciences, Piscataway, N.J.) and quantified using a Storm 860 Phosphorimager® blot imaging system (Amersham Biosciences).

When a significant positive ratio of binding of RNA in the presence of h-IL-23 versus in the absence of h-IL-23 was seen, the pools were cloned using a TOPO TA cloning kit (Invitrogen, Carlsbad, Calif.) according to the manufacturer's instructions. For the h-IL-23 and X-IL-23 selections, the Round 8 pool templates were cloned, and 32 individual clones from each selection were assayed in a 1-point dot blot screen (+/−75 nM h-IL-23, as well as a separate screen at +/−75 nM h-IL-12). For the PN-IL-23 selection, the Round 10 pool was cloned and sequenced, and 8 unique clones were assayed for protein binding in a 1-point dot blot screen (+/−200 nM h-IL-23 and a separate screen at +/−200 nM h-IL-12). Subsequently, the Round 10 PN-IL-23 pool was re-cloned for further sequences, as well as the R12 PN-IL-23 pool, and the clones were assayed for protein binding in a 1 point do blot screen (+/−100 nM h-IL-23 or +/−200 nM h-IL-12). For K_{D} determination, the clone transcripts were 5′ end labeled with γ-³²P ATP. K_{D} values were determined using a dilution series of h-IL-23 (R&D Systems, Minneapolis, Minn.) in the dot blot assay for all unique sequences with good +/−h-IL-23 binding ratios in the initial screens, and fitting an equation describing a 1:1 RNA:protein complex to the resulting data (fraction aptamer bound=amplitude*([IL-23]/(K_{D}+[IL-23])) (KaleidaGraph v. 3.51, Synergy Software). Results of protein binding characterization are tabulated in Table 4. Clones with high affinity to h-IL-23 were prepped and screened for functionality in cell-based assays, described in Example 3 below.

TABLE 4
rRfY Clone binding activity (all measurements were made in the presence
of 0.1 mg/mL tRNA)

| Round 8 h-IL-23 | | | | | 1-pt Screen Data | |
|---|---|---|---|---|---|---|
| SEQ | Clone | K_{D}IL-23 | K_{D} IL-12 | K_{D} IL-12/K_{D} | +/−IL-23 | +/−IL-12 |
| ID NO | Name | (nM) | (nM) | IL-23 | 75 nM | 75 nM |
| 15 | AMX86-B5 | 195.5 | N.B. | | 5.79 | 1.01 |
| 27 | AMX86-C5 | 80.3 | 399.8 | 4.98 | 6.23 | 2.65 |
| 13 | AMX86-D5 | 27.4 | N.B. | | 7.17 | 1.52 |
| 16 | AMX86-D6 | 25 | N.B. | | 9.82 | 1.43 |
| 24 | AMX86-E6 | 51.3 | N.B. | | 9.02 | 1.13 |
| 22 | AMX86-F6 | 69.1 | N.B. | | 10.17 | 1.36 |
| 18 | AMX86-A7 | 57.7 | 667.9 | 11.58 | 3.99 | 1.59 |
| 14 | AMX86-B7 | 111 | 934.1 | 8.42 | 7.81 | 1.46 |
| 20 | AMX86-C7 | 140.3 | N.B. | | 4.65 | 0.77 |
| 19 | AMX86-E7 | 210.2 | 267.5 | 1.27 | 6.79 | 1.23 |
| 21 | AMX86-F7 | 147 | 106.4 | 0.72 | 13.07 | 2.49 |
| 25 | AMX86-H7 | 89.8 | N.B. | | 10.85 | 1.26 |
| 26 | AMX86-C8 | 107.1 | N.B. | | 5.28 | 1.17 |
| 23 | AMX86-D8 | 294.2 | N.B. | | 6.87 | 1.08 |
| 17 | AMX86-G8 | 133.7 | 2493.1 | 18.65 | 7.26 | 2.05 |

| | | | | | 1-pt | |
|---|---|---|---|---|---|---|
| Round 8 X-IL-23 | | IL-23 | | K_{D} IL- | Screen Data | |
| SEQ ID | | K_{D} | IL-12 K_{D} | 12/K_{D} IL- | +/−IL-23 | +/−IL-12 |
| NO | Clone Name | (nM) | (nM) | 23 | 75 nM | 75 nM |
| 41 | AMX86-A9 | 190.5 | N.B. | | 3.55 | 0.68 |
| 35 | AMX86-B9 | 23.7 | 847.6 | 35.76 | 12.88 | 1.96 |
| 32 | AMX86-C9 | 97.9 | 672.8 | 6.87 | 6.07 | 1.86 |
| 33 | AMX86-G9 | 109.4 | N.B. | | 10.03 | 1.04 |
| 39 | AMX86-H9 | 104.6 | 331.5 | 3.17 | 10.35 | 3.66 |
| 34 | AMX86-A10 | 460.9 | 289.4 | 0.63 | 6.64 | 1.40 |
| 28 | AMX86-B10 | 77.8 | 1038.3 | 13.35 | 4.73 | 2.12 |
| 42 | AMX86-E10 | 218.1 | 904.6 | 4.15 | 2.44 | 1.37 |
| 36 | AMX86-G10 | 73.7 | 356.1 | 4.83 | 9.88 | 2.41 |
| 37 | AMX86-A11 | 157.2 | 182.4 | 1.16 | 7.05 | 3.23 |
| 29 | AMX86-B11 | 179.9 | 5950 | 33.07 | 9.23 | 1.69 |
| 30 | AMX86-D11 | 198.9 | 113.9 | 0.57 | 10.26 | 2.59 |
| 38 | AMX86-F11 | 255.64 | 540.6 | 2.11 | 7.33 | 2.87 |
| 40 | AMX86-H11 | 366.9 | 214.9 | 0.59 | 7.56 | 3.02 |
| 31 | AMX86-F12 | 423.7 | 2910.3 | 6.87 | 11.88 | 2.51 |

| PN-IL-23 Clones | | PN-IL- | | IL-12 | 1-pt Screen Data | | |
|---|---|---|---|---|---|---|---|
| SEQ | | 23 | IL-23 | K_{D} | +/−IL-23 | +/−IL-23 | +/−IL-12 |
| ID NO | Clone Name | Round | K_{D} (nM) | (nM) | 200 nM | 100 nM | 200 nM |
| 43 | AMX 84-A10 | R10 | 22.3 | N.B. | 39.6 | | 2.9 |
| 44 | AMX 84-B10 | R10 | 21.8 | N.B. | 22.7 | | 1.3 |
| 45 | AMX 84-A11 | R10 | 17.8 | N.B. | 32.7 | | 1.8 |
| 46 | AMX 84-F11 | R10 | 16.6 | N.B. | 22.5 | | 0.8 |
| 47 | AMX 84-E12 | R10 | 27.8 | N.B. | 15.8 | | 0.8 |
| 48 | AMX 84-C10 | R10 | 94.3 | N.B. | 17.7 | | 2.2 |
| 49 | AMX 84-C11 | R10 | 15.5 | 286.1 | 23.4 | | 2.7 |
| 50 | AMX 84-G11 | R10 | 290.7 | N.B. | 22.3 | | 1.7 |
| 51 | ARX33-plate1- | R12 | 77.8 | N.B. | | 20.3 | 1.7 |
| | H01 | | | | | | |
| 52 | AMX 91-F11 | R10 | 201.7 | N.B. | | 11.4 | 2.2 |
| 53 | AMX 91-G1 | R10 | 82.3 | N.B. | | 52.2 | 1.7 |
| 54 | AMX 91-E3 | R10 | 205.3 | N.B. | | 34.4 | 2.9 |
| 55 | AMX 91-H3 | R10 | 265.7 | N.B. | | 18.5 | 2.3 |
| 56 | AMX 91-B5 | R10 | 148.5 | N.B. | | 11.2 | 0.9 |
| 57 | AMX 91-A6 | R10 | 60.3 | N.B. | | 6.3 | 1.1 |
| 58 | AMX 91-G7 | R12 | 63.6 | N.B. | | 38.1 | 1.9 |
| 59 | AMX 91-H7 | R12 | 71.0 | N.B. | | 44.7 | 1.4 |
| 60 | AMX 91-B8 | R12 | 17.6 | 409.1 | | 34.0 | 7.9 |
| 61 | AMX 91-H8 | R12 | 16.6 | 243.2 | | 25.2 | 4.1 |
| 62 | AMX 91-G9 | R12 | 33.0 | N.B. | | 31.7 | 1.1 |
| 63 | AMX 91-D9 | R12 | 44.6 | N.B. | | 25.1 | 2.1 |
| 64 | AMX 91-G11 | R12 | 104.4 | N.B. | | 12.5 | 1.7 |
| 65 | AMX 91-C12 | R12 | 30.7 | N.B. | | 22.9 | 1.9 |
| 66 | AMX 91-H12 | R12 | 60.8 | N.B. | | 48.6 | 1.2 |

N.B. = no significant binding observed

The nucleic acid sequences of the rRfY aptamers characterized in Table 5 are given below. The unique sequence of each aptamer below begins at nucleotide 25, immediately following the sequence GGGAAAAGCGAAUCAUACACAAGA (SEQ ID NO 11) and runs until it meets the 3′fixed nucleic acid sequence GCUCCGCCAGAGACCAACCGAGAA (SEQ ID NO 12).

Unless noted otherwise, individual sequences listed below are represented in the 5′ to 3′ orientation and represent the sequences that bind to IL-23 and/or IL-12 selected under rRfY SELEX™ conditions wherein the purines (A and G) are 2′-OH and the pyrimidines (U and C) are 2′-fluoro. Each of the sequences listed in Table 5 may be derivatized with polyalkylene glycol (“PAG”) moieties and may or may not contain capping (e.g., a 3′-inverted dT).

TABLE 5

rRfY Clone sequences from h-IL-23 Selection
(Round 8), X-IL-23 Selection (round 8), PN-IL-23
Selection (Round10/12).

h-IL-23 Selection (Round 8)
SEQ ID NO 13 (AMX(86)-D5)
GGGAAAAGCGAAUCAUACACAAGAGAGGUAUGUGGUUUUGCGGAGCAACU
CGUGUCAGCGGUCAGCUCCGCCAGAGACCAACCGAGAA
SEQ ID NO 14 (AMX(86)-B7)
GGGAAAAGCGAAUCAUACACAAGAAUGAAUUCCGUCCACGGGCGCCCGAU
GAUGUCAGUUUUCGGCUCCGCCAGAGACCAACCGAGAA
SEQ ID NO 15 (AMX(86)-B5)
GGGAAAAGCGAAUCAUACACAAGAUUAGUGCGUGUGUUGAAAGGGCUCAU
AAUGUCAGUAUCGAGCUCCGCCAGAGACCAACCGAGAA
SEQ ID NO 16 (AMX(86)-D6)
GGGAAAAGCGAAUCAUACACAAGAUUAGGCGUCGUGACAAUAACUGGUGC
ACGAGCAUGUCAGUGCUCCGCCAGAGACCAACCGAGAA
SEQ ID NO 17 (AMX(86)-G8)
GGGAAAAGCGAAUCAUACACAAGAUGGAAGGCGAUCGUAGCAGUAACCCA
AUGAUUGGGACCUAGCUCCGCCAGAGACCAACCGAGAA
SEQ ID NO 18 (AMX(86)-A7)
GGGAAAAGCGAAUCAUACACAAGAUCUCUUUGGCCGACGCAACAAUGCUC
UUUUCCGACCUUGCGCUCCGCCAGAGACCAACCGAGAA
SEQ ID NO 19 (AMX(86)-E7)
GGGAAAAGCGAAUCCUACCCAAGAUGUUGUUGGCGUUGAUCGUAUGAUUN
AUGGAGNGUGUCNGUGCUCCGCCAGAGACCAACCGAGAA
SEQ ID NO 20 (AMX(86)-C7)
GGGAAAAGCGAAUCAUACACAAGAUGCGCUAUGUUUGGCUGGGAAUUGUA
GCAUUGCUCAAGUGGCUCCGCCAGAGACCAACCGAGAA
SEQ ID NO 21 (AMX(86)-F7)
GGGAAAAGCGAAUCAUACACAAGAUGUUGAACCUCUUGUGCGUCCCGAUG
UUUNGCAAUGUGGAGCUCCGCCAGAGACCAACCGAGAA
SEQ ID NO 22 (AMX(86)-F6)
GGGAAAAGCGAAUCAUACACAAGAAUGUAUACAAUGCCCUAUCGUCAGUU
AGGCAUGUGUGGAUGCUCCGCCAGAGACCAACCGAGAA
SEQ ID NO 23 (AMX(86)-D8)
GGGAAAAGCGAAUCAUACACAAGACAGAGGCAAUGAGAGCCUGGCGAUGU
CAGUCGCAUCUUGCUGCUCCGCCAGAGACCAACCGAGAA
SEQ ID NO 24 (AMX(86)-E6)
GGGAAAAGCGAAUCAUACACAAGAUCGCAAAAGGAGUUUGUCUCUGCUCU
CGGAGUGUGUCAGUGCUCCGCCAGAGACCAACCGAGAA
SEQ ID NO 25 (AMX(86)-H7)
GGGAAAAGCGAAUCAUACACAAGAGAUGACUACACGCCAGUGUGCGCUUU
UUGCGGAGUUAGCGGCUCCGCCAGAGACCAACCGAGAA
SEQ ID NO 26 (AMX(86)-C8)
GGGAAAAGCGAAUCAUACACAAGAGUCGUGAUGAUUUGGGUUAUGUCAGU
UCCCUGUAUGGUUUCGCUCCGCCAGAGACCAACCGAGAA
SEQ ID NO 27 (AMX(86)-C5)
GGGAAAAGCGAAUCAUACACAAGAGUUUUAUGUGGGUCCCGAUGAUUAAC
UUUAUUGGCGCAUUGCUCCGCCAGAGACCAACCGAGAA
X-IL-23 Selection (Round 8)
SEQ ID NO 28 (AMX(86)-B10)
GGGAAAAGCGAAUCAUACACAAGAGAACGAGUAUAUUUGCGCUGGCGGAG
AAGUCUCUCGAAGGGAGCUCCGCCAGAGACCAACCGAGAA
SEQ ID NO 29 (AMX(86)-B11)
GGGAAAAGCGAAUCAUACACAAGAGUAUCAUUCGGCUGGUGGGAGAAAUC
UCUGUAGAUAUAGAGCUCCGCCAGAGACCAACCGAGAA
SEQ ID NO 30 (AMX(86)-D11)
GGGAAAAGCGAAUCAUACACAAGAUAGCGUCUAUGAUGGCGGAGAAGCAA
GUGUAGCAUAACAGGCUCCGCCAGAGACCAACCGAGAA
SEQ ID NO 31 (AMX(86)-F12)
GGGAAAAGCGAAUCAUACACAAGAGUGUUGAAUGAGCGCUGGUGGACAGA
UCUUUGGUUACAGAGCUCCGCCAGAGACCAACCGAGAA
SEQ ID NO 32 (AMX(86)-C9)
GGGAAAAGCGAAUCAUACACAAGACUCAUGGAUAUGGCCUAGCAGCCGUG
GAAGCGGUCAUUCUGCUCCGCCAGAGACCAACCGAGAA
SEQ ID NO 33 (AMX(86)-G9)
GGGAAAAGCGAAUCAUACACAAGAUCCCAGCGGUACGUGAGUCUGUUAAA
GGCCACCUAAUGUCGCUCCGCCAGAGACCAACCGAGAA
SEQ ID NO 34 (AMX(86)-A10)
GGGAAAAGCGAAUCAUACACAAGAGUAAUGUGGGUCCCGAUGAUUCGCUG
UGCGGCGUUUGUAAGCUCCGCCAGAGACCAACCGAGAA
SEQ ID NO 35 (AMX(86)-B9)
GGGAAAAGCGAAUCAUACACAAGAGGUUGAGUACGACGGAGUCNUGGCUA
ACACGGAAACUAGAGCUCCGCCAGAGACCAACCGAGAA
SEQ ID NO 36 (AMX(86)-G10)
GGGAAAAGCGAAUCAUACACAAGAGUCAUGGCUUACAAUUGAAACAAGAG
CUCGCGUGACACAUGCUCCGCCAGAGACCAACCGAGAA
SEQ ID NO 37 (AMX(86)-A11)
GGGAAAAGCGAAUCAUACACAAGAACGGCUAGGCAUCAAUGGCCAGCAAA
AAUAGUCGUGUAAUGCUCCGCCAGAGACCAACCGAGAA
SEQ ID NO 38 (AMX(86)-F11)
GGGAAAAGCGAAUCAUACACAAGACCAUCGGACGAGGCGGGUCACCUUUU
ACGCUUUCGAGCUGGCUCCGCCAGAGACCAACCGAGAA
SEQ ID NO 39 (AMX(86)-H9)
GGGAAAAGCGAAUCAUACACAAGAUGGUUCCCACGUGAAAGUGGCUAGCG
AGUACCCCACUUAUGCUCCGCCAGAGACCAACCAAGGG
SEQ ID NO 40 (AMX(86)-H11)
GGGAAAAGCGAAUCAUACACAAGAGCGCUUUAGCGGGUAUAGCACUUUUC
AUCUAAUGAANCCGUAGCUCCGCCAGAGACCAACCGAGAA
SEQ ID NO 41 (AMX(86)-A9)
GGGAAAAGCGAAUCAUACACAAGAUCUACGAUUGUUCAGGUUUUUUGUAC
UCAACUAAAGGCGAGCUCCGCCAGAGACCAACCGAGAA
SEQ ID NO 42 (AMX(86)-E10)
GGGAAAAGCGAAUCAUACACAAGAUUGUCUCGGAUUGGUCACUCCCAUUU
UUGUUCGCUUAACGGCUCCGCCAGAGACCAACCGAGAA
PN-IL-23 Selection (Round 10 and 12)
SEQ ID NO 43 (AMX(84)-A10)
GGGAAAAGCGAAUCAUACACAAGAAGUUUUUUGUGCUCUGAGUACUCAGC
GUCCGUAAGGGAUAUGCUCCGCCAGAGACCAACCGAGAA
SEQ ID NO 44 (AMX(84)-B10)
GGGAAAAGCGAAUCAUACACAAGAAGUGCUUCAUGCGGCAAACUGCAUGA
CGUCGAAUAGAUAUGCUCCGCCAGAGACCAACCGAGAA
SEQ ID NO 45 (AMX(84)-A11)
GGGAAAAGCGAAUCAUACACAAGAGAGGUAUGUGGUUUUGCGGAGCAACU
CGUGUCAGCGGUCAGCUCCGCCAGAGACCAACCGAGAA
SEQ ID NO 46 (AMX(84)-F11)
GGGAAAAGCGAAUCAUACACAAGAUGUGCUUGAGUUAAAUCUCAUCGUCC
CCGUUUGGGGAUAUGCUCCGCCAGAGACCAACCGAGAA
SEQ ID NO 47 (AMX(84)-E12)
GGGAAAAGCGAAUCAUACACAAGAAGUUUUUGUGCUCUGAGUACUCAGCG
UCCGUAAGGGAUAUGCUCCGCCAGAGACCAACCGAGAA
SEQ ID NO 48 (AMX(84)-C10)
GGGAAAAGCGAAUCAUACACAAGAGAUGUAUUCAGGCGGUCCGCAUUGAU
GUCAGUUAUGCGUAGCUCCGCCAGAGACCAACCGAGAA
SEQ ID NO 49 (AMX(84)-C11)
GGGAAAAGCGAAUCAUACACAAGAAUGGUCGGAAUCUCUGGCGCCACGCU
GAGUAUAGACGGAAGCUCCGCCAGAGACCAACCGAGAA
SEQ ID NO 50 (AMX(84)-G11)
GGGAAAAGCGAAUCAUACACAAGAGUGCUUCGUAUGUUGAAUACGACGUU
CGCAGGACGAAUAUGCUCCGCCAGAGACCAACCGAGAA
SEQ ID NO 51 (ARX33-plate1-H01)
AGGGAAAAGGAAUCAUACACAAGAUGUAUCAUCCGGUCGUACAAAAGCGC
CACGGAACCAUUCGCUCCGCCAGANACCAACCGAGAA
SEQ ID NO 52 (AMX(91)-F11)
GGGAAAAGCGAAUCAUACACAAGACGCGUCAGGUCCACGCUGAAAUUUAU
UUUCGGCAGUGUAAGCUCCGCCAGAGACCAACCGAGAA
SEQ ID NO 53 (AMX(91)-G1)
GGGAAAAGCGAAUCAUACACAAGAUAUGUGCCUGGGAUGGACGACAUCCC
CUGUCUAAGGAUAUGCUCCGCCAGAGACCAACCGAGAA
SEQ ID NO 54 (AMX(91)-E3)
GGGAAAAGCGAAUCAUACACAAGAUUACUCCGUUAGUGUCAGUUGACGGA
GGGAGCGUACUAUUGCUCCGCCAGAGACCAACCGAGAA
SEQ ID NO 55 (AMX(91)-H3)
GGGAAAAGCGAAUCAUACACAAGACAUUGUGCUUUAUCACGUGGGUGAUA
ACGACGAAAGUUAUGCUCCGCCAGAGACCAACCGAGAA
SEQ ID NO 56 (AMX(91)-B5)
GGGAAAAGCGAAUCAUACACAAGACAGUGUAUGAGGAAGAUUACUUCCAU
UCCUGAGCGGUUUUCGCUCCGCCAGAGACCAACCGAGAA
SEQ ID NO 57 (AMX(91)-A6)
GGGAAAAGCGAAUCAUACACAAGAUUGGCAAUGUGACCUUCAACCCUUUU
CCCGAUGAACAGUGGCUCCGCCAGAGACCAACCGAGAA
SEQ ID NO 58 (AMX(91)-G7)
GGGAAAAGCGAAUCAUACACAAGACAUGACUGCAUGCUUCGGGAGUAUCU
CGGUCCCGACGUUCGCUCCGCCAGAGACCAACCGAGAA
SEQ ID NO 59 (AMX(91)-H7)
GGGAAAAGCGAAUCAUACACAAGACUUAUCGCCUCAAGGGGGGUAAUAAA
CCCAGCGUGUGCAUGCUCCGCCAGAGACCAACCGAGAA
SEQ ID NO 60 (AMX(91)-B8)
GGGAAAAGCGAAUCAUACACAAGAAUCCUGGCUUCGCAUAGUGUAUGGGU
AGUACGACAGCGCGUGCUCCGCCAGAGACCAACCGAGAA
SEQ ID NO 61 (AMX(91)-H8)
GGGAAAAGCGAAUCAUACACAAGAACGCAUAGUCGGAUUUACCGAUCAUU
CUGUGCCUUCGUGACGCUCCGCCAGAGACCAACCGAGAA
SEQ ID NO 62 (AMX(91)-G9)
GGGAAAAGCGAAUCAUACACAAGAAUUGUGCUUACAACUUUCGUUGUACC
GACGUGUCAGUUAUGCUCCGCCAGAGACCAACCGAGAA
SEQ ID NO 63 (AMX(91)-D9)
GGGAAAAGCGAAUCAUACACAAGAGUGUAUUACCCCCAACCCAGGGGGAC
CAUUCGCGUAACAAGCUCCGCCAGAGACCAACCGAGAA
SEQ ID NO 64 (AMX(91)-G11)
GGGAAAAGCGAAUCAUACACAAGACUUAACAGUGCGGGGCGCAGUGUAUA
GAUCCGCAAUGUGUGCUCCGCCAGAGACCAACCGAGAA
SEQ ID NO 65 (AMX(91)-C12)
GGGAAAAGCGAAUCAUACACAAGACGAUAGUAUGACCUUUUGAAAGGCUU
CCCGAGCGGUGUUCGCUCCGCCAGAGACCAACCGAGAA
SEQ ID NO 66 (AMX(91)-H12)
GGGAAAAGCGAAUCAUACACAAGACGUGUGCUUUAUGUAAACCAUAACGU
UCCAUAAGGAAUAUGCUCCGCCAGAGACCAACCGAGAA

Those sequences having binding activity to the IL-23 target proteins as determined by the dot blot binding assay described above, and that were functional in cell based assays (described below in Example 3), were minimized (described below in Example 2).

### Example 1B

### IL-23 Selections Against Human IL-23 with Ribo/2′O-Me Nucleotide Containing Pools

Two selections were performed to identify aptamers containing ribo/2′O-Methyl nucleotides. One selection used 2′O-Methyl A, C, and U and 2′OH G (rGmH), and the other selection used 2′-OMe C, U and 2′-OH G, A (rRmY). Both selections were direct selections against h-IL-23 which had been immobilized on a hydrophobic plate. No steps were taken to bias selection of aptamers specific for the p19 or p40 subdomains. Both selections yielded pools significantly enriched for h-IL-23 binding versus naïve, unselected pool. Individual clone sequences are reported herein, and h-IL-23 binding data is provided for selected individual clones.

Pool Preparation. A DNA template with the sequence 5′-GGGAGAGGAGAGAACGTTCTACN₃₀CGCTGTCGATCGATCGATCGATG-3′ (ARC256) (SEQ ID NO 3) was synthesized using an ABI EXPEDITE™ DNA synthesizer, and deprotected by standard methods. The series of N's in the DNA template (SEQ ID NO 3) can be any combination of nucleotides and gives rise to the unique sequence region of the resulting aptamers.

The template was amplified with the 5′ primer 5′-TAATACGACTCACTATAGGGAGAGGAGAGAACGTTCTAC-3′ (SEQ ID NO 67) and 3′ primer 5′-CATCGATCGATCGATCGACAGC-3′ (SEQ ID NO 68) and then used as a template for in vitro transcription with Y639F single mutant T7 RNA polymerase. Transcriptions were done at 37° C. overnight using 200 mM Hepes, 40 mM DTT, 2 mM spermidine, 0.01% Triton X-100, 10% PEG-8000, 5 mM MgCl₂, 1.5 mM MnCl₂, 500 μM NTPs, 500 μM GMP, 0.01 units/mL inorganic pyrophosphatase, and 2 μg/mL Y639F single mutant T7 polymerase. Two different compositions were transcribed, rGmH, and rRmY.

Selection. Each round of selection was initiated by immobilizing 20 pmoles of h-IL-23 to the surface of Nunc Maxisorp hydrophobic plates for 2 hours at room temperature in 100 mL of 1× Dulbecco's PBS (DPBS (+Ca²⁺, Mg²⁺)). The supernatant was then removed and the wells were washed 4 times with 120 μL wash buffer (1×DPBS, 0.2% BSA, and 0.05% Tween-20). Pool RNA was heated to 90° C. for 3 minutes and cooled to room temperature for 10 minutes to refold. In Round 1, a positive selection step was conducted. Briefly, 1×10¹⁴ molecules (0.2 nmoles) of pool RNA were incubated in 100 μL binding buffer (1×DPBS and 0.05% Tween-20) in the wells with immobilized protein target for 1 hour. The supernatant was then removed and the wells were washed 4 times with 120 μL wash buffer. In subsequent rounds a negative selection step was included. The pool RNA was also incubated for 30 minutes at room temperature in empty wells to remove any plastic binding sequences from the pool before the positive selection step. The number of washes was increased after Round 4 to increase stringency. In all cases, the pool RNA bound to immobilized h-IL-23 was reverse transcribed directly in the selection plate by the addition of RT mix (3′ primer, (SEQ ID NO 68), and Thermoscript™ RT, (Invitrogen, Carlsbad, Calif.) followed by incubation at 65° C. for 1 hour.

The resulting cDNA was used as a template for PCR using Taq polymerase (New England Biolabs, Beverly, Mass.). “Hot start” PCR conditions coupled with a 60° C. annealing temperature were used to minimize primer-dimer formation. Amplified pool template DNA was desalted with a Centrisep column (Princeton Separations, Adelphia, N.J.) according to the manufacturer's recommended conditions, and used to transcribe the pool RNA for the next round of selection. The transcribed pool was gel purified on a 10% polyacrylamide gel every round. Table 6 shows the RNA concentration used per round of selection.

TABLE 6
RNA pool concentrations per round of selection.

| | rRmY | rGmH |
|---|---|---|
| Round | (pmoles pool used) | (pmoles pool used) |
| 1 | 200 | 200 |
| 2 | 110 | 40 |
| 3 | 65 | 100 |
| 4 | 50 | 170 |
| 5 | 80 | 100 |
| 6 | 100 | 110 |
| 7 | 50 | 70 |
| 8 | 120 | 60 |
| 9 | 120 | 80 |
| 10 | 130 | |
| 11 | 110 | |

The selection progress was monitored using the dot blot sandwich filter binding assay as described in Example 1A. The 5′-³²P-labeled pool RNA was refolded at 90° C. for 3 minutes and cooled to room temperature for 10 minutes. Next, pool RNA (trace concentration) was incubated with h-IL-23 DPBS plus 0.1 mg/mL tRNA for 30 minutes at room temperature and then applied to a nitrocellulose and nylon filter sandwich in a dot blot apparatus (Schleicher and Schuell). The percentage of pool RNA bound to the nitrocellulose was calculated and monitored approximately every 3 rounds with a single point screen (+/−250 nM h-IL-23). Pool K_{D} measurements were measured using a titration of h-IL-23 protein (R&D, Minneapolis, Minn.) and the dot blot apparatus as described above.

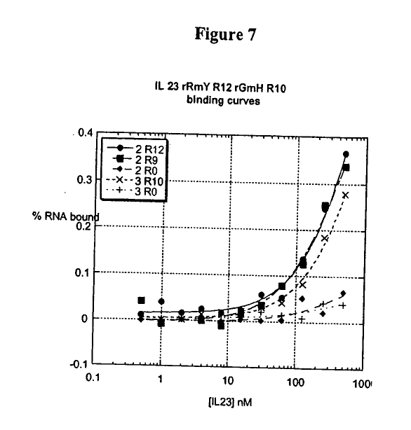

The rRmY h-IL-23 selection was enriched for h-IL-23 binding vs. the naïve pool after 4 rounds of selection (data not shown). The selection stringency was increased and the selection was continued for 8 more rounds. At Round 9 the pool K_{D} was approximately 500 nM or higher. The rGmH selection was enriched over the naïve pool binding at Round 10. The pool K_{D} was also approximately 500 nM or higher. FIG. 7 is a binding curve of rRmY and rGmH pool selection binding to h-IL-23. The pools were cloned using TOPO TA cloning kit (Invitrogen, Carlsbad, Calif.) and individual sequences were generated and tested for binding. A single point binding screen was initially performed on all crude rRmY clone transcriptions using a 1:200 dilution, +/−200 nM IL-23, plus 0.1 mg/mL competitor tRNA. A 10 point screen was then performed on 24 of the rRmY clones which showed the best binding in the single point screen. The 10 point screen was performed using zero to 480 nM IL-23 in 3 fold serial dilutions. Binding curves were generated (KaleidaGraph v. 3.51, Synergy Software) and KDS were estimated by fitting the data to the equation: fraction RNA bound=amplitude[h-IL-23]/K_{D}+[h-IL-23]). Table 7 below shows the sequence data for the rRmY selected aptamers that displayed binding affinity for h-IL-23. There was one group of 6 duplicate sequences and 4 pairs of 2 duplicate sequences out of the rRmY clones generated. Table 8 shows the binding characteristics of the rRmY clones thus tested. Clones were also tested from 48 crude rGmH clone transcriptions at a 1:200 dilution and 0.1 mg/mL tRNA was used as competitor. The average binding over background was only about 14%, whereas the average of the rRmY clones in the same assay was about 30%, with 10 clones higher than 40%. The sequences and binding characterization of the rGmH clones tested are not shown.

The nucleic acid sequences of the rRmY aptamers characterized in Table 7 are given below. The unique sequence of each aptamer in Table 7 begins at nucleotide 23, immediately following the sequence GGGAGAGGAGAGAACGUUCUAC (SEQ ID NO 69), and runs until it meets the 3′fixed nucleic acid sequence GCUGUCGAUCGAUCGAUCGAUG (SEQ ID NO 70).

Unless noted otherwise, individual sequences listed below are represented in the 5′ to 3′ orientation and represent the sequences of the aptamers that bind to IL-23 and/or IL-12 selected under rRmY SELEX™ conditions wherein the purines (A and G) are 2′-OH and the pyrimidines (U and C) are 2′-OMe. Each of the sequences listed in Table 7 may be derivatized with polyalkylene glycol (“PAG”) moieties and may or may not contain capping (e.g., a 3′-inverted dT).

TABLE 7
rRmY (Round 10) Sequences

SEQ ID NO 71
GGGAGAGGAGAGAACGUUCUACAAAUGAGAGCAGGCCGAAGAGGAGUCGC
UCGCUGUCGAUCGAUCGAUCGAUG
SEQ ID NO 72
GGGAGAGGAGAGAACGUUCUACAAAUGAGAGCAGGCCGAAAAGGAGUCGC
UCGCUGUCGAUCGAUCGAUCGAUG
SEQ ID NO 73
GGGAGAGGAGAGAACGUUCUACAAAUGAGAGCAGGCCGAAAAGGAGUCGC
UCGCUGUCGAUCGAUCGAUCGAUG
SEQ ID NO 74
GGGAGAGGAGAGAACGUUCUACGGUAAAGCAGGCUGACUGAAAGGUUGAA
GUCGCUGUCGAUCGAUCGAUCGAUG
SEQ ID NO 75
GGGAGAGGAGAGAACGUUCUACAGGUUAAGAGCAGGCUCAGGAAUGGAAG
UCGCUGUCGAUCGAUCGAUCGAUG
SEQ ID NO 76
GGGAGAGGAGAGAACGUUCUACAACAAAGCAGGCUCAUAGUAAUAUGGAA
GUCGCUGUCGAUCGAUCGAUCGAUG
SEQ ID NO 77
GGGAGAGGAGAGAACGUUCUACAACAAAGCAGGCUCAUAGUAAUAUGGAA
GUCGCUGUCGAUCGAUCGAUCGAUG
SEQ ID NO 78
GGGAGAGGAGAGAACGUUCUACAAAAGAGAGCAGGCCGAAAAGGAGUCGC
UCGCUGUCGAUCGAUCGAUCGAUG
SEQ ID NO 79
GGGAGAGGAGAGAACGUUCUACAAAAGGCAGGCUCAGGGGAUCACUGGAA
GUCGCUGUCGAUCGAUCGAUCGAUG
SEQ ID NO 80
GGGAGAGGAGAGAACGUUCUACAAGAUAUAAUUAAGGAUAAGUGCAAAGG
AGACGCUGUCGAUCGAUCGAUCGAUG
SEQ ID NO 81
GGGAGAGGAGAGAACGUUCUACGAAUGAGAGCAGGCCGAAAAGGAGUCGC
UCGCUGUCGAUCGAUCGAUCGAUG
SEQ ID NO 82
GGGAGAGGAGAGAACGUUCUACGAGAGGCAAGAGAGAGUCGCAUAAAAAA
GACGCUGUCGAUCGAUCGAUCGAUG
SEQ ID NO 83
GGGAGAGGAGAGAACGUUCUACGCAGGCUGUCGUAGACAAACGAUGAAGU
CGCGCUGUCGAUCGAUCGAUCGAUG
SEQ ID NO 84
GGGAGAGGAGAGAACGUUCUACGGAAAAAGAUAUGAAAGAAAGGAUUAAG
AGACGCUGUCGAUCGAUCGAUCGAUG
SEQ ID NO 85
GGGAGAGGAGAGAACGUUCUACGGAAGGNAACAANAGCACUGUUUGUGCA
GGCGCUGUCGAUCNAUCNAUCNAUG
SEQ ID NO 86
GGGAGAGGAGAGAACGUUCUACUAAUGCAGGUCAGUUACUACUGGAAGUC
GCUGUCGAUCGAUCGAUCGAUG
SEQ ID NO 87
AGGAGAGGAGAGAACGUUCUACUAGAAGCAGGCUCGAAUACAAUUCGGAA
GUCGCUGUCGAUCGAUCGAUCGAUG
SEQ ID NO 88
GGGAGAGGAGAGAACGUUCUACAUAAGCAGGCUCCGAUAGUAUUCGGGAA
GUCGCUGUCGAUCGAUCGAUCGAU

TABLE 8
rRmY IL-23 Clone Binding Data.

| SEQ | IL-23 K_{D} |
|---|---|
| ID No. | (nM) |
| 72 | 211.4 |
| 83 | 8.2 |
| 86 | 219.3 |
| 80 | 3786.3 |
| 75 | 479.4 |
| 74 | 257.0 |
| 81 | 303.2 |
| 77 | 258.9 |
| 73 | 101.4 |
| 88 | 101.2 |
| 84 | 602.5 |
| 78 | 123.7 |
| 76 | 77.2 |
| 87 | 122.3 |
| 71 | 124.0 |
| 85 | 239.9 |
| 82 | 198.6 |
| 79 | 806.7 |

**Assays performed in 1X DPBS (+Ca²⁺, Mg²⁺), 30 min RT incubation
**R&D IL-23 (carrier free protein)

### Example 1C

### Selections Against Human IL-23 with Deoxy/2′O-Methyl Nucleotide Containing Pools

An alternative selection was performed to obtain stabilized aptamers specific for IL-23 using deoxy purines (A and G) and 2′-O-Me pyrimidines (C and U) using the h-IL-23 strategy.

Pool Preparation. A DNA template with the sequence 5′-GGGAGAGGAGAGAACGTTCTACN₃₀CGCTGTCGATCGATCGATCGATG-3′ (ARC256, SEQ ID NO 3) was synthesized using an ABI EXPEDITE™ DNA synthesizer, and deprotected by standard methods. The series of N's in the DNA template (SEQ ID NO 3) can be any combination of nucleotides and gives rise to the unique sequence region of the resulting aptamers. The templates were amplified with the 5′ primer 5′-TAATACGACTCACTATAGGGAGAGGAGAGAACGTTCTAC-3′ (SEQ ID NO 67) and 3′ primer 5′-CATCGATCGATCGATCGACAGC-3′ (SEQ ID NO 89) and then used as a template for in vitro transcription with Y639F single mutant T7 RNA polymerase. Transcriptions were done at 37° C. overnight using 200 mM Hepes, 40 mM DTT, 2 mM spermidine, 0.01% Triton X-100, 10% PEG-8000, 9.6 mM MgCl₂, 2.9 mM MnCl₂, 2 mM NTPs, 2 mM GMP, 2 mM spermine, 0.01 units/μL inorganic pyrophosphatase, and 2 μg/mL Y639F single mutant T7 polymerase.

Selection: Each round of selection was initiated by immobilizing 20 pmoles of h-IL-23 to the surface of Nunc Maxisorp hydrophobic plates for 1 hour at room temperature in 100 μL of 1×PBS. The supernatant was then removed and the wells were washed 5 times with 120 μL wash buffer (1×PBS, 0.1 mg/mL tRNA and 0.1 mg/mL salmon sperm DNA (“ssDNA”)). In Round 1, a positive selection step was conducted: 100 pmoles of pool RNA (6×10¹³ unique molecules) were incubated in 100 μL binding buffer (1×PBS, 0.1 mg/mL tRNA and 0.1 mg/mL ssDNA) in the wells with immobilized protein target for 1 hour. The supernatant was then removed and the wells were washed 5 times with 120 μL wash buffer. In subsequent rounds a negative selection step was included. The pool RNA was also incubated for 1 hour at room temperature in empty wells to remove any plastic binding sequences from the pool before the positive selection step. Starting at Round 3, a second negative selection step was introduced. The target-immobilized wells were blocked for 1 hour at room temperature in 100 μL blocking buffer (1×PBS, 0.1 mg/mL tRNA, 0.1 mg/mL ssDNA and 0.1 mg/mL BSA) before the positive selection step. In all cases, the pool RNA bound to immobilized h-IL-23 was reverse transcribed directly in the selection plate after by the addition of RT mix (3′ primer, (SEQ ID NO 89)), and Thermoscript™ RT (Invitrogen, Carlsbad, Calif.), followed by incubation at 65° C. for 1 hour. The resulting cDNA was used as a template for PCR (Taq polymerase, New England Biolabs, Beverly, Mass.). “Hot start” PCR conditions coupled with a 68° C. annealing temperature were used to minimize primer-dimer formation. Amplified pool template DNA was desalted with a Micro Bio-Spin column (Bio-Rad, Hercules, Calif.) according to the manufacturer's recommended conditions and used to program transcription of the pool RNA for the next round of selection. The transcribed pool was gel purified on a 10% polyacrylamide gel every round.

Protein Binding Analysis. The selection progress was monitored using the sandwich filter binding assay previously described in Example 1A. The 5′-³²P-labeled pool RNA (trace concentration) was incubated with h-IL-23, 1×PBS plus 0.1 mg/mL tRNA, 0.1 mg/mL ssDNA and 0.1 mg/mL BSA for 30 minutes at room temperature and then applied to a nitrocellulose and nylon filter sandwich in a dot blot apparatus (Schleicher and Schuell, Keene, N.H.). The percentage of pool RNA bound to the nitrocellulose was calculated after Rounds 6, 7 and 8 with a seven point screen with h-IL-23 (0.25 nM, 0.5 nM, 1 nM, 4 nM, 16 nM, 64 nM and 128 nM). Pool K_{D} measurements were calculated as previously described.

The dRmY IL-23 selection was enriched for h-IL-23 binding vs. the naïve pool after 6 rounds of selection. At Round 8 the pool K_{D} was approximately 54 nM or higher. The Round 6, 7 and 8 pools were cloned using a TOPO TA cloning kit (Invitrogen, Carlsbad, Calif.) and individual sequences were generated. Table 9 lists the sequences of the dRmY clones generated from Round 6, 7 and 8 pools. Protein binding analysis was performed for each clone. Binding assays were performed in 1×PBS+0.1 mg/mL tRNA, 0.1 mg/mL salmon sperm DNA, 0.1 mg/mL BSA, for a 30 minute incubation at room temperature. Table 10 includes the binding characterization for these individual sequences.

The nucleic acid sequences of the dRmY aptamers characterized in Table 9 are given below. The unique sequence of each aptamer below begins at nucleotide 23, immediately following the sequence GGGAGAGGAGAGAACGUUCUAC (SEQ ID NO 69), and runs until it meets the 3′fixed nucleic acid sequence GCUGUCGAUCGAUCGAUCGAUG (SEQ ID NO 90).

Unless noted otherwise, individual sequences listed below are represented in the 5′ to 3′ orientation and represent the sequences of the aptamers that bind to IL-23 and/or IL-12 selected under dRmY SELEX™ conditions wherein the purines (A and G) are deoxy and the pyrimidines (U and C) are 2′-OMe. Each of the sequences listed in Table 9 may be derivatized with polyalkylene glycol (“PAG”) moieties and may or may not contain capping (e.g., a 3′-inverted dT).

TABLE 9
dRmY IL-23 clone sequences

SEQ ID NO 91 (ARC 489)
GGGAGAGGAGAGAACGUUCUACAGCGCCGGUGGGCGGGCAUUGGGUGGAU
GCGCUGUCGAUCGAUCGAUCGAUG
SEQ ID NO 92 (ARC 490)
GGGAGAGGAGAGAACGUUCUACAGCCUUUUGGGUAAGGGGAGGGGUGCCG
GUCGCUGUCGAUCGAUCGAUCGAUG
SEQ ID NO 93
GGGAGAGGAGAGAACGUUCUACGUAACGGGGUGGGAGGGGCGAACAACUU
GACGCUGUCGAUCGAUCGAUCGAUG
SEQ ID NO 94 (ARC 491)
GGGAGAGGAGAGAACGUUCUACAGCGCCGGUGGGUGGGCAUAGGGUGGAU
GCGCUGUCGAUCGAUCGAUCGAUG
SEQ ID NO 95
GGGAGAGGAGAGAACGUUCUACGGGCUACGGGGAUGGAGGGUGGGUCCCA
GACGCUGUCGAUCGAUCGAUCGAUG
SEQ ID NO 96
GGGAGAGGAGAGAACGUUCUACACGGGGUGGGAGGGGCGAGUCGCAUGGA
UGCGCUGUCGAUCGAUCGAUCGAUG
SEQ ID NO 97 (ARC492)
GGGAGAGGAGAGAACGUUCUACUCAAUGACCGCGCGAGGCUCUGGGAGAG
GGCGCUGUCGAUCGAUCGAUCGAUG

TABLE 10
dRmY IL-23 aptamer binding data

| SEQ | | IL-12 K_{D} |
|---|---|---|
| ID No. | IL-23 K_{D} (nM) | (nM) |
| 91 | 4.0 | 17.2 |
| 92 | 26.0 | 37.1 |
| 93 | 186.2 | Not tested |
| 94 | 17.1 | 93.0 |
| 95 | 432.6 | Not tested |
| 96 | 209.7 | Not tested |
| 97 | NB | NB |

**Assays performed in 1X PBS + 0.1 mg/mL tRNA, 0.1 mg/mL ssDNA, 0.1 mg/mL BSA, 30 min RT incubation
**R&D IL-23 (carrier free protein)
N.B. = no binding detectable

### Example 1D

### Additional Selections Against Human IL-23 with Deoxy/2′O-Methyl Nucleotide Containing Pools

Introduction: Three selections strategies were used to identify aptamers to h-IL-23 using a pool containing deoxy/2′O-Methyl nucleotides. These selections used 2′O-Me C, and U and deoxy A and G. The first selection strategy (dRmY h-IL-23) was a direct selection against h-IL-23. In the second selection strategy (dRmY h-IL-23/IL-12neg), h-IL-12 was included in the negative selection step to drive enrichment of aptamers binding to p19, the subdomain unique to h-IL-23. In the third selection strategy (dRmY h-IL-23-S), increased stringency was used in the positive selection by including long washes to drive the selection to select for higher affinity aptamers. All three selection strategies yielded aptamers to h-IL-23. Several aptamers are specific for h-IL-23, and several show cross reactivity between h-IL-23 and h-IL-12.

dRmY Selection: Round 1 of the dRmY h-IL-23 selection began with 3×10¹⁴ molecules of a 2′O-Me C, and U and deoxy A and G modified RNA pool with the sequence 5′-GGGAGAGGAGAGAACGUUCUAC-N30-GGUCGAUCGAUCGAUCAUCGAUG-3′ (ARC520) (SEQ ID NO 98), which was synthesized using an ABI EXPEDITE™ DNA synthesizer, and deprotected by standard methods. The series of N's in the template (SEQ ID NO 98) can be any combination of nucleotides and gives rise to the unique sequence region of the resulting aptamers.

Each round of selection was initiated by immobilizing 20 pmoles of h-IL-23 to the surface of Nunc Maxisorp hydrophobic plates for 1 hour at room temperature in 100 μL of 1×PBS. The supernatant was then removed and the wells were washed 5 times with 120 μL wash buffer (1×PBS, 0.1 mg/mL tRNA and 0.1 mg/mL salmon sperm DNA (“ssDNA”)). In Round 1, 500 pmoles of pool RNA (3×10¹⁴ molecules) were incubated in 100 μL binding buffer (1×PBS, 0.1 mg/mL tRNA and 0.1 mg/mL ssDNA) in the well with immobilized protein target for 1 hour. The supernatant was then removed and the well was washed 5 times with 120 μL wash buffer. In subsequent rounds a negative selection step was included in which pool RNA was also incubated for 1 hour at room temperature in an empty well to remove any plastic binding sequences from the pool before the positive selection step.

Starting at Round 3, a second negative selection step was introduced. The pool was subjected to a 1 hour incubation in target-immobilized wells that were blocked for 1 hour at room temperature with 100 μL blocking buffer (1×PBS, 0.1 mg/mL tRNA, 0.1 mg/mL ssDNA and 0.1 mg/mL BSA) before the positive selection step (Table 1 lA). At Round 3, the dRmY h-IL-23 pool was split into the dRmY h-IL-23/IL-12neg selection by subjecting the pool to an additional 1 hour negative incubation step at room temperature in a well that had been blocked for 1 hour at room temperature with 20 pmoles of h-IL-12 and washed 5 times with 120 μL wash buffer, which occurred prior to the positive h-IL-23 positive incubation. The pool was split into additional h-IL-12 blocked wells in later rounds to increase the stringency (See Table 11B).

An additional method to increase discrimination between h-IL-23 and h-IL-12 binding was to add h-IL-12 to the positive selection along with the pool at a low concentration, in which the specific h-IL-23 binders would bind to the immobilized h-IL-23, and the h-IL-12 binders would be washed away after the 1 hour incubation. The dRmY h-IL-23-S selection was split from the dRmY h-IL-23 pool at Round 6 with the addition of “stringent washes” in the positive selection, in which after the 1 hour incubation with h-IL-23, the pool was removed, then 100 μL of 1×PBS, 0.1 mg/mL tRNA, and 0.1 mg/mL ssDNA was added and incubated for 30 minutes (Table 11 C). This stringent wash procedure was removed and repeated, with the intentions of selecting for molecules with high affinities.

In all cases, the pool RNA bound to immobilized h-IL-23 was reverse transcribed directly in the selection plate by the addition of RT mix (3′ primer, 5′-CATCGATGATCGATCGATCGAC-3′ (SEQ ID NO 100)), and Thermoscript™ RT, (Invitrogen, Carlsbad, Calif.) followed by incubation at 65° C. for 1 hour. The resulting cDNA was used as a template for PCR (20 mM Tris pH 8.4, 50 mM KC1, 2 mM MgCl₂, 0.5 μM of 5′ primer 5′-TAATACGACTCACTATAGGGAGAGGAGAGAACGTTCTAC-3′ (SEQ ID NO 99), 0.5 μM of 3′ primer (SEQ ID NO 100), 0.5 mM each dNTP, 0.05 units/μL Taq polymerase (New England Biolabs, Beverly, Mass.)). PCR reactions were done under the following cycling conditions: a): 94° C. for 30 seconds; b) 55° C. for 30 seconds; c) 72° C. for 30 seconds. The cycles were repeated until sufficient PCR product was generated. The minimum number of cycles required to generate sufficient PCR product is reported in Tables 11A-11C as the “PCR Threshold”.

The PCR templates were purified using the QIAquick PCR purification kit (Qiagen, Valencia, Calif.) and used to program transcription of the pool RNA for the next round of selection. Templates were transcribed overnight at 37° C. using 200 mM Hepes, 40 mM DTT, 2 mM spermidine, 0.01% Triton X-100, 10% PEG-8000, 9.6 mM MgCl₂, 2.9 mM MnCl₂, 2 mM NTPs, 2 mM GMP, 2 mM spermine, 0.01 units/mL inorganic pyrophosphatase, and 2 μg/mL Y639F single mutant T7 polymerase. Transcription reactions were quenched with 50 mM EDTA and ethanol precipitated, then purified on a 1.5 mm denaturing polyacrylamide gel (8 M urea, 10% acrylamide; 19:1 acrylamide:bisacrylamide). Pool RNA was removed from the gel by passive elution at 37° C. in 300 mM NaOAc, 20 mM EDTA, followed by ethanol precipitation. The selection conditions for each round are provided in the following tables.

TABLE 11A
dRmY hIL-23 selection conditions
IL-23

| | RNA | | | BSA- | |
|---|---|---|---|---|---|
| | pool | IL-23 | | blocked | |
| | conc | conc | untreated | well | PCR |
| Round # | (μM) | (μM) | well neg | neg | Threshold |
| 1 | 5 | 0.2 | none | none | 18 |
| 2 | 0.6 | 0.2 | 1 hr | none | 17 |
| 3 | 0.75 | 0.2 | 1 hr | 1 hr | 17 |
| 4 | 1 | 0.2 | 1 hr | 1 hr | 17 |
| 5 | 0.75 | 0.2 | 1 hr | 1 hr | 17 |
| 6 | 1 | 0.2 | 1 hr | 1 hr | 15 |
| 7 | 1 | 0.2 | 1 hr | 1 hr | 15 |
| 8 | 1 | 0.2 | 1 hr | 1 hr | 16 |

TABLE 11B
dRmY IL-23/IL-12neg selection conditions
IL-23/12neg

| | RNA | | un- | BSA- | IL-12 | | IL-12 | |
|---|---|---|---|---|---|---|---|---|
| | pool | IL-23 | treated | blocked | neg | # IL- | pos | PCR |
| Round | conc | conc | well | well | conc | 12 | conc | Thresh- |
| # | (μM) | (μM) | neg | neg | (μM) | wells | (μM) | old |
| 1 | 5 | 0.2 | none | none | 0 | 0 | 0 | 18 |
| 2 | 0.6 | 0.2 | 1 hr | none | 0 | 0 | 0 | 17 |
| 3 | 0.75 | 0.2 | 1 hr | 1 hr | 0.2 | 1 | 0 | 17 |
| 4 | 1 | 0.2 | 1 hr | 1 hr | 0.2 | 1 | 0 | 17 |
| 5 | 0.75 | 0.2 | 1 hr | 1 hr | 0.2 | 2 | 0 | 17 |
| 6 | 1 | 0.2 | 1 hr | 1 hr | 0.2 | 2 | 0 | 15 |
| 7 | 1 | 0.2 | 1 hr | 1 hr | 0.2 | 3 | 0.02 | 15 |
| 8 | 1 | 0.2 | 1 hr | 1 hr | 0.2 | 3 | 0.05 | 15 |

TABLE 11C
dRmY hIL-23-S selection conditions
IL-23S

| | RNA | | | BSA- | # | |
|---|---|---|---|---|---|---|
| | pool | IL-23 | | blocked | 30 min | |
| | conc | conc | untreated | well | positive | PCR |
| Round # | (μM) | (μM) | well neg | neg | washes | Threshold |
| 1 | 5 | 0.2 | none | none | 0 | 18 |
| 2 | 0.6 | 0.2 | 1 hr | none | 0 | 17 |
| 3 | 0.75 | 0.2 | 1 hr | 1 hr | 0 | 17 |
| 4 | 1 | 0.2 | 1 hr | 1 hr | 0 | 17 |
| 5 | 0.75 | 0.2 | 1 hr | 1 hr | 0 | 17 |
| 6 | 1 | 0.2 | 1 hr | 1 hr | 2 | 15 |
| 7 | 1 | 0.2 | 1 hr | 1 hr | 2 | 16 |
| 8 | 1 | 0.2 | 1 hr | 1 hr | 2 | 16 |

Protein Binding Analysis: Dot blot binding assays were performed throughout the selections to monitor the protein binding affinity of the pools as previously described in Example 1A. When a significant positive ratio of binding of RNA in the presence of h-IL-23 versus in the absence of h-IL-23 was seen, the pools were cloned using a TOPO TA cloning kit (Invitrogen, Carlsbad, Calif.) according to the manufacturer's instructions. Similar sequences were seen in all three selections from the pools having gone through six rounds, and 45 unique clones amongst the three selections were chosen for screening. The 45 clones were synthesized on an ABI EXPEDITE™ DNA synthesizer, then deprotected by standard methods. The 45 individual clones were gel purified on a 10% PAGE gel, and the RNA was passively eluted in 300 mM NaOAc and 20 mM EDTA, followed by ethanol precipitation.

The clones were 5′ end labeled with γ-³²P ATP, and were assayed for both IL-23 and IL-12 binding in a 3-point dot blot screen (0 nM, 20 nM, and 100 nM h-IL-23; 0 nM, 20 nM, and 100 nM h-IL-12) (data not shown). Clones showing significant binding in the 20 nM and 100 nM protein conditions for both IL-23 and IL-12 were further assayed for K_{D} determination using a protein titration from 0 nM to 480 nM (3 fold dilutions) in the dot blot assay previously described. K_{D} values were determined by fitting an equation describing a 1:1 RNA:protein complex to the resulting data (fraction aptamer bound=amplitude*([IL-23]/(K_{D}+[IL-23]))+background binding) (KaleidaGraph v. 3.51, Synergy Software). Results of protein binding characterization for the higher affinity clones are tabulated in Table 13, and corresponding clone sequences are listed in Table 12.

The nucleic acid sequences of the dRmY aptamers characterized in Table 12 are given below. The unique sequence of each aptamer below begins at nucleotide 23, immediately following the sequence GGGAGAGGAGAGAACGUUCUAC (SEQ ID NO 101), and runs until it meets the 3′fixed nucleic acid sequence GUCGAUCGAUCGAUCAUCGAUG (SEQ ID NO 102).

Unless noted otherwise, individual sequences listed below are represented in the 5′ to 3′ orientation and represent the sequences of the aptamers that bind to IL-23 and/or IL-12 selected under dRmY SELEX™ conditions wherein the purines (A and G) are deoxy and the pyrimidines (C and U) are 2′-OMe. Each of the sequences listed in Table 12 may be derivatized with polyalkylene glycol (“PAG”) moieties and may or may not contain capping (e.g., a 3′-inverted dT).

TABLE 12
dRmY clone sequences

SEQ ID NO 103 (ARC611)
GGGAGAGGAGAGAACGUUCUACAGGCAAGGCAAUUGGGGAGUGUGGGUGG
GGGGUCGAUCGAUCGAUCAUCGAUG
SEQ ID NO 104 (ARC612)
GGGAGAGGAGAGAACGUUCUACAGGCAAGUAAUUGGGGAGUGCGGGCGGG
GGGGUCGAUCGAUCGAUCAUCGAUG
SEQ ID NO 105 (ARC614)
GGGAGAGGAGAGAACGUUCUACAAGGCGGUACGGGGAGUGUGGGUUGGGG
CCGGUCGAUCGAUCGAUCAUCGAUG
SEQ ID NO 106 (ARC616)
GGGAGAGGAGAGAACGUUCUACGAUAUAGGCGGUACGGGGGGAGUGGGCU
GGGGUCGAUCGAUCGAUCAUCGAUG
SEQ ID NO 107 (ARC620)
GGGAGAGGAGAGAACGUUCUACAGGAAAGGCGCUUGCGGGGGGUGAGGGA
GGGGUCGAUCGAUCGAUCAUCGAUG
SEQ ID NO 108 (ARC621)
GGGAGAGGAGAGAACGUUCUACAGGCGGUUACGGGGGAUGCGGGUGGGAC
AGGUCGAUCGAUCGAUCAUCGAUG
SEQ ID NO 109 (ARC626)
GGGAGAGGAGAGAACGUUCUACAGGCAAGUAAUUGGGGAGUGCGGGCGGG
GGGUCGAUCGAUCGAUCAUCGAUG
SEQ ID NO 110 (ARC627)
GGGAGAGGAGAGAACGUUCUACAGGCAAGUAAUUGGGGAGUGCGGGCGGG
GUGUCGAUCGAUCGAUCAUCGAUG
SEQ ID NO 111 (ARC628)
GGGAGAGGAGAGAACGUUCUACAGGCAGGCAAUUGGGGAGCGUGGGUGGG
GGGUCGAUCGAUCGAUCAUCGAUG
SEQ ID NO 112 (ARC632)
GGGAGAGGAGAGAACGUUCUACAAUUGCAGGUGGUGCCGGGGGUUGGGGC
GGGUCGAUCGAUCGAUCAUCGAUG
SEQ ID NO 113 (ARC635)
GGGAGAGGAGAGAACGUUCUACAGGCUCAAAAGAGGGGGAUGUGGGAGGG
GGUCGAUCGAUCGAUCAUCGAUG
SEQ ID NO 114 (ARC642)
GGGAGAGGAGAGAACGUUCUACAGGCGCAGCCAGCGGGGAGUGAGGGUGG
GGGUCGAUCGAUCGAUCAUCGAUG
SEQ ID NO 115 (ARC643)
GGGAGAGGAGAGAACGUUCUACAGGCCGAUGAGGGGGAGCAGUGGGUGGG
GGGUCGAUCGAUCGAUCAUCGAUG
SEQ ID NO 116 ARC644)
GGGAGAGGAGAGAACGUUCUACUAGUGAGGCGGUAACGGGGGGUGAGGGU
GGGGUCGAUCGAUCGAUCAUCGAUG
SEQ ID NO 117 (ARC645)
GGGAGAGGAGAGAACGUUCUACAGGUAGGCAAGAUAUUGGGGGAAGCGGG
UGGGGUCGAUCGAUCGAUCAUCGAUG
SEQ ID NO 118 (ARC 646)
GGGAGAGGAGAGAACGUUCUACACAUGGCUCGAAAGAGGGGCGUGAGGGU
GGGGUCGAUCGAUCGAUCAUCGAUG

TABLE 13
Summary of dRmY clone binding

| SEQ | | | K_{D} hIL- | K_{D} hIL- |
|---|---|---|---|---|
| ID NO | ARC # | Selection | 23 (nM) | 12 (nM) |
| 103 | ARC611 | R7 hIL-23/12neg | 21.3 | 123.1 |
| 104 | ARC612 | R7 hIL-23/12neg | 5.8 | 41.7 |
| 105 | ARC614 | R7 hIL-23/12neg | 3.1 | 54.4 |
| 106 | ARC616 | R7 hIL-23/12neg | 13.1 | 52.1 |
| 107 | ARC620 | R7 hIL-23/12neg | 44.8 | 178.7 |
| 108 | ARC621 | R7 hIL-23/12neg | 28.8 | 111.9 |
| 109 | ARC626 | R7 hIL-23S | 10.1 | 69.8 |
| 110 | ARC627 | R7 hIL-23S | 7 | 79.5 |
| 111 | ARC628 | R7 hIL-23S | 57.8 | 146.5 |
| 112 | ARC632 | R7 hIL-23S | 19.1 | 63.9 |
| 113 | ARC635 | R7 hIL-23S | 171.5 | 430.9 |
| 114 | ARC642 | R7 hIL-23 | 37.2 | 188.3 |
| 115 | ARC643 | R7 hIL-23S | 71.6 | 309.4 |
| 116 | ARC644 | R7 hIL-23 | 34.5 | 192.9 |
| 117 | ARC645 | R7 hIL-23 | 33.5 | 137.3 |
| 118 | ARC646 | R7 hIL-23 | 207.9 | 382.6 |

*30 min RT incubation for K_{D} determination in dot blot assay
*1X PBS + 0.1 mg/mL tRNA, salmon sperm DNA, BSA reaction buffer

### Human IL-23 Aptamer Selections Summary

The different selection conditions and strategies for IL-23 SELEX™ yielded several aptamers, stabilized and/or minimized, having different binding characteristics. The rRfY selected aptamers have affinities approximately in the 15 nM to 460 nM range, and prior to any post-SELEX™ optimization, have cellular potency with IC₅₀s approximately in the 50 nM-to 5 μM range. These can be further minimized with appropriate gains in binding characteristics and are expected to show increased potency in cell based assays. These aptamers also show the greatest distinction between IL-23, having a greater than hundred fold discrimination of IL-23 to IL-12.

The aptamers obtained under the rRmY selection conditions have affinities ranging from approximately 8 nM to 3 μM. However, their cellular potency is lower than the rRfY aptamers' potency. As for the rGmH constructs a single point screen was done, but not carried any further because their extent of binding over background was not as good as the rRmY clones. 48 crude rGmH clone transcriptions were used at a 1:200 dilution and 0.1 mg/mL tRNA was used as competitor. The average binding over background was only about 14%, whereas the rRmY clone's average in the same assay was about 30%, with 10 clones higher than 40%.

The dRmY selected aptamers have high affinities in the range of 3 nM to 200 nM, and prior to any post-SELEX™ optimization, show a remarkable cellular potency with IC₅₀s in the range of ˜50 nM to ˜500 nM (described in Example 3 below). Some of these aptamers also have a distinction of approximately 4 fold for IL-23 to IL-12, which may be improved upon by further optimization.

### Example 1E

### Selections Against Mouse (“m”)—IL-23 with 2′-F Pyrimidine Containing Pools (rRfY)

Introduction: Two selections strategies were used to identify aptamers to mIL-23 using a pool consisting of 2′-OH purine and 2′-F pyrimidine nucleotides (rRfY composition). The first selection strategy (mIL-23) was a direct selection against mIL-23. The second selection strategy (mIL-23S) was a more stringent selection, in which the initial rounds had lower concentrations of RNA and protein in an attempt to drive the selection towards higher affinity binders. Both selection strategies yielded aptamers to mIL-23.

Selection: Two selections (mIL-23 and mIL-23S) began with incubation of 2×10¹⁴ molecules of 2° F. pyrimidine modified pool with the sequence 5′ GGAGCGCACUCAGCCAC-N40-UUUCGACCUCUCUGCUAGC 3′ (ARC275) (SEQ ID NO 119), including a spike of γ³²P ATP 5′ end labeled pool, with mouse IL-23 (isolated in-house). The series of N's in the template (SEQ ID NO 119) can be any combination of nucleotides and gives rise to the unique sequence region of the resulting aptamers.

In Round 1 of the mIL-23 selection, pool RNA was incubated with 50 pmoles of protein in a final volume of 100 μL for 1 hr at room temperature. In Round 1 of the mIL-23S selection, pool RNA was incubated with 65 pmoles of mIL-23 in a final volume of 1300 μL for 1 hr at room temperature. Selections were performed in 1×PBS buffer. RNA:mIL-23 complexes and free RNA molecules were separated using 0.45 μm nitrocellulose spin columns from Schleicher & Schuell (Keene, N.H.). The columns were pre-washed with 1 mL 1×PBS, and then the RNA:protein containing solutions were added to the columns and spun in a centrifuge at 2000 rpm for 1 minute. Buffer washes were performed to remove nonspecific binders from the filters (Round 1, 2×500 μL 1×PBS; in later rounds, more stringent washes of increased number and volume to enrich for specific binders), then the RNA:protein complexes attached to the filters were eluted with 2×200 μL washes (2×100 μL washes in later rounds) of elution buffer (7 M urea, 100 mM sodium acetate, 3 mM EDTA, pre-heated to 90° C.). The eluted RNA was precipitated (40 μg glycogen, 1 volume isopropanol). The RNA was reverse transcribed with the Thermoscript™RT-PCR system (Invitrogen, Carlsbad, Calif.) according to the manufacturer's instructions, using the 3′ primer 5′GCTAGCAGAGAGGTCGAAA 3′ (SEQ ID NO 121), followed by PCR amplification (20 mM Tris pH 8.4, 50 mM KCl, 2 mM MgCl₂, 0.5 μM of 5′ primer 5′TAATACGACTCACTATAGGAGCGCACTCAGCCAC 3′ (SEQ ID NO 120), 0.5 HM of 3′ primer (SEQ ID 121), 0.5 mM each dNTP, 0.05 units/μL Taq polymerase (New England Biolabs, Beverly, Mass.)). PCR reactions were done under the following cycling conditions: a) 94° C. for 30 seconds; b) 60° C. for 30 seconds; c) 72° C. for 30 seconds. The cycles were repeated until sufficient PCR product was generated. The minimum number of cycles required to generate sufficient PCR product is reported in Table 14 as the “PCR Threshold”.

The PCR templates were purified using the QIAquick PCR purification kit (Qiagen, Valencia, Calif.). Templates were transcribed using α³²P GTP body labeling overnight at 37° C. (4% PEG-8000, 40 mM Tris pH 8.0, 12 mM MgCl₂, 1 mM spermidine, 0.002% Triton X-100, 3 mM 2′OH purines, 3 mM 2° F. pyrimidines, 25 mM DTT, 0.25 units/100 μL inorganic pyrophosphatase, 2 μg/mL T7 Y639F single mutant RNA polymerase, 5 uCi α³²P GTP).

Subsequent rounds were repeated using the same method as for Round 1, but with the addition of a negative selection step. Prior to incubation with protein target, the pool RNA was passed through a 0.45 micron nitrocellulose filter column to remove filter binding sequences, then the filtrate was carried on into the positive selection step. In alternating rounds the pool RNA was gel purified. Transcription reactions were quenched with 50 mM EDTA and ethanol precipitated then purified on a 1.5 mm denaturing polyacrylamide gels (8 M urea, 10% acrylamide; 19:1 acrylamide:bisacrylamide). Pool RNA was removed from the gel by passive elution in 300 mM NaOAc, 20 mM EDTA, followed by ethanol precipitation with the addition of 300 mM sodium acetate and 2.5 volumes of ethanol.

The RNA remained in excess of the protein throughout the selections (˜1 μM RNA). The protein concentration was dropped to varying lower concentrations based on the particular selection. Competitor tRNA was added to the binding reactions at 0.1 mg/mL starting at Round 2 or 3, depending on the selection. A total of 7 rounds were completed, with binding assays performed at select rounds. Table 14 contains the selection details including pool RNA concentration, protein concentration, and tRNA concentration used for each round. Elution values (ratio of CPM values of protein-bound RNA versus total RNA flowing through the filter column) along with binding assays were used to monitor selection progress.

TABLE 14
rRfY mIL-23 Selection conditions:

| | | | | | | |
|---|---|---|---|---|---|---|
| | RNA | | | | | |
| | pool | protein | | | | |
| | conc | conc | | tRNA | | PCR |
| Round # | (μM) | (nM) | neg | (mg/mL) | % elution | Threshold |

1. rRfY mIL-23

| | | | | | | |
|---|---|---|---|---|---|---|
| 1 | 3.3 | 500 | none | 0 | 2.64 | 8 |
| 2 | 1 | 500 | filter | 0.1 | 4.24 | 8 |
| 3 | ~1 | 200 | filter | 0.1 | 0.73 | 10 |
| 4 | 1 | 200 | filter | 0.1 | 3.71 | 8 |
| 5 | ~1 | 100 | filter | 0.1 | 0.41 | 10 |
| 6 | 1 | 100 | filter | 0.1 | 9.27 | 8 |
| 7 | ~1 | 100 | filter | 0.1 | 0.87 | 9 |

2. rRfY mIL-23S (stringent)

| | | | | | | |
|---|---|---|---|---|---|---|
| 1 | 0.25 | 50 | none | 0 | 2.79 | 8 |
| 2 | 0.1 | 50 | filter | 0 | 4.14 | 8 |
| 3 | ~1 | 50 | filter | 0.1 | 0.16 | 11 |
| 4 | 1 | 50 | filter | 0.1 | 2.57 | 8 |
| 5 | ~1 | 25 | filter | 0.1 | 0.42 | 10 |
| 6 | 0.8 | 25 | filter | 0.1 | 10.29 | 8 |
| 7 | ~1 | 25 | filter | 0.1 | 0.13 | 10 |

rRfY mIL-23 Protein Binding Analysis: Dot blot binding assays were performed throughout the selections to monitor the protein binding affinity of the pools as previously described. When a significant level of binding of RNA in the presence of mIL-23 was observed, the pools were cloned using a TOPO TA cloning kit (Invitrogen, Carlsbad, Calif.) according to the manufacturer's instructions. For both mIL-23 selections, the Round 7 pool templates were cloned, and 16 individual clones from each selection were assayed using an 8-point mIL-23 titration. Seven of the 32 total clones screened had specific binding curves and are listed below in Table 16. Table 15 lists the corresponding sequences. All others displayed nonspecific binding curves similar to the unselected naïve pool. Clones with high affinity to mIL-23 were subsequently screened for protein binding against mouse IL-12, human IL-23 and human IL-12 in the same manner.

The nucleic acid sequences of the rRfY aptamers characterized in Table 15 are given below. The unique sequence of each aptamer below begins at nucleotide 18, immediately following the sequence GGAGCGCACUCAGCCAC (SEQ ID NO 122), and runs until it meets the 3′fixed nucleic acid sequence UUUCGACCUCUCUGCUAGC (SEQ ID NO 123).

Unless noted otherwise, individual sequences listed below are represented in the 5′ to 3′ orientation and represent the sequences that bind to mouse IL-23 selected under rRfY SELEX™ conditions wherein the purines (A and G) are 2′-OH and the pyrimidines (C and U) are 2′-fluoro. Each of the sequences listed in Table 15 may be derivatized with polyalkylene glycol (“PAG”) moieties and may or may not contain capping (e.g., a 3′-inverted dT).

TABLE 15
mIL-23 rRfY Clone Sequences

SEQ ID NO 124 (ARC1628)
GGAGCGCACUCAGCCACAGGUGGCUUAAUACUGUAAAGACGUGCGCGCAG
AGGGAUUUUCGACCUCUCUGCUAGC
SEQ ID NO 125 (ARC1629)
GGAGCGCACUCAGCCACCGUAAUUCACAAGGUCCCUGAGUGCAGGGUUGU
AUGUUUGUUUCGACCUCUCUGCUAGC
SEQ ID NO 126 (ARC1630)
GGAGCGCACUCAGCCACUCUACUCGAUAUAGUUUAUCGAGCCGGUGGUAG
AUUAUGAUUUCGACCUCUCUGCUAGC
SEQ ID NO 127 (ARC1631)
GGAGCGCACUCAGCCACGCCUACAAUUCACUGUGAUAUAUCGAAUUAUAG
CCCUGGUUUCGACCUGUCUGCUAGC
SEQ ID NO 128 (ARC1632)
GGAGCGCACUCAGCCACCGGCUUAAUAUCCAAUAGGAACGUUCGCUCUGA
GCAGGCGUUUCGACCUCUCUGCUAGC
SEQ ID NO 129 (ARC1633)
GGAGCGCACUCAGCCACAGCUCGGUGGCUUAAUAUCUAUGUGAACGUGCG
CAACAGCUUUCGACCUCUCUGCUAGC
SEQ ID NO 130 (ARC1634)
GGAGCGCACUCAGCCACCUUGGGCUUAAUACCUAUCGGAUGUGCGCCUAG
CACGGAAUUUCGACCUCUCUGCUAGC

TABLE 16
mIL-23 rRfY Clone binding activity

| SEQ ID | | | K_{D} mIL-23 | K_{D} mIL-12 | K_{D} hIL-23 | K_{D} hIL- |
|---|---|---|---|---|---|---|
| NO | Clone Name | Selection | (nM) | (nM) | (nM) | 12 (nM) |
| 124 | ARC1628 | R7 mIL-23 | 2 | 6 | 52 | 161 |
| 125 | ARC1629 | R7 mIL-23 | 34 | 103 | 31 | 75 |
| 126 | ARC1630 | R7 mIL-23S | 14 | 18 | 65 | 239 |
| 127 | ARC1631 | R7 mIL-23S | 33 | 72 | 39 | 69 |
| 128 | ARC1632 | R7 mIL-23S | 13 | 16 | 91 | 186 |
| 129 | ARC1633 | R7 mIL-23S | 17 | 44 | 79 | 195 |
| 130 | ARC1634 | R7 mIL-23S | 3 | 29 | 39 | 63 |

*30 min RT incubation for K_{D} determination
*1X PBS + 0.1 mg/mL BSA reaction buffer

### Example 1F

### Selections for Mouse IL-23 Aptamers with Specificity Against Mouse IL-12

Introduction. One selection was performed to identify aptamers to mouse-IL-23 (mIL-23) with specificity against mouse IL-12 (mIL-12). This selection was split off from the rRfY selection mIL-23S described in the above section starting at Round 3. This selection yielded aptamers to mIL-23 that had ˜3-5-fold specificity over mIL-12.

mIL-23S/mL-12 neg rRfY Selection. To obtain mouse IL-23 aptamers with specificity against mouse IL-12, mouse IL-12 was included in a negative selection, similar to the protein in negative (PN-IL-23) selection described above in Example 1A. The resultant RNA from Round 2 of the mIL-23S selection described in Example 1E above was used to start the R3PN mIL-23/12neg selection, in which mIL-12 was included in the negative step of selection. Nine rounds of selection were performed, with binding assays performed at select rounds. Table 17 summarizes the selection conditions including pool RNA concentration, protein concentration, and tRNA concentration used for each round. Elution values (ratio of CPM values of protein-bound RNA versus total RNA flowing through the filter column) along with binding assays were used to monitor selection progress.

TABLE 17
rRfY mIL-23S/mIL-12 neg Filter Selection Summary

| | RNA | | | | neg | | |
|---|---|---|---|---|---|---|---|
| | pool | protein | | tRNA | mIL12 | | |
| Round | conc | conc | | (mg/ | conc | % | PCR |
| # | (μM) | (nM) | neg | mL) | (nM) | elution | cycle # |
| 1 | 0.25 | 50 | none | 0 | 0 | 2.79 | 8 |
| 2 | 0.1 | 50 | filter | 0 | 0 | 4.14 | 8 |
| 3 | ~1 | 500 | filter/IL12 | 0.1 | 250 | 1.33 | 10 |
| 4 | 1 | 500 | filter/IL12 | 0.1 | 500 | 1.68 | 8 |
| 5 | 1 | 250 | filter/IL12 | 0.1 | 250 | 0.89 | 9 |
| 6 | 1 | 200 | filter/IL12 | 0.1 | 200 | 1.47 | 8 |
| 7 | 1 | 150 | filter/IL12 | 0.1 | 150 | 1.39 | 8 |
| 8 | 1 | 150 | filter/IL12 | 0.1 | 150 | 3.73 | 8 |
| 9 | 1 | 150 | filter/IL12 | 0.1 | 150 | 2.98 | 8 |

Selection buffer: 1X PBS
*1 hr positive incubation

rRfY mIL-23S/mL-12 neg Protein Binding Analysis. The dot blot binding assays previously described were performed throughout the selection to monitor the protein binding affinity of the pool. Trace ³²P-labeled RNA was combined with mIL-23 or mIL-12 and incubated at room temperature for 30 min in 1×PBS plus 0.1 mg/mL BSA for a final volume of 30 μL. The reaction was added to a dot blot apparatus (Schleicher and Schuell Minifold-1 Dot Blot, Acrylic). Binding curves were generated as described in previous sections. When a significant level of binding of RNA in the presence of mIL-23 was observed, the pool was cloned using the TOPO TA cloning kit (Invitrogen, Carlsbad, Calif.) according to the manufacturer's instructions. The Round 9 pool template was cloned, and 10 individual clones from the selection were assayed in an 8-point dot blot titration against mIL-23. Clones that bound significantly to mIL-23 were then screened for binding to mIL-12. Table 18 summarizes protein binding characterization of the binding clones. Four of the 10 total clones screened bound specifically to mIL-23 and mIL-12 at varying affinities. All other clones displayed nonspecific binding curves similar to the unselected naïve pool. The sequences for the four binding clones are listed in Table 19 below.

TABLE 18
rRfY mIL-23S/mIL-12 neg Clone binding activity

| | | K_{D} mIL-23 | K_{D} mIL-12 |
|---|---|---|---|
| SEQ ID NO | Clone Name | (nM) | (nM) |
| 131 | AMX369.F1 | 63 | 165 |
| 132 | AMX369.H1 | 23 | 194 |
| 133 | AMX369.B2 | 49 | 252 |
| 134 | AMX369.G3 | 106 | 261 |

*30 min RT incubation for K_{D} determination
*1X PBS + 0.1 mg/mL BSA reaction buffer

The nucleic acid sequences of the rRfY aptamers characterized in Table 19 are given below. The unique sequence of each aptamer below begins at nucleotide 18, immediately following the sequence GGAGCGCACUCAGCCAC (SEQ ID NO 122), and runs until it meets the 3′fixed nucleic acid sequence UUUCGACCUCUCUGCUAGC (SEQ ID NO 123).

Unless noted otherwise, individual sequences listed below are represented in the 5′ to 3′ orientation and represent the sequences that bind to mouse IL-23 selected under rRfY SELEX™ conditions wherein the purines (A and G) are 2′-OH and the pyrimidines (U and C) are 2′-fluoro. Each of the sequences listed in Table 19 may be derivatized with polyalkylene glycol (“PAG”) moieties and may or may not contain capping (e.g., a 3′-inverted dT).

TABLE 19
rRfY mIL-235/mIL-12 neg Sequence Information

SEQ ID NO 131 (AMX(369)_F1)
GGAGCGCACUCAGCCACGGUUUACUUCCGUGGCAAUAUUGACCUCNCUCU
AGACAGGUUUCGACCUCUCUGCUAGC
SEQ ID NO 132 (AMX(369)_H1)(ARC1914)
GGAGCGCACUCAGCCACCUGGGAAAAUCUGGGUCCCUGAGUUCUAACAGC
AGAGAUUUUUCGACCUCUCUGCUAGC
SEQ ID NO 133 (AMX(369)_B2)
GGAGCGCACUCNGCCACUUCGGAAUAUCGUUGUCUUCUGGGUGAGCAUGC
GUUGAGGUUUCNACCUCUCUGCUAGC
SEQ ID NO 134 (AMX(369)_G3)
GGAGCGCACUCAGCCACUGGGGAACAUCUCAUGUCUVUGACCGCUCUUGC
AGUAGAAUUUNGACCUCUCUGCUAGC

### Example 2

### Composition and Sequence Optimization and Sequences

### Example 2A

### Minimization

Following a successful selection and following the determination of sequences of aptamers, in addition to determination of functionality in vitro, the sequences were minimized to obtain a shorter oligonucleotide sequence that retained binding specificity to its intended target but had improved binding characteristics, such as improved K_{D} and/or IC₅₀s.

### Example 2A.1

### Minimization of rRfY Clones

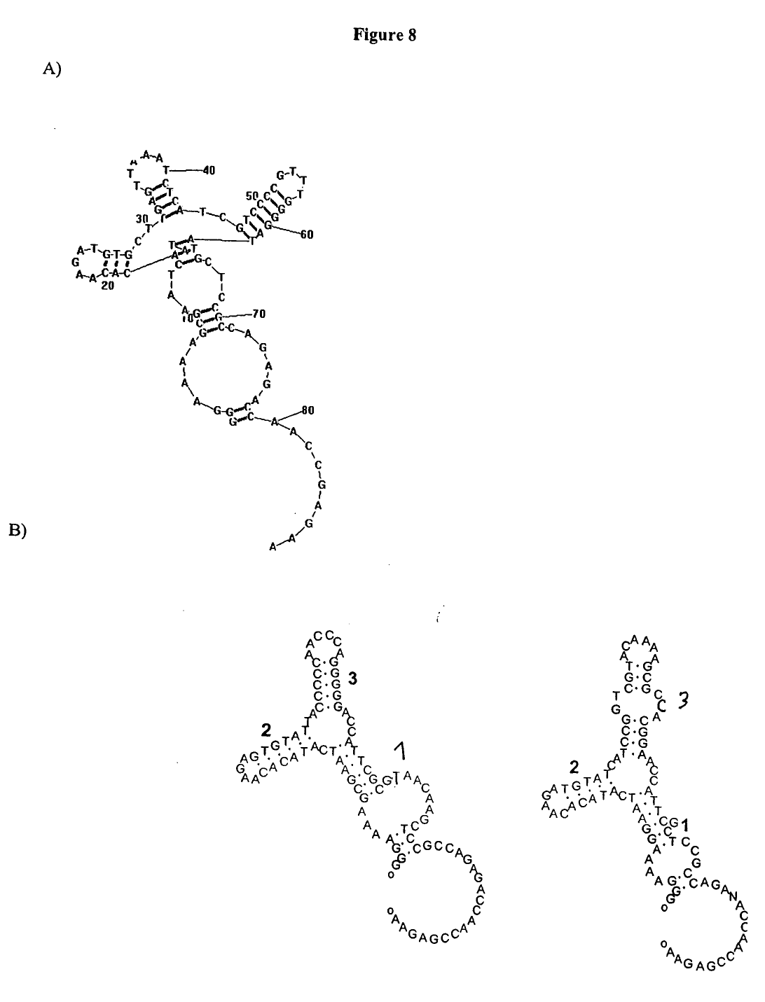

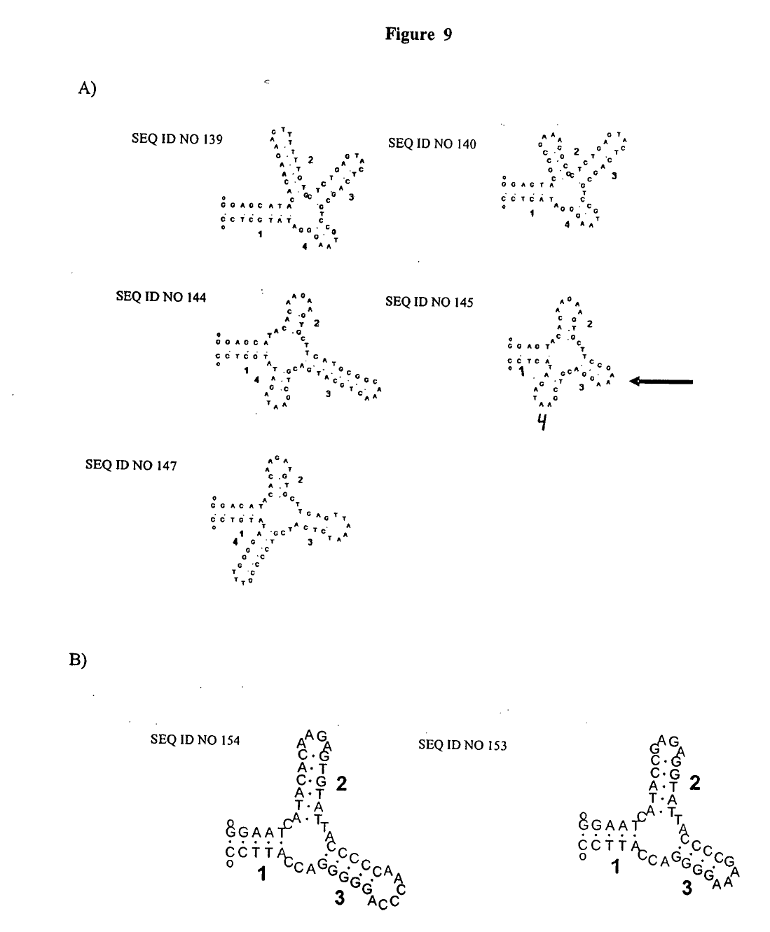

The binding parent clones from the rRfY selection described in Example 1A fell into two principal families of aptamers, referred to as Type 1 and Type 2. FIGS. 8A and 8B show examples of the sequences and predicted secondary structure configurations of Type 1 and Type 2 aptamers. FIGS. 9A and 9B show the minimized aptamer sequences and predicted secondary structure configurations for Types 1 and 2.

On the basis of the IL-23 binding analysis described in Example 1 above and the cell based assay data described in Example 3 below, several Type 1 clones from the rRfY PN-IL-23 selection including AMX84-A10 (SEQ ID NO 43), AMX84-B10 (SEQ ID NO 44), and AMX84-F11 (SEQ ID NO 46) were chosen for further characterization. Minimized DNA construct oligonucleotides were transcribed, gel purified, and tested in dot blot assays for binding to h-IL-23.

The minimized clones A10 min5 (SEQ ID NO 139), A10min6 (SEQ ID NO 140) were based on AMX84-A 10 (SEQ ID NO 43), the minimized clones B10 min4 (SEQ ID NO 144), and B10min5 (SEQ ID NO 145) were based on AMX84-B10 (SEQ ID NO 44), and the minimized clone F11min2 (SEQ ID NO 147), was based on AMX84-F11 (SEQ ID NO 46) (FIG. 9A). The clones were 5′ end labeled with γ-³²P ATP, and were assayed in dot blot assays for K_{D} determination using the same method as for the parent clones. All had significant protein binding (summarized in Table 21), and each was more potent than the respective parent clones from which they are derived when tested in cell based assays as discussed in Example 3 below.

Additionally, minimized constructs exemplifying Type1 and Type 2 aptamers were made and tested based on the consensus sequence of Type 1 and Type 2 aptamer sequence families. Type1.4 (SEQ ID NO 151), and Type1.5 (SEQ ID NO 152) are two examples of such minimized constructs based on the Type 1 family sequence, which displayed high IL-23 binding affinity and the most potent activity in the cell based assay described in Example 3, as compared to the other Type 1 minimers described above.

The resulting rRfY minimers' sequences are listed in Table 20 below. Table 21 shows the minimer binding data for the minimers listed in Table 20.

For the minimized rRfY aptamers described in Table 20 below, the purines (A and G) are 2′-OH purines and the pyrimidines (C and U) are 2′-fluoro pyrimidines. Unless noted otherwise, the individual sequences are represented in the 5′ to 3′ orientation. Each of the sequences listed in Table 20 may be derivatized with polyalkylene glycol (“PAG”) moieties and may or may not contain capping (e.g., a 3′-inverted dT).

TABLE 20
PN-IL-23 2′ F (rRfY) Minimer Aptamer sequences.

SEQ ID NO 135 (A10.min1)
GGAGAUCAUACACAAGAAGUUUUUUGUGCUCUGAGUACUCAGCGUCCGUA
AGGGAUCUCC
SEQ ID NO 136 (A10.min2)
GGAGUCUGAGUACUCAGCGUCCGUAAGGGAUAUGCUCCGCCAGACUCC
SEQ ID NO 137 (A10.min3)
GGAGUUACUCAGCGUCCGUAAGGGAAUAUGCUCCGACUCC
SEQ ID NO 138 (A10.min4)
GGAGUCUGAGUACUCAGCGUCCCGAGAGGGGAUAUGCUCCGCCAGACUCC
SEQ ID NO 139 (A10.min5)
GGAGCAUACACAAGAAGUUUUUUGUGCUCUGAGUACUCAGCGUCCGUAAG
GGAUAUGCUCC
SEQ ID NO 140 (A10.min6)
GGAGUACGCCGAAAGGCGCUCUGAGUACUCAGCGUCCGUAAGGGAUACU
CC
SEQ ID NO 141 (B10.min1)
GGAGCGAAUCAUACACAAGAAGUGCUUCAUGCGGCAAACUGCAUGACGUC
GAAUAGAUAUGCUCC
SEQ ID NO 142 (B10.min2)
GGAUCAUACACAAGAAGUGCUUCAUGCGGCAAACUGCAUGACGUCGAAUA
GAUCC
SEQ ID NO 143 (B10.min3)
GGAUCAUACACAAGAAGUGCUUCACGAAAGUGACGUCGAAUAGAUCC
SEQ ID NO 144 (B10.min4)
GGAGCAUACACAAGAAGUGCUUCAUGCGGCAAACUGCAUGACGUCGAAUA
GAUAUGCUCC
SEQ ID NO 145 (B10.MIN5)
GGAGUACACAAGAAGUGCUUCCGAAAGQACGUCGAAUAGAUACUCC
SEQ ID NO 146 (F11.min1)
GGUUAAAUCUCAUCGUCCCCGUUUGGGGAU
SEQ ID NO 147 (F11.min2)
GGACAUACACAAGAUGUGCUUGAGUUAAAUCUCAUCGUCCCCGUUUGGGG
AUAUGUC
SEQ ID NO 148 (Type1.1)
GGCAUACACGAGAGUGCUGUCGAAAGACUCGGCCGAGAGGCUAUGCC
SEQ ID NO 149 (Type1.2)
GGCAUACGCGAGAGCGCUGGCGAAAGCCUCGGCCGAGAGGCUAUGCC
SEQ ID NO 150 (Type1.3)
GGAUACCCGAGAGGGCUGGCGAAAGCCUCGGCGAGAGCUAUCC
SEQ ID NO 151 (Type1.4)
GGGUACGCCGAAAGGCGCUUCCGAAAGGACGUCCGUAAGGGAUACCC
SEQ ID NO 152 (Type1.5)
GGAGUACGCCGAAAGGCGCUUCCGAAAGGACGUCCGUAAGGGAUACUCC
SEQ ID NO 153 (Type 2.1)
GGAAUCAUACCGAGAGGUAUUACCCGGAAAGGGGACCAUUCC
SEQ ID NO 154 (D9.1)
GGAAUCAUACACAAGAGUGUAUUACCCCCAACCCAGGGGGACCAUUCC
SEQ ID NO 155 (C11.1)
GGAAGAAUGGUCGGAAUCUCUGGCGCCACGCUGAGUAUAGACGGAAGCUC
CGCCAGA
SEQ ID NO 156 (C11.2)
GGAGGCGCCACGCUGAGUAUAGACGGAAGCUCCGCCUCC
SEQ ID NO 157 (C10.1)
GGACACAAGAGAUGUAUUCAGGCGGUCCGCAUUGAUGUCAGUUAUGCGUA
GCUCCGCC
SEQ ID NO 158 (C10.2)
GGCGGUCCGCAUUGAUGUCAGUUAUGCGUAGCUCCGCC

TABLE 21
PN-IL-23 rRfY Minimer Binding data

| SEQ ID | Clone | +/−IL-23 | | IL-23 K_{D} |
|---|---|---|---|---|
| No. | Description | 20 nM | +/−IL-23 100 nM | (nM) |
| 135 | A10min1 | 2.2 | 3.1 | |
| 136 | A10min2 | 4.4 | 6.0 | |
| 137 | A10min3 | 0.8 | 1.6 | |
| 138 | A10min4 | 0.9 | 0.7 | |
| 146 | F11min1 | 0.8 | 0.6 | |
| 147 | F11min2 | 7.8 | 16.9 | 65 |
| 141 | B10min1 | 7.5 | 33.9 | |
| 142 | B10min2 | 1.3 | 1.6 | |
| 143 | B10min3 | 0.6 | 0.8 | |
| 139 | A10min5 | 12.8 | 40.9 | 57.8 |
| 140 | A10min6 | 13.6 | 41.7 | 48.3 |
| 144 | B10min4 | 39.4 | 122.1 | 36.4 |
| 145 | B10min5 | 20.7 | 89.2 | 276.9 |
| 148 | IL-23 Type 1.1 | 1.4 | 0.9 | |
| 149 | IL-23 Type 1.2 | 0.8 | 0.7 | |
| 150 | IL-23 Type 1.3 | 0.8 | 0.6 | |
| 153 | IL-23 Type 2.1 | 1.7 | 5.2 | |
| 154 | D9.1 | 1.2 | 3.9 | |
| 155 | C11.1 | 1.0 | 3.5 | |
| 156 | C11.2 | 1.1 | 2.3 | |
| 157 | C10.1 | 1.4 | 4.4 | |
| 158 | C10.2 | 1.4 | 1.5 | |
| 151 | IL-23 Type 1.4 | 2.3 | 11.7 | 185.3 |
| 152 | IL-23 Type 1.5 | 5.2 | 26.9 | 31.4 |

**Assays performed + 0.1 mg/mL tRNA, 30 min RT incubation
**R&D IL-23 (carrier free protein)

### Example 2A.2

### Minimization of dRmY Selection 1

Following the dRmY selection process for aptamers binding to IL-23 (described in Example 1C above) and determination of the oligonucleotide sequences, the sequences were systematically minimized to obtain shorter oligonucleotide sequences that retain the binding characteristics. On the basis of the IL-23 binding analysis described in Example 1A above and the cell based assay data described in Example 3 below, ARC489 (SEQ ID NO 91) (74mer) was chosen for further characterization. 3 minimized constructs based on clone ARC489 (SEQ ID NO 91) were designed and generated. The clones were 5′ end labeled with γ-³²P ATP, and were assayed in dot blot assays for K_{D} determination using the same method as for the parent clones in 1×PBS+0.1 mg/mL tRNA, 0.1 mg/mL salmon sperm DNA, 0.1 mg/mL BSA, for a 30 minute incubation at room temperature. Table 22 shows the sequences for the minimized dRmY aptamers. Table 23 includes the binding data for the dRmY minimized aptamers. Only one minimized clone, ARC527 (SEQ ID NO 159), showed binding to IL-23. This clone was tested in the TransAM™ STAT3 activation assay described in Example 3 below, and showed a decrease in assay activity compared to its respective parent, ARC489 (SEQ ID NO 91).

For the minimized dRmY aptamers described in Table 22 below, the purines (A and G) are deoxy-purines and the pyrimidines (U and C) are 2′-OMe pyrimidines. Unless noted otherwise, the individual sequences are represented in the 5′ to 3′ orientation. Each of the sequences listed in Table 22 may be derivatized with polyalkylene glycol (“PAG”) moieties and may or may not contain capping (e.g., a 3′-inverted dT).

TABLE 22
Sequences of dRmY Minimized

SEQ ID NO 159 (ARC527)
ACAGCGCCGGUGGGCGGGCAUUGGGUGGAUGCGCUGU
SEQ ID NO 160 (ARC528)
GCGCCGGUGGGCGGGCACCGGGUGGAUGCGCC
SEQ ID NO 161 (ARC529)
ACAGCGCCGGUGUUUUCAUUGGGUGGAUGCGCUGU

TABLE 23
Binding characterization of dRmY selection 1 minimers

| SEQ ID NO | Clone Name | K_{D} (nM) |
|---|---|---|
| SEQ ID 159 | ARC 527 | 12.6 |
| SEQ ID 160 | ARC 528 | NB |
| SEQ ID 161 | ARC 529 | NB |

**R&D IL-23 (carrier free protein)
N.B. = no binding detectable

### Example 2A.3

### Minimization of dRmY Selection 2

Following the dRmY selection process for aptamers binding to IL-23 (described in Example 1D above) and determination of the oligonucleotide sequences, the sequences were systematically minimized to obtain shorter oligonucleotide sequences that retain the binding characteristics

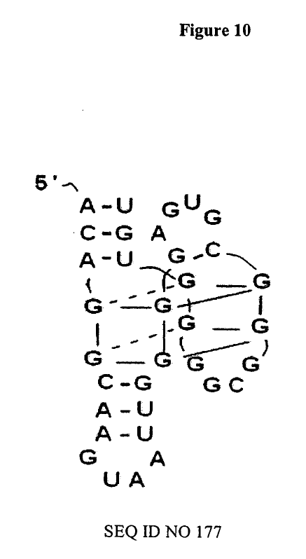

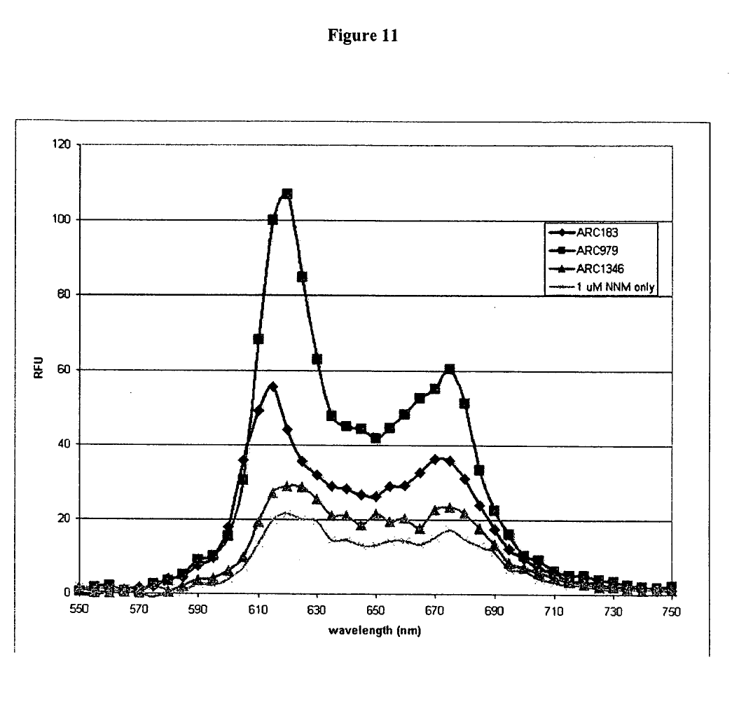

Based on sequence analysis and visual inspection of the parent dRmY aptamer sequences described in Example 1D, it was hypothesized that the active conformation of dRmY h-IL-23 binding clones and their minimized constructs fold into a G-quartet structure (FIG. 10). Analysis of the functional binding sequences revealed a pattern of G doubles consistent with a G quartet formation (Table 24). The sequences within the G quartet family fell into 2 subclasses, those with 3 base pairs in the 1st stem and those with 2. It has been reported that in much the same way that ethidium bromide fluorescence is increased upon binding to duplex RNA and DNA, that N-methylmesoporphyrin IX (NMM) fluorescence is increased upon binding to G-quartet structures (Arthanari et al., Nucleic Acids Research, 26(16): 3724 (1996); Marathais et al., Nucleic Acids Research, 28(9): 1969 (2000); Joyce et al., Applied Spectroscopy, 58(7): 831 (2004)). Thus as shown in FIG. 11, NMM fluorescence was used to confirm that ARC979 (SEQ ID NO 177) does in fact adopt a G-quartet structure. According to the literature protocols, 100 microliter reactions containing ˜1 micromolar NMM and ˜2 micromolar aptamer in Dulbecco's PBS containing magnesium and calcium were analyzed using a SpectraMax Gemini XS fluorescence plate reader. Fluorescence emission spectra were collected from 550 to 750 nm with and excitation wavelength of 405 nm. The G-quartet structure of the anti-thrombin DNA aptamer ARC183 (Macaya et al., Proc. Natl. Acad. Sci., 90: 3745 (1993)) was used as a positive control in this experiment. ARC1346 is an aptamer of a similar size and nucleotide composition as ARC979 (SEQ ID NO 177) that is not predicted to have a G-quartet structure and was used as a negative control in the experiment. As can be seen in FIG. 11, ARC183 and ARC979 (SEQ ID NO 177) show a significant increase in NMM fluorescence relative to NMM alone while the negative control, ARC1346 does not.

Minimized constructs were synthesized on an ABI EXPEDITE™ DNA synthesizer, then deprotected by standard methods. The minimized clones were gel purified on a 10% PAGE gel, and the RNA was passively eluted in 300 mM NaOAc and 20 mM EDTA, followed by ethanol precipitation.

The clones were 5′ end labeled with γ-³²P ATP, and were assayed in dot blot assays for K_{D} determination using the direct binding assay in which the aptamer was radio-labeled and held at a trace concentration (<90 pM) while the concentration of IL-23 was varied, in 1×PBS with 0.1 mg/mL BSA, for a 30 minute incubation at room temperature. The fraction aptamer bound vs. [IL-23] was used to calculate the K_{D} by fitting the following equation to the data:

Fraction aptamer bound=amplitude*([IL-23]/(K_{D}+[IL-23]))+background binding.

Several of the minimized constructs from the dRmY Selection 2 were also assayed in a competition format in which cold aptamer was titrated and competed away trace ³²P ATP labeled aptamer In the competition assay, the [IL-23] was held constant, the [trace labeled aptamer] was held constant, and the [unlabeled aptamer] was varied. The K_{D} was calculated by fitting the following equation to the data:

Fraction aptamer bound=amplitude*([aptamer]/(K_{D}+[aptamer]))+background binding.

Minimers based upon the G quartet were functional binders, whereas minimers based on a folding algorithm that predicts stem loops (RNAstructure; D. H. Mathews, et al., “Expanded Sequence Dependence of Thermodynamic Parameters Improves Prediction of RNA Secondary Structure”. Journal of Molecular Biology, 288, 911-940, (1999)) and that did not contain the pattern of G doubles were non functional (ARC793 (SEQ ID NO 163)).

Table 25 below summarizes the minimized sequences and the parent clone from which they were derived, and Table 26 summarizes the binding characterization from direct binding assays (+/−tRNA) and competition binding assays for the minimized constructs tested.

TABLE 24
Alignment of functional clones. (only the regions
within the G quartet are represented)

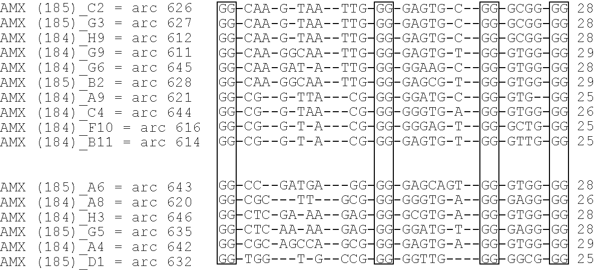

The SEQ ID NOS for the clones listed in Table 24 are found in Table 12.

For the minimized dRmY aptamers described in Table 25 below, the purines (A and G) are deoxy-purines and the pyrimidines (C and U) are 2′-OMe pyrimidines. Unless noted otherwise, the individual sequences are represented in the 5′ to 3′ orientation. Each of the sequences listed in Table 25 may be derivatized with polyalkylene glycol (“PAG”) moieties and may or may not contain capping (e.g., a 3′-inverted dT).

TABLE 25
dRmY minimer sequences

| SEQ | | | |
|---|---|---|---|
| ID | Parent | | |
| NO | Clone | Minimer | Minimized Sequence |
| 162 | ARC627 | ARC792 | GGCAAGUAAUUGGGGAGUGCGGGCGGGG |
| 163 | ARC614 | ARC793 | CUACAAGGCGGUACGGGGAGUGUGG |
| 164 | ARC614 | ARC794 | GGCGGUACGGGGAGUGUGGGUUGGGGCCGG |
| 165 | ARC616 | ARC795 | CGAUAUAGGCGGUACGGGGGGAGUGGGCUGGG |
| | | | GUCG |
| 166 | ARC626 | ARC796 | UAAUUGGGGAGUGCGGGCGGGGGGUCGAUCG |
| 167 | ARC626 | ARC797 | GGUGGGGAGUGCGGGCGGGGGGUCGCC |
| 168 | ARC627 | ARC889 | ACAGGCAAGGUAAUUGGGGAGUGCGGGCGGGG |
| | | | UGU |
| 169 | ARC627 | ARC890 | CCAGGCAAGGUAAUUGGGGAGUGCGGGCGGGG |
| | | | UGG |
| 170 | ARC627 | ARC891 | GGCAAGGUAAUUGGGAAGUGUGGGCGGGG |
| 171 | ARC627 | ARC892 | GGCAAGGUAAUUGGGUAGUGAGGGCGGGG |
| 172 | ARC627 | ARC893 | GGCAAGGUAAUUGGGGAGUGCGGGCUGGG |
| 173 | ARC627 | ARC894 | GGCAAGGUAAUUGGGAAGUGUGGGCUGGG |
| 174 | ARC627 | ARC895 | GGCAAGGUAAUUGGGUAGUGAGGGCUGGG |
| 175 | ARC627 | ARC896 | ACAGGCAAGGUAAUUGGGUAGUGAGGGCUGGG |
| | | | UGU |
| 176 | ARC627 | ARC897 | GAUGUUGGCAAGUAAUUGGGGAGUGCGGGCGG |
| | | | GGUUCAUC-3T |
| 177 | ARC627 | ARC979 | ACAGGCAAGUAAUUGGGGAGUGCGGGCGGGGU |
| | | | GU |
| 178 | ARC627 | ARC980 | CCAGGCAAGUAAUUGGGGAGUGCGGGCGGGGU |
| | | | GG |
| 179 | ARC621 | ARC981 | GGCGGUUACGGGGGAUGCGGGUGGG |
| 180 | ARC621 | ARC982 | GGCGGUUACGGGGGAUGCGGGUGGGACAGG |
| 181 | ARC627 | ARC1117 | GGCAAGUAAUUGGGGAGUGCGGGCGG |
| 182 | ARC627 | ARC1118 | ACAGGCAAGUAAUUGGGGAGUGCGGGCGGUGU |
| 183 | ARC614 | ARC1119 | GGCGGUACGGGGAGUGUGGGUUGGGGCC |
| 184 | ARC614 | ARC1120 | GGCGGUACGGGGAGUGUGGGCUGGGGCC |
| 185 | ARC614 | ARC1121 | GGUACGGGGAGUGUGGGUUGGG |
| 186 | ARC614 | ARC1122 | GGUACGGGGAGUGUGGGCUGGG |
| 187 | ARC614 | ARC1123 | GGCGGUACGGGGAGUGUGGGUUGGGCC |
| 188 | ARC614 | ARC1124 | GGCGGUACGGGGAGUGUGGGCUGGGCC |
| 189 | ARC614 | ARC1125 | GGUACGGGGAGUGUGGGUUGG |
| 190 | ARC614 | ARC1126 | GGUACGGGGAGUGUGGGCUGG |
| 191 | ARC616 | ARC1127 | GGCGGUACGGGGGGAGUGGGCUGGGGUC |
| 192 | ARC616 | ARC1128 | GGCGGUACGGGGGGAGUGGGCUGGGUC |
| 193 | ARC616 | ARC1129 | GGCGGUACGGGGAGAGUGGGCUGGGGUC |
| 194 | ARC616 | ARC1130 | GGUACGGGGGGAGUGGGCUGGG |
| 195 | ARC616 | ARC1131 | GGUACGGGGGGAGUGGGCUGG |
| 196 | ARC616 | ARC1132 | GGUACGGGGAGAGUGGGCUGGG |
| 197 | ARC616 | ARC1170 | GGCGGUACGGGGGGAGUGGGCUGGG |
| 198 | ARC614 | ARC1171 | GGCGGUACGGGGAGUGUGGGUUGGG |

TABLE 26
protein binding characterization of dRmY minimers

| SEQ | | K_{D} | K_{D} | K_{D} |
|---|---|---|---|---|
| ID | Minimer | (+tRNA) | (−tRNA) | (competition) |
| NO | ARC# | nM | nM | nM |
| 162 | ARC792 | 117 | | 11 |
| 164 | ARC794 | 69 | | 14 |
| 165 | ARC795 | 40 | | 4 |
| 166 | ARC796 | 106 | | |
| 167 | ARC797 | 50 | | |
| 168 | ARC889 | 115 | | |
| 169 | ARC890 | 114 | | |
| 170 | ARC891 | 177 | | |
| 171 | ARC892 | 255 | | |
| 172 | ARC893 | 2857 | | |
| 173 | ARC894 | no binding | | |
| 174 | ARC895 | no binding | | |
| 175 | ARC896 | no binding | | |
| 176 | ARC897 | 93 | | |
| 177 | ARC979 | 93 | 90 | 9 |
| 178 | ARC980 | 139 | | |
| 179 | ARC981 | no binding | | |
| 180 | ARC982 | no binding | | |
| 181 | ARC1117 | <parent | | |
| | | clone | | |
| 182 | ARC1118 | <parent | | |
| | | clone | | |
| 183 | ARC1119 | <parent | | |
| | | clone | | |
| 184 | ARC1120 | <parent | | |
| | | clone | | |
| 185 | ARC1121 | <parent | | |
| | | clone | | |
| 186 | ARC1122 | <parent | | |
| | | clone | | |
| 187 | ARC1123 | <parent | | |
| | | clone | | |
| 188 | ARC1124 | <parent | | |
| | | clone | | |
| 189 | ARC1125 | <parent | | |
| | | clone | | |
| 190 | ARC1126 | <parent | | |
| | | clone | | |
| 191 | ARC1127 | <parent | | |
| | | clone | | |
| 192 | ARC1128 | <parent | | |
| | | clone | | |
| 193 | ARC1129 | <parent | | |
| | | clone | | |
| 194 | ARC1130 | <parent | | |
| | | clone | | |
| 195 | ARC1131 | <parent | | |
| | | clone | | |
| 196 | ARC1132 | <parent | | |
| | | clone | | |
| 197 | ARC1170 | no binding | | |
| 198 | ARC1171 | no binding | | |

The competitive binding data was re-analyzed in a saturation binding experiment where the concentration of ligand (aptamer) was varied and the concentration of receptor (IL-23) was held constant and the [bound aptamer] was plotted versus the [total input aptamer]. ARC979 (SEQ ID NO 177) was used in this analysis.

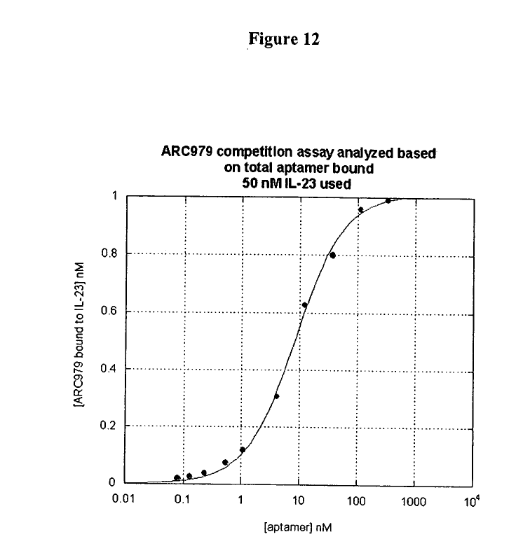

The [ARC979] bound saturated at ˜1.7 nM (FIG. 12), which suggested that the concentration of IL-23 that was competent to bind aptamer was 1 nM, or 2% ( 1/50) of the input IL-23. The calculated K_{D} value was 8 nM, which agreed well with the value obtained by fitting the data represented in competition mode (8.7 nM).

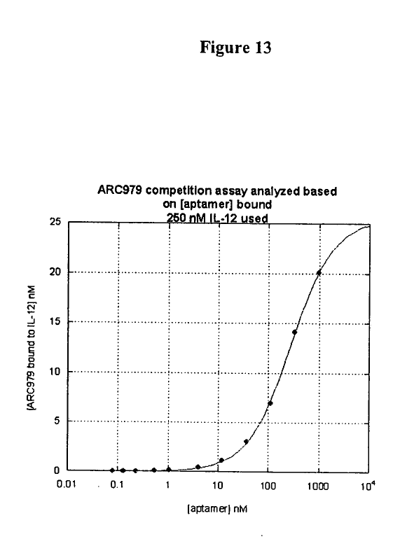

When IL-12 competition binding data was subjected to the same analysis (FIG. 13), the fraction active IL-12 was higher (10%), and the specificity of ARC979 for IL-23 vs. IL-12 (33-fold) was greater than what was predicted by the direct binding measurements (2-5 fold).

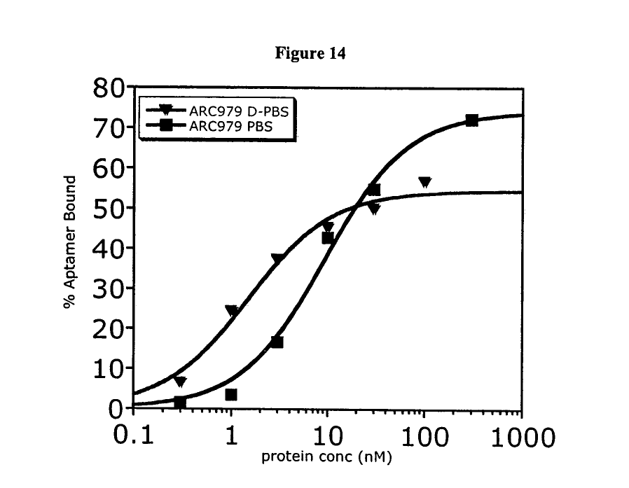

Subsequently, the direct binding assay was repeated for ARC979 using the binding reaction conditions described previously (1×PBS with 0.1 mg/mL BSA for 30 minute incubation at room temperature) and using different binding reaction conditions (1× Dulbecco's PBS (with Mg⁺⁺ and Ca⁺⁺) with 0.1 mg/mL BSA for 30 minutes at room temperature). In both, newly chemically synthesized aptamers were purified using denaturing polyacrylamide gel electrophoresis, 5′ end labeled with γ-³²P ATP and were tested for direct binding to full human IL-23. An 8 point protein titration was used in the dot blot binding assay (either {100 nM, 30 nM, 10 nM, 3 mM, 1 nM, 300 pM, 100 pM, 0 pM} or {10 nM, 3 nM, 1 nM, 300 pM, 100 pM, 30 pM, 10 pM, 0 pM}). K_{D} values were calculated by fitting the equation y=(max/(1+K/protein))+yint using KaleidaGraph (KaleidaGraph v.3.51, Synergy Software). The buffer conditions appeared to affect the binding affinity somewhat. Under the 1×PBS condition, the K_{D} value for ARC979 was calculated to be ˜10 nM, whereas under the 1× Dulbecco's PBS condition, the K_{D} value for ARC979 was calculated to be ˜1 nM. (see FIG. 14). These K_{D} values were verified in subsequent assays (data not shown), and are consistent with the IC₅₀ value of ˜6 nM that ARC979 yields in the PHA Blast assay described below in Example 3D.

### Example 2A.4

### Mouse IL-23 rRfY Minimization

Based on visual inspection of the parent clone sequences of the mouse IL-23 rRfY aptamers described in Example 1E, and predicted RNA structures using an RNA folding program (RNAstructure), minimized constructs were designed for each of the seven binding mIL-23 clones. PCR templates for the minimized construct oligos were ordered from Integrated DNA Technologies (Coraville, Iowa). Constructs were PCR amplified, transcribed, gel purified, and tested for binding to mIL-23 using the dot blot binding assay previously described. Trace ³²P-labeled RNA was combined with mIL-23 and incubated at room temperature for 30 min in 1×PBS plus 0.1 mg/mL BSA for a final volume of 30 μL. The reaction was added to a dot blot apparatus (Schleicher and Schuell Minifold-1 Dot Blot, Acrylic). Binding curves were generated as described in previous sections. Table 32 lists the sequences of the mIL-23 binding minimized constructs. Table 33 summarizes the protein binding characterization for each rRfY minimized construct that had significant binding to mIL-23.

Unless noted otherwise, individual sequences listed below are represented in the 5′ to 3′ orientation and represent the sequences that bind to mouse IL-23 selected under rRfY SELEX™ conditions wherein the purines (A and G) are 2′-OH and the pyrimidines (U and C) are 2′-fluoro. Each of the sequences listed in Table 32 may be derivatized with polyalkylene glycol (“PAG”) moieties and may or may not contain capping (e.g., a 3′-inverted dT).

TABLE 32
minimized mouse rRfY clone sequences

SEQ ID NO 199 (ARC 1739)
GGGCACUCAGCCACAGGUGGCUUAAUACUGUAAAGACGUGCCC
SEQ ID NO 200 (ARC 1918)
GGAGCGCACUCAGCCACCGGCUUAAUAUCCAAUAGGAACGUUCGCUCU
SEQ ID NO 201
GGGCACUCAGCCACAGCUCGGUGGCUUAAUAUCUAUGUGAACGUGCCC
SEQ ID NO 202
GGGCACUCAGCCACCUUGGGCUUAAUACCUAUCGGAUGUGCCC

TABLE 33
mIL-23 rRfY Clone K_{D} Summary

| Minimized | | Parent | |
|---|---|---|---|
| Clone | Parent Clone | Clone | K_{D} mIL-23 |
| SEQ ID NO | Name | SEQ ID NO | (nM) |
| 199 | ARC1628 | 124 | 1 |
| 200 | ARC1632 | 128 | 1 |
| 201 | ARC1633 | 129 | 25 |
| 202 | ARC1634 | 130 | 19 |

*30 min RT incubation for K_{D} determination
*1X BS + 0.1 mg/mL BSA reaction buffer

### Example 2B

### Optimization Through Medicinal Chemistry

Aptamer Medicinal Chemistry is an aptamer improvement technique in which sets of variant aptamers are chemically synthesized. These sets of variants typically differ from the parent aptamer by the introduction of a single substituent, and differ from each other by the location of this substituent. These variants are then compared to each other and to the parent. Improvements in characteristics may be profound enough that the inclusion of a single substituent may be all that is necessary to achieve a particular therapeutic criterion.

Alternatively the information gleaned from the set of single variants may be used to design further sets of variants in which more than one substituent is introduced simultaneously. In one design strategy, all of the single substituent variants are ranked, the top 4 are chosen and all possible double (6), triple (4) and quadruple (1) combinations of these 4 single substituent variants are synthesized and assayed. In a second design strategy, the best single substituent variant is considered to be the new parent and all possible double substituent variants that include this highest-ranked single substituent variant are synthesized and assayed. Other strategies may be used, and these strategies may be applied repeatedly such that the number of substituents is gradually increased while continuing to identify further-improved variants.

Aptamer Medicinal Chemistry is most valuable as a method to explore the local, rather than the global, introduction of substituents. Because aptamers are discovered within libraries that are generated by transcription, any substituents that are introduced during the SELEX™ process must be introduced globally. For example, if it is desired to introduce phosphorothioate linkages between nucleotides then they can only be introduced at every A (or every G, C, T, U etc.) (globally substituted). Aptamers which require phosphorothioates at some As (or some G, C, T, U etc.) (locally substituted) but cannot tolerate it at other As cannot be readily discovered by this process.

The kinds of substituent that can be utilized by the Aptamer Medicinal Chemistry process are only limited by the ability to generate them as solid-phase synthesis reagents and introduce them into an oligomer synthesis scheme. The process is certainly not limited to nucleotides alone. Aptamer Medicinal Chemistry schemes may include substituents that introduce steric bulk, hydrophobicity, hydrophilicity, lipophilicity, lipophobicity, positive charge, negative charge, neutral charge, zwitterions, polarizability, nuclease-resistance, conformational rigidity, conformational flexibility, protein-binding characteristics, mass etc. Aptamer Medicinal Chemistry schemes may include base-modifications, sugar-modifications or phosphodiester linkage-modifications.

When considering the kinds of substituents that are likely to be beneficial within the context of a therapeutic aptamer, it may be desirable to introduce substitutions that fall into one or more of the following categories:
(1) Substituents already present in the body, e.g., 2′-deoxy, 2′-ribo, 2′-O-methyl purines or pyrimidines or 5-methyl cytosine.
(2) Substituents already part of an approved therapeutic, e.g., phosphorothioate-linked oligonucleotides.
(3) Substituents that hydrolyze or degrade to one of the above two categories, e.g., methylphosphonate-linked oligonucleotides.

### Example 2B.1

### Optimization of ARC979 by Phosphorothioate Substitution

ARC979 (SEQ ID NO 177) is a 34 nucleotide aptamer to IL-23 of dRmY composition. 21 phosphorothioate derivatives of ARC979 were designed and synthesized in which single phosphorothioate substitutions were made at each phosphate linkage (ARC1149 to ARC1169) (SEQ ID NO 203 to SEQ ID NO 223) (see Table 27). These molecules were gel purified and assayed for IL-23 binding using the dot blot assay as described above and compared to each other and to the parent molecule, ARC979. An 8 point IL-23 titration (0 nM to 300 nM, 3 fold serial dilutions) was used in the binding assay. Calculated KDS are summarized in Table 28.

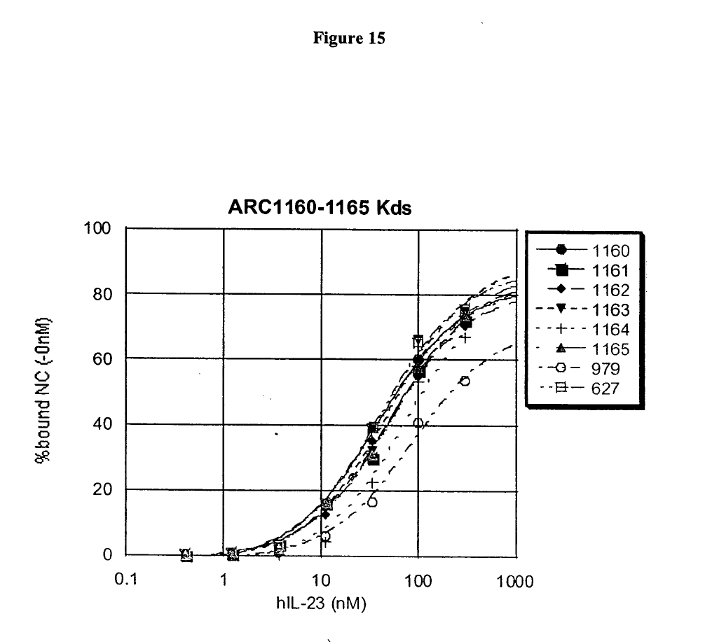

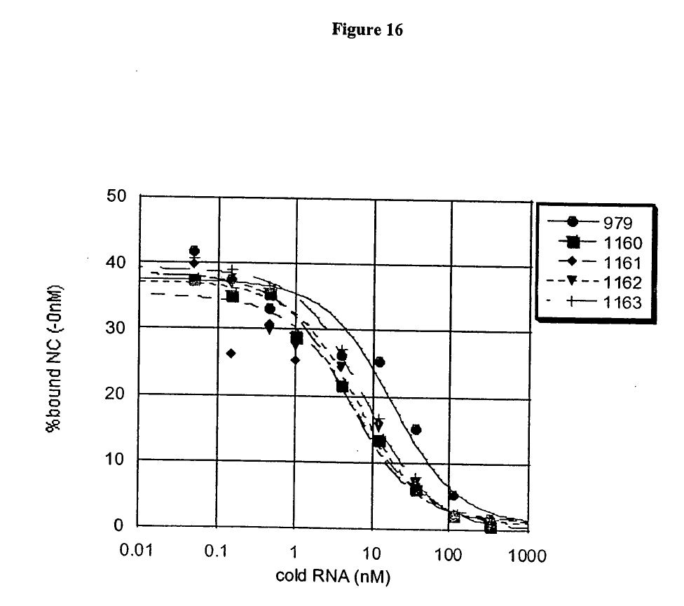

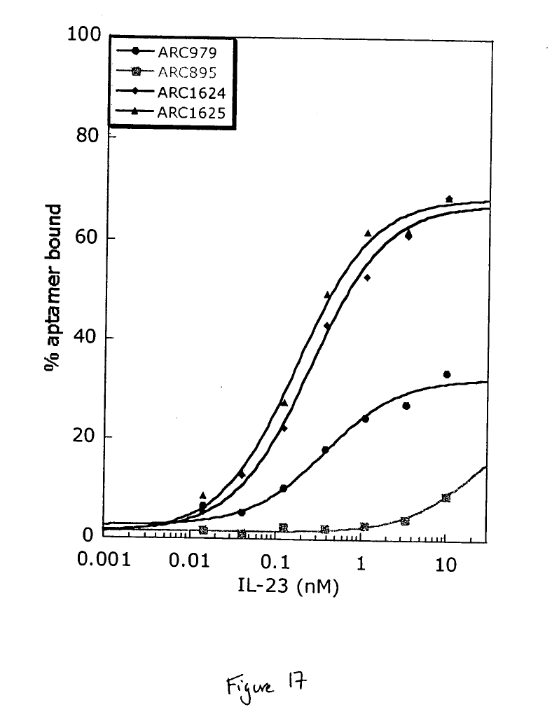

The inclusion of phosphorothioate linkages in ARC979 was well tolerated when compared to ARC979. Many of these constructs have an increased proportion binding to IL-23 and additionally have improved (i.e., lower) K_{D} values (FIG. 15). A similar increase in affinity is seen in competition assays (FIG. 16), which further supports that the phosphorothioate derivatives of ARC979 compete for IL-23 at a higher affinity than ARC979.

Unless noted otherwise, each of the sequences listed in Table 27 below are in the 5′-3′ direction, may be derivatized with polyalkylene glycol (“PAG”) moieties, and may or may not contain capping (e.g., a 3′-inverted dT).

TABLE 27

Sequences of ARC979 phosphorothioate derivatives:
Single Phosphorothioate substitutions

| | | | |
|---|---|---|---|
| SEQ | | Phosphorothiote | |
| ID | | linker between | |
| NO | ARC# | bases (x, y) | Sequence |

| | | | | |
|---|---|---|---|---|
| 203 | ARC1149 | 1 | 2 | ACAGGCAAGUAAUUGGGGAGUGCGGGCGGGGUGU |
| 204 | ARC1150 | 2 | 3 | ACAGGCAAGUAAUUGGGGAGUGCGGGCGGGGUGU |
| 205 | ARC1151 | 6 | 7 | ACAGGCAAGUAAUUGGGGAGUGCGGGCGGGGUGU |
| 206 | ARC1152 | 7 | 8 | ACAGGCAAGUAAUUGGGGAGUGCGGGCGGGGUGU |
| 207 | ARC1153 | 8 | 9 | ACAGGCAAGUAAUUGGGGAGUGCGGGCGGGGUGU |
| 208 | ARC1154 | 9 | 10 | ACAGGCAAGUAAUUGGGGAGUGCGGGCGGGGUGU |
| 209 | ARC1155 | 10 | 11 | ACAGGCAAGUAAUUGGGGAGUGCGGGCGGGGUGU |
| 210 | ARC1156 | 11 | 12 | ACAGGCAAGUAAUUGGGGAGUGCGGGCGGGGUGU |
| 211 | ARC1157 | 12 | 13 | ACAGGCAAGUAAUUGGGGAGUGCGGGCGGGGUGU |
| 212 | ARC1158 | 13 | 14 | ACAGGCAAGUAAUUGGGGAGUGCGGGCGGGGUGU |
| 213 | ARC1159 | 14 | 15 | ACAGGCAAGUAAUUGGGGAGUGCGGGCGGGGUGU |
| 214 | ARC1160 | 18 | 19 | ACAGGCAAGUAAUUGGGGAGUGCGGGCGGGGUGU |
| 215 | ARC1161 | 19 | 20 | ACAGGCAAGUAAUUGGGGAGUGCGGGCGGGGUGU |
| 216 | ARC1162 | 20 | 21 | ACAGGCAAGUAAUUGGGGAGUGCGGGCGGGGUGU |
| 217 | ARC1163 | 21 | 22 | ACAGGCAAGUAAUUGGGGAGUGCGGGCGGGGUGU |
| 218 | ARC1164 | 22 | 23 | ACAGGCAAGUAAUUGGGGAGUGCGGGCGGGGUGU |
| 219 | ARC1165 | 26 | 27 | ACAGGCAAGUAAUUGGGGAGUGCGGGCGGGGUGU |
| 220 | ARC1166 | 27 | 28 | ACAGGCAAGUAAUUGGGGAGUGCGGGCGGGGUGU |
| 221 | ARC1167 | 28 | 29 | ACAGGCAAGUAAUUGGGGAGUGCGGGCGGGGUGU |
| 222 | ARC1168 | 32 | 33 | ACAGGCAAGUAAUUGGGGAGUGCGGGCGGGGUGU |
| 223 | ARC1169 | 33 | 34 | ACAGGCAAGUAAUUGGGGAGUGCGGGCGGGGUGU |

TABLE 28
K_{D} summary for ARC979 phopsphorothioate derivatives

| SEQ | | K_{D} | | K_{D} |
|---|---|---|---|---|
| ID | | (+tRNA) | K_{D} (−tRNA) | (competition) |
| NO | ARC# | nM | nM | nM |
| 177 | ARC979 | 93 | 90 | 9 |
| 203 | ARC1149 | not tested | | |
| 204 | ARC1150 | not tested | | |
| 205 | ARC1151 | | 142 | |
| 206 | ARC1152 | | 232 | |
| 207 | ARC1153 | | 174 | |
| 208 | ARC1154 | | 412 | |
| 209 | ARC1155 | | 168 | |
| 210 | ARC1156 | | 369 | |
| 211 | ARC1157 | | 69 | |
| 212 | ARC1158 | | 192 | |
| 213 | ARC1159 | | 77 | |
| 214 | ARC1160 | | 38 | 5 |
| 215 | ARC1161 | | 55 | 6 |
| 216 | ARC1162 | | 47 | 6 |
| 217 | ARC1163 | | 49 | 8 |
| 218 | ARC1164 | | 79 | |
| 219 | ARC1165 | | 55 | |
| 220 | ARC1166 | | 132 | |
| 221 | ARC1167 | | 107 | |
| 222 | ARC1168 | | 82 | |
| 223 | ARC1169 | | 74 | |

### Example 2B.2

### Optimization

### 2′-OMe, Phosphorothioate and Inosine Substitutions

Systematic modifications were made to ARC979 (SEQ ID NO 177) to increase overall stability and plasma nuclease resistance. The most stable and potent variant of ARC979 was identified through a systematic synthetic approach involving 4 phases of aptamer synthesis, purification and assay for binding activity. The first step in the process was the synthesis and assay for binding activity of ARC1386 (SEQ ID NO 224) (ARC979 with a 3′-inverted-dT). Once ARC1386 (SEQ ID NO 224) was shown to bind to IL-23 with an affinity similar to that of the parent molecule ARC979 (SEQ ID NO 177), all subsequent derivatives of ARC979 were synthesized with a stabilizing 3′-inverted-dT.

The dot blot binding assay previously described was used to characterize the relative potency of the majority of the aptamers synthesized. For K_{D} determination, chemically synthesized aptamers were purified using denaturing polyacrylamide gel electrophoresis, 5′ end labeled with γ-³²P ATP and were tested for direct binding to full human IL-23. An 8 point protein titration was used in the dot blot binding assay (either {100 nM, 30 nM, 10 nM, 3 nM, 1 nM, 300 pM, 100 pM, 0 pM} or {10 nM, 3 nM, 1 nM, 300 pM, 100 pM, 30 pM, 10 pM, 0 pM}) in Dulbecco's PBS (with Mg⁺⁺ and Ca⁺⁺) with 0.1 mg/mL BSA. K_{D} values were calculated by fitting the equation y=(max/(1+K/protein))+yint using KaleidaGraph (KaleidaGraph v. 3.51, Synergy Software). Sequences of the ARC979 derivatives synthesized, purified and assayed for binding to IL-23 as well as the results of the protein binding characterization are tabulated below in Tables 29 and 30. As can be seen in Table 30, and as previously described in Example 2A.3 above, ARC1386 (SEQ ID NO 224) (which is ARC979 (SEQ ID NO 177) with a 3′ inverted dT) has a K_{D} of 1 nM under these conditions.

In phase 1 of the optimization process, comprised of ARC1427-ARC1471 (SEQ ID NOs 225-269), each individual purine residue in ARC1386 (SEQ ID NO 224) was replaced by the corresponding 2′-O methyl containing residue. Additionally in phase 1, a series of individual and composite phosphorothioate substitutions were tested based on results generated previously which had suggested that in addition to conferring nuclease stability, phosphorothioate substitutions enhanced the binding affinity of derivatives of ARC979. Finally at the end of phase 1, a series of aptamers were tested that explored further the role of stem 1 in the functional context of ARC979/ARC1386. As seen from the binding data in Table 30, many positions readily tolerated substitution of a deoxy residue for a 2′-O methyl residue. Addition of any particular phosphorothioate did not appear to confer a significant enhancement in the affinity of the aptamers. Interestingly, as can be seen by comparison of ARC1465-1471 (SEQ ID NOs 263-269), stem 1 was important for maintenance of high affinity binding, however its role appeared to be a structural clamp since introduction of PEG spacers between the aptamer core and the 2 strands that comprise stem 1 did not appear to significantly impact the binding properties of the aptamers.

Based upon the structure activity relationship (SAR) results of the from phase 1 of the optimization process, a second series of aptamers were designed, synthesized, purified and tested for binding to IL-23. In phase 2 optimization, comprised of ARC1539-ARC1545 (SEQ ID NOs 270-276), the data from phase 1 was used to generate more highly modified composite molecules using exclusively 2′-O methyl substitutions. For these and all subsequent molecules, the goal was to identify molecules that retained an affinity (K_{D}) of 2 nM or better as well as an extent of binding at 100 nM (or 10 nM in phases 3 and 4) IL-23 of at least 50%. The best of these in terms of simple binding affinity was ARC1544 (SEQ ID NO 275).

In phase 3 of optimization, comprised of ARC1591-ARC1626 (SEQ ID NOs 277-312), the stability of the G-quartet structure of ARC979 (SEQ ID NO 177) was probed by assaying for IL-23 binding during systematic replacement of (deoxy guanosine) dG with deoxy inosine (dI). Since deoxy inosine lacks the exocyclic amine found in deoxy guanosine, a single amino to N7 hydrogen bond is removed from a potential G-quartet for each dG to dI substitution. As seen from the data, only significant substitutions lead to substantial decreases in affinity for IL-23 suggesting that the aptamer structure is robust. Additionally, the addition of phosphorothioate containing residues into the ARC1544 (SEQ ID NO 275) context was evaluated (comprising ARC1620 to ARC1626 (SEQ ID NOs 306-312). As can be seen in Table 30 the affinities of ARC1620-1626 (SEQ ID NOs 306-312) were in fact improved relative to ARC979 (SEQ ID NO 177). FIG. 17 depicts the binding curves for select ARC979 derivatives (ARC1624 and ARC1625) from the phase 3 optimization efforts, showing the remarkably improved binding affinities conferred by the inclusion of select phosphorothioate containing residues, compared to the parent molecule ARC979.

Phase 4 of optimization, comprised of ARC1755-1756 (SEQ ID NOs 313-314), involved only 2 sequences in an attempt to introduce more deoxy to 2′-O methyl substitutions and retain affinity. As can be seen with ARC1755 and 1756, these experiments were successful.

Unless noted otherwise, each of the sequences listed in Table 29 are in the 5′ to 3′ direction and may be derivatized with polyalkylene glycol (“PAG”) moieties.

TABLE 29

Sequence information Phase 1-4 ARC979
optimization

| | | | Sequence (5′ → 3′), (3T = |
|---|---|---|---|
| | | | inv dT), (T = dT), (s = |
| | | | phosphorothioate), (mN = |
| SEQ | | | 2′-O Methyl containing |
| ID | | | residue) (dI = deoxy |
| NO | ARC # | Description | inosine containing residue) |
| 224 | ARC1386 | ARC 979 | dAmCdAdGdGmCdAdAdGmUdAdAmUm |
| | | with | UdGdGdGdGdAdGmUdGmCdGdGdGmC |
| | | 3′-inv dT | dGdGdGdGmUdGmU-3T |
| 225 | ARC1427 | ARC979 opt | mAmCdAdGdGmCdAdAdGmUdAdAmUm |
| | | phase 1 | UdGdGdGdGdAdGmUdGmCdGdGdGmC |
| | | | dGdGdGdGmUdGmU-3T |
| 226 | ARC1428 | ARC979 opt | dAmCmAdGdGmCdAdAdGmUdAdAmUm |
| | | phase 1 | UdGdGdGdGdAdGmUdGmCdGdGdGmC |
| | | | dGdGdGdGmUdGmU-3T |
| 227 | ARC1429 | ARC979 opt | dAmCdAmGdGmCdAdAdGmUdAdAmUm |
| | | phase 1 | UdGdGdGdGdAdGmUdGmCdGdGdGmC |
| | | | dGdGdGdGmUdGmU-3T |
| 228 | ARC1430 | ARC979 opt | dAmCdAdGmGmCdAdAdGmUdAdAmUm |
| | | phase 1 | UdGdGdGdGdAdGmUdGmCdGdGdGmC |
| | | | dGdGdGdGmUdGmU-3T |
| 229 | ARC1431 | ARC979 opt | dAmCdAdGdGmCmAdAdGmUdAdAmUm |
| | | phase 1 | UdGdGdGdGdAdGmUdGmCdGdGdGmC |
| | | | dGdGdGdGmUdGmU-3T |
| 230 | ARC1432 | ARC979 opt | dAmCdAdGdGmCdAmAdGmUdAdAmUm |
| | | phase 1 | UdGdGdGdGdAdGmUdGmCdGdGdGmC |
| | | | dGdGdGdGmUdGmU-3T |
| 231 | ARC1433 | ARC979 opt | dAmCdAdGdGmCdAdAmGmUdAdAmUm |
| | | phase 1 | UdGdGdGdGdAdGmUdGmCdGdGdGmC |
| | | | dGdGdGdGmUdGmU-3T |
| 232 | ARC1434 | ARC979 opt | dAmCdAdGdGmCdAdAdGmUmAdAmUm |
| | | phase 1 | UdGdGdGdGdAdGmUdGmCdGdGdGmC |
| | | | dGdGdGdGmUdGmU-3T |
| 233 | ARC1435 | ARC979 opt | dAmCdAdGdGmCdAdAdGmUdAmAmUm |
| | | phase 1 | UdGdGdGdGdAdGmUdGmCdGdGdGmC |
| | | | dGdGdGdGmUdGmU-3T |
| 234 | ARC1436 | ARC979 opt | dAmCdAdGdGmCdAdAdGmUdAdAmUm |
| | | phase 1 | UmGdGdGdGdAdGmUdGmCdGdGdGmC |
| | | | dGdGdGdGmUdGmU-3T |
| 235 | ARC1437 | ARC979 opt | dAmCdAdGdGmCdAdAdGmUdAdAmUm |
| | | phase 1 | UdGmGdGdGdAdGmUdGmCdGdGdGmC |
| | | | dGdGdGdGmUdGmU-3T |
| 236 | ARC1438 | ARC979 opt | dAmCdAdGdGmCdAdAdGmUdAdAmUm |
| | | phase 1 | UdGdGmGdGdAdGmUdGmCdGdGdGmC |
| | | | dGdGdGdGmUdGmU-3T |
| 237 | ARC1439 | ARC979 opt | dAmCdAdGdGmCdAdAdGmUdAdAmUm |
| | | phase 1 | UdGdGdGmGdAdGmUdGmCdGdGdGmC |
| | | | dGdGdGdGmUdGmU-3T |
| 238 | ARC1440 | ARC979 opt | dAmCdAdGdGmCdAdAdGmUdAdAmUm |
| | | phase 1 | UdGdGdGdGmAdGmUdGmCdGdGdGmC |
| | | | dGdGdGdGmUdGmU-3T |
| 239 | ARC1441 | ARC979 opt | dAmCdAdGdGmCdAdAdGmUdAdAmUm |
| | | phase 1 | UdGdGdGdGdAmGmUdGmCdGdGdGmC |
| | | | dGdGdGdGmUdGmU-3T |
| 240 | ARC1442 | ARC979 opt | dAmCdAdGdGmCdAdAdGmUdAdAmUm |
| | | phase 1 | UdGdGdGdGdAdGmUmGmCdGdGdGmC |
| | | | dGdGdGdGmUdGmU-3T |
| 241 | ARC1443 | ARC979 opt | dAmCdAdGdGmCdAdAdGmUdAdAmUm |
| | | phase 1 | UdGdGdGdGdAdGmUdGmCmGdGdGmC |
| | | | dGdGdGdGmUdGmU-3T |
| 242 | ARC1444 | ARC979 opt | dAmCdAdGdGmCdAdAdGmUdAdAmUm |
| | | phase 1 | UdGdGdGdGdAdGmUdGmCdGmGdGmC |
| | | | dGdGdGdGmUdGmU-3T |
| 243 | ARC1445 | ARC979 opt | dAmCdAdGdGmCdAdAdGmUdAdAmUm |
| | | phase 1 | UdGdGdGdGdAdGmUdGmCdGdGmGmC |
| | | | dGdGdGdGmUdGmU-3T |
| 244 | ARC1446 | ARC979 opt | dAmCdAdGdGmCdAdAdGmUdAdAmUm |
| | | phase 1 | UdGdGdGdGdAdGmUdGmCdGdGdGmC |
| | | | mGdGdGdGmUdGmU-3T |
| 245 | ARC1447 | ARC979 opt | dAmCdAdGdGmCdAdAdGmUdAdAmUm |
| | | phase 1 | UdGdGdGdGdAdGmUdGmCdGdGdGmC |
| | | | dGmGdGdGmUdGmU-3T |
| 246 | ARC1448 | ARC979 opt | dAmCdAdGdGmCdAdAdGmUdAdAmUm |
| | | phase 1 | UdGdGdGdGdAdGmUdGmCdGdGdGmC |
| | | | dGdGmGdGmUdGmU-3T |
| 247 | ARC1449 | ARC979 opt | dAmCdAdGdGmCdAdAdGmUdAdAmUm |
| | | phase 1 | UdGdGdGdGdAdGmUdGmCdGdGdGmC |
| | | | dGdGdGmGmUdGmU-3T |
| 248 | ARC1450 | ARC979 opt | dAmCdAdGdGmCdAdAdGmUdAdAmUm |
| | | phase 1 | UdGdGdGdGdAdGmUdGmCdGdGdGmC |
| | | | dGdGdGdGmUmGmU-3T |
| 249 | ARC1451 | ARC979 opt | mAmCmAdGdGmCdAdAdGmUdAdAmUm |
| | | phase 1 | UdGdGdGdGdAdGmUdGmCdGdGdGmC |
| | | | dGdGdGdGmUmGmU3-T |
| 250 | ARC1452 | ARC979 opt | dAmCdAdGdGmCmAmAdGmUdAdAmUm |
| | | phase 1 | UmGdGdGdGdAdGmUdGmCdGdGdGmC |
| | | | dGdGdGdGmUdGmU-3T |
| 251 | ARC1453 | ARC979 opt | dAmCdA-sdGdGmCdAdAdGmUdAdAm |
| | | phase 1 | UmUdGdGdGdgdAdGmUdGmCdGdGdG |
| | | | mCdGdGdGdGmUdGmU3-T |
| 252 | ARC1454 | ARC979 opt | dAmCdAdG-sdGmCdAdAdGmUdAdAm |
| | | phase 1 | UmUdGdGdGdGdAdGmUdGmCdGdGdG |
| | | | mCdGdGdGdGmUdGmU3-T |
| 253 | ARC1455 | ARC979 opt | dAmCdAdGdG-s-mCdAdAdGmUdAdA |
| | | phase 1 | mUmUdGdGdGdGdAdGmUdGmCdGdGd |
| | | | GmCdGdGdGdGmUdGmU3-T |
| 254 | ARC1456 | ARC979 opt | dAmCdAdGdGmCdAdAdGmUdAdAmUm |
| | | phase 1 | UdG-s-dGdGdGdAdGmUdGmCdGdGd |
| | | | GmCdGdGdGdGmUdGmU-3T |
| 255 | ARC1457 | ARC979 opt | dAmCdAdGdGmCdAdAdGmUdAdAmUm |
| | | phase 1 | UdGdG-s-dGdGdAdGmUdGmCdGdGd |
| | | | GmCdGdGdGdGmUdGmU-3T |
| 256 | ARC1458 | ARC979 opt | dAmCdAdGdGmCdAdAdGmUdAdAmUm |
| | | phase 1 | UdGdGdG-s-dGdAdGmUdGmCdGdGd |
| | | | GmCdGdGdGdGmUdGmU-3T |
| 257 | ARC1459 | ARC979 opt | dAmCdAdGdGmCdAdAdGmUdAdAdUm |
| | | phase 1 | UdGdGdGdGdAdGmUdGmC-s-dGdGd |
| | | | GmCdGdGdGdGmUdGmU-3T |
| 258 | ARC1460 | ARC979 opt | dAmCdAdGdGmCdAdAdGmUdAdAmUm |
| | | phase 1 | UdGdGdGdGdAdGmUdGmCdG-s-dGd |
| | | | GmCdGdGdGdGmUdGmU-3T |
| 259 | ARC1461 | ARC979 opt | dAmCdAdGdGmCdAdAdGmUdAdAmUm |
| | | phase 1 | UdGdGdGdGdAdGmUdGmCdGdG-s-d |
| | | | GmCdGdGdGdGmUdGmU-3T |
| 260 | ARC1462 | ARC979 opt | dAmCdAdGdGmCdAdAdGmUdAdAmUm |
| | | phase 1 | UdGdGdGdGdAdGmUdGmCdGdGdGmC |
| | | | dGdG-s-dGdGmUdGmU-3T |
| 261 | ARC1463 | ARC979 opt | dAmCdAdGdGmCdAdAdGmUdAdAAnU |
| | | phase 1 | mUdGdGdGdGdAdGmUdGmCdGdGdGm |
| | | | CdGdGdG-s-dGmUdGmU-3T |
| 262 | ARC1464 | ARC979 opt | dAmCdAdGdGmCdAdAdGmUdAdAmUm |
| | | phase 1 | UdGdGdGdGdAdGmUdGmCdGdGdGmC |
| | | | dGdGdGdG-s-mUdGmU-3T |
| 263 | ARC1465 | ARC979 opt | dAmCdAdGdGmCdAdAdGmUdA-s-dA |
| | | phase 1 | mUmUdGdGdGdGdA-s-dG-s-mU-s- |
| | | | dG-s-mCdGdGdG-s-mCdGdGdGdGm |
| | | | UdGmU-3T |
| 264 | ARC1466 | ARC979 opt | dAmCdAPEGdGdGmCdAdAdGmUdAdA |
| | | phase 1 | mUmUdGdGdGdGdAdGmUdGmCdGdGd |
| | | | GmCdGdGdGdGPEGmUdGmU-3T |
| 265 | ARC1467 | ARC979 opt | mCmGmCdAPEGdGdGmCdAdAdGmUdA |
| | | phase 1 | dAmUmUdGdGdGdGdAdGmUdGmCdGd |
| | | | GdGmCdGdGdGdGPEGmUdGmCmG-3T |
| 266 | ARC1468 | ARC979 opt | dGdGmCdAdAdGmUdAdAmUmUdGdGd |
| | | phase 1 | GdGdAdGmUdGmCdGdGdGmCdGdGdG |
| | | | dG-3T |
| 267 | ARC1469 | ARC979 opt | dGdGmCmAmAdGmUdAdAmUmUmGdGd |
| | | phase 1 | GdGdAdGmUdGmCdGdGdGmCdGdGdG |
| | | | dG-3T |
| 268 | ARC1470 | ARC979 opt | dGdGmCdAdAdGmUdA-s-dAmUmUdG |
| | | phase 1 | dGdGdGdA-s-dG-s-mU-s-dG-s-m |
| | | | CdGdGdG-s-mCdGdGdGdG-3T |
| 269 | ARC1471 | ARC979 opt | dGdGmCmAmAdGmUdA-s-dAmUmUmG |
| | | phase 1 | dGdGdGdA-s-dG-s-mU-s-dG-s-m |
| | | | CdGdGdG-s-mCdGdGdGdG-3T |
| 270 | ARC1539 | ARC979 opt | mAmCdAdGdGmCdAdAdGmUdAdAmUm |
| | | phase 2 | UdGdGdGdGdAdGmUdGmCdGdGdGmC |
| | | | dGdGdGdGmUmGmU-3T |
| 271 | ARC1540 | ARC979 opt | dAmCdAdGdGmCdAmAmGmUmAdAmUm |
| | | phase 2 | UdGdGdGdGdAdGmUdGmCdGdGdGmC |
| | | | dGdGdGdGmUdGmU-3T |
| 272 | ARC1541 | ARC979 opt | dAmCdAdGdGmCdAdAdGmUdAdAmUm |
| | | phase 2 | UdGdGdGmGmAmGmUmGmCdGdGdGmC |
| | | | dGdGdGdGmUdGmU-3T |
| 273 | ARC1542 | ARC979 opt | dAmCdAdGdGmCdAdAdGmUdAdAmUm |
| | | phase 2 | UdGdGdGdGdAdGmUdGmCdGdGmGmC |
| | | | mGmGdGdGmUdGmU-3T |
| 274 | ARC1543 | ARC979 opt | mAmCdAdGdGmCdAmAmGmUmAdAmUm |
| | | phase 2 | UdGdGdGmGmAmGmUmGmCdGdGmGmC |
| | | | mGmGdGdGmUmGmU-3T |
| 275 | ARC1544 | ARC979 opt | dAmCdAdGdGmCdAdAdGmUdAdAmUm |
| | | phase 2 | UdGmGmGdGdAdGmUdGmCmGmGdGmC |
| | | | dGdGmGmGmUdGmU-3T |
| 276 | ARC1545 | ARC979 opt | mAmCdAdGdGmCdAmAmGmUmAdAmUm |
| | | phase 2 | UdGmGmGmGmAmGmUmGmCmGmGmGmC |
| | | | mGmGmGmGmUmGmU-3T |
| 277 | ARC1591 | ARC979 opt | dAmCdAdIdGmCdAdAdGmUdAdAmUm |
| | | phase 3 | UdGdGdGdGdAdGmUdGmCdGdGdGmC |
| | | | dGdGdGdGmUdGmU-3T |
| 278 | ARC1592 | ARC979 opt | dAmCdAdGdImCdAdAdGmUdAdAmUm |
| | | phase 3 | UdGdGdGdGdAdGmUdGmCdGdGdGmC |
| | | | dGdGdGdGmUdGmU-3T |
| 279 | ARC1593 | ARC979 opt | dAmCdAdIdImCdAdAdGmUdAdAmUm |
| | | phase 3 | UdGdGdGdGdAdGmUdGmCdGdGdGmC |
| | | | dGdGdGdGmUdGmU-3T |
| 280 | ARC1594 | ARC979 opt | dAmCdAdGdGmCdAdAdImUdAdAmUm |
| | | phase 3 | UdGdGdGdGdAdGmUdGmCdGdGdGmC |
| | | | dGdGdGdGmUdGmU-3T |
| 281 | ARC1595 | ARC979 opt | dAmCdAdGdGmCdAdAdGmUdAdAmUm |
| | | phase 3 | UdIdGdGdGdAdGmUdGmCdGdGdGmC |
| | | | dGdGdGdGmUdGmU-3T |
| 282 | ARC1596 | ARC979 opt | dAmCdAdGdGmCdAdAdGmUdAdAmUm |
| | | phase 3 | UdGdIdGdGdAdGmUdGmCdGdGdGmC |
| | | | dGdGdGdGmUdGmU-3T |
| 283 | ARC1597 | ARC979 opt | dAmCdAdGdGmCdAdAdGmUdAdAmUm |
| | | phase 3 | UdGdGdIdGdAdGmUdGmCdGdGdGmC |
| | | | dGdGdGdGmUdGmU-3T |
| 284 | ARC1598 | ARC979 opt | dAmCdAdGdGmCdAdAdGmUdAdAmUm |
| | | phase 3 | UdGdGdGdIdAdGmUdGmCdGdGdGmC |
| | | | dGdGdGdGmUdGmU-3T |
| 285 | ARC1599 | ARC979 opt | dAmCdAdGdGmCdAdAdGmUdAdAmUm |
| | | phase 3 | UdIdIdGdGdAdGmUdGmCdGdGdGmC |
| | | | dGdGdGdGmUdGmU-3T |
| 286 | ARC1600 | ARC979 opt | dAmCdAdGdGmCdAdAdGmUdAdAmUm |
| | | phase 3 | UdGdIdIdGdAdGmUdGmCdGdGdGmC |
| | | | dGdGdGdGmUdGmU-3T |
| 287 | ARC1601 | ARC979 opt | dAmCdAdGdGmCdAdAdGmUdAdAmUm |
| | | phase 3 | UdGdGdIdIdAdGmUdGmCdGdGdGmC |
| | | | dGdGdGdGmUdGmU-3T |
| 288 | ARC1602 | ARC979 opt | dAmCdAdGdGmCdAdAdGmUdAdAmUm |
| | | phase 3 | UdIdIdIdIAdGmUdGmCdGdGdGmCd |
| | | | GdGdGdGmUdGmU-3T |
| 289 | ARC1603 | ARC979 opt | dAmCdAdGdGmCdAdAdGmUdAdAmUm |
| | | phase 3 | UdGdGdGdGdAdImUdGmCdGdGdGmC |
| | | | dGdGdGdGmUdGmU-3T |
| 290 | ARC1604 | ARC979 opt | dAmCdAdGdGmCdAdAdGmUdAdAmUm |
| | | phase 3 | UdGdGdGdGdAdGmUdImCdGdGdGmC |
| | | | dGdGdGdGmUdGmU-3T |
| 291 | ARC1605 | ARC979 opt | dAmCdAdGdGmCdAdAdGmUdAdAmUm |
| | | phase 3 | UdGdGdGdGdAdGmUdGmCdIdGdGmC |
| | | | dGdGdGdGmUdGmU-3T |
| 292 | ARC1606 | ARC979 opt | dAmCdAdGdGmCdAdAdGmUdAdAmUm |
| | | phase 3 | UdGdGdGdGdAdGmUdGmCdGdIdGmC |
| | | | dGdGdGdGmUdGmU-3T |
| 293 | ARC1607 | ARC979 opt | dAmCdAdGdGmCdAdAdGmUdAdAmUm |
| | | phase 3 | UdGdGdGdGdAdGmUdGmCdGdGdImC |
| | | | dGdGdGdGmUdGmU-3T |
| 294 | ARC1608 | ARC979 opt | dAmCdAdGdGmCdAdAdGmUdAdAmUm |
| | | phase 3 | UdGdGdGdGdAdGmUdGmCdIdIdGmC |
| | | | dGdGdGdGmUdGmU-3T |
| 295 | ARC1609 | ARC979 opt | dAmCdAdGdGmCdAdAdGmUdAdAmUm |
| | | phase 3 | UdGdGdGdGdAdGmUdGmCdGdIdImC |
| | | | dGdGdGdGmUdGmU-3T |
| 296 | ARC1610 | ARC979 opt | dAmCdAdGdGmCdAdAdGmUdAdAmUm |
| | | phase 3 | UdGdGdGdGdAdGmUdGmCdIdIdImC |
| | | | dGdGdGdGmUdGmU-3T |
| 297 | ARC1611 | ARC979 opt | dAmCdAdGdGmCdAdAdGmUdAdAAmU |
| | | phase 3 | mUdGdGdGdGdAdGmUdGmCdGdGdGm |
| | | | CdIdGdGdGmUdGmU-3T |
| 298 | ARC1612 | ARC979 opt | dAmCdAdGdGmCdAdAdGmUdAdAmUm |
| | | phase 3 | UdGdGdGdGdAdGmUdGmCdGdGdGmC |
| | | | dGdIdGdGmUdGmU-3T |
| 299 | ARC1613 | ARC979 opt | dAmCdAdGdGmCdAdAdGmUdAdAmUm |
| | | phase 3 | UdGdGdGdGdAdGmUdGmCdGdGdGmC |
| | | | dGdGdIdGmUdGmU-3T |
| 300 | ARC1614 | ARC979 opt | dAmCdAdGdGmCdAdAdGmUdAdAmUm |
| | | phase 3 | UdGdGdGdGdAdGmUdGmCdGdGdGmC |
| | | | dGdGdGdImUdGmU-3T |
| 301 | ARC1615 | ARC979 opt | dAmCdAdGdGmCdAdAdGmUdAdAmUm |
| | | phase 3 | UdGdGdGdGdAdGmUdGmCdGdGdGmC |
| | | | dIdIdGdGmUdGmU-3T |
| 302 | ARC1616 | ARC979 opt | dAmCdAdGdGmCdAdAdGmUdAdAmUm |
| | | phase 3 | UdGdGdGdGdAdGmUdGmCdGdGdGmC |
| | | | dGdIdIdGmUdGmU-3T |
| 303 | ARC1617 | ARC979 opt | dAmCdAdGdGmCdAdAdGmUdAdAmUm |
| | | phase 3 | UdGdGdGdGdAdGmUdGmCdGdGdGmC |
| | | | dGdGdIdImUdGmU-3T |
| 304 | ARC1618 | ARC979 opt | dAmCdAdGdGmCdAdAdGmUdAdAmUm |
| | | phase 3 | UdGdGdGdGdAdGmUdGmCdGdGdGmC |
| | | | dIdIdIdImUdGmU-3T |
| 305 | ARC1619 | ARC979 opt | dAmCdAdGdGmCdAdAdGmUdAdAmUm |
| | | phase 3 | UdGdGdGdGdAdGmUdGmCdGdGdGmC |
| | | | dGdGdGdGmUdImU-3T |
| 306 | ARC1620 | ARC979 opt | dAmC-s-dAdGdGmCdAdAdGmUdAdA |
| | | phase 3 | AmUmUdGmGmGdGdAdGmUdGmCmGmG |
| | | | dGmCdGdGmGmGmUdGmU-3T |
| 307 | ARC1621 | ARC979 opt | dAmCdA-s-dG-s-dGmCdAdAdGmUd |
| | | phase 3 | AdAmUmUdGmGmGdGdAdGmUdGmCmG |
| | | | mGdGmCdGdGmGmGmUdGmU-3T |
| 308 | ARC1622 | ARC979 opt | dAmCdAdGdGmC-s-dA-s-dA-s-dG |
| | | phase 3 | mU-s-dA-s-dAmUmU-s-dGmGmGdG |
| | | | dAdGmUdGmCmGmGdGmCdGdGmGmGm |
| | | | UdGmU-3T |
| 309 | ARC1623 | ARC979 opt | dAmCdAdGdGmCdAdAdGmUdAdAmUm |
| | | phase 3 | UdGmGmG-s-dG-s-dA-s-dGmU-s- |
| | | | dGmCmGmGdGmCdGdGmGmGmUdGmU- |
| | | | 3T |
| 310 | ARC1624 | ARC979 opt | dAmCdAdGdGmCdAdAdGmUdAdAmUm |
| | | phase 3 | UdGmGmGdGdAdGmUdGmCmGmG-s-d |
| | | | GmC-s-dG-s-dGmGmGmUdGmU-3T |
| 311 | ARC1625 | ARC979 opt | dAmCdAdGdGmCdAdAdGmUdAdAmUm |
| | | phase 3 | UdGmGmGdGdAdGmUdGmCmGmGdGmC |
| | | | dGdGmGmGmU-s-dGmU-3T |
| 312 | ARC1626 | ARC979 opt | dAmC-s-dA-s-dG-s-dGmC-s-dA- |
| | | phase 3 | s-dA-s-dGmU-s-dA-s-dAmUmU- |
| | | | s-dGmGmG-s-dG-s-dA-s-dGmU- |
| | | | s-dGmCmGmG-s-dGmC-s-dG-s-dG |
| | | | mGmGmU-s-dGmU-3T |
| 313 | ARC1755 | ARC979 opt | mAmC-s-dAdGdGmC-s-dAmAmGmUm |
| | | phase 4 | A-s-dAmUmU-s-dGmGmGmGmAmGmU |
| | | | mGmCmGmGmGmCmGmGmGmGmUmGmU- |
| | | | 3T |
| 314 | ARC1756 | ARC979 opt | mAmC-s-dAdGdGmC-s-dAmAmGmUm |
| | | phase 4 | A-s-dAmUmU-s-dGmGmG-s-dG-s- |
| | | | dA-s-dGmU-s-dGmCmGmGmGmCmGm |
| | | | GmGmGmUmGmU-3T |

TABLE 30
Binding Characterization

| | | | | % binding |
|---|---|---|---|---|
| | | | | at 100 nM |
| | | | | (through |
| | | | | ARC1619) |
| | | | | or at 10 nM |
| SEQ ID NO | ARC # | Description | K_{D} (nM) | (ARC1620-1756) |
| 224 | ARC1386 | ARC 979 | 1 | 69.9 |
| | | with 3′-inv | | |
| | | dT | | |
| 225 | ARC1427 | ARC979 opt | 3.0 | 49.4 |
| | | phase 1 | | |
| 226 | ARC1428 | ARC979 opt | 1.8 | 57.8 |
| | | phase 1 | | |
| 227 | ARC1429 | ARC979 opt | 29.5 | 48.4 |
| | | phase 1 | | |
| 228 | ARC1430 | ARC979 opt | 14.2 | 51.6 |
| | | phase 1 | | |
| 229 | ARC1431 | ARC979 opt | 10.0 | 56.3 |
| | | phase 1 | | |
| 230 | ARC1432 | ARC979 opt | 3.8 | 57.9 |
| | | phase 1 | | |
| 231 | ARC1433 | ARC979 opt | 2.8 | 55.2 |
| | | phase 1 | | |
| 232 | ARC1434 | ARC979 opt | 3.0 | 52.9 |
| | | phase 1 | | |
| 233 | ARC1435 | ARC979 opt | 9.8 | 51.2 |
| | | phase 1 | | |
| 234 | ARC1436 | ARC979 opt | 15.1 | 46.9 |
| | | phase 1 | | |
| 235 | ARC1437 | ARC979 opt | 3.9 | 43.1 |
| | | phase 1 | | |
| 236 | ARC1438 | ARC979 opt | 6.0 | 36.7 |
| | | phase 1 | | |
| 237 | ARC1439 | ARC979 opt | 4.8 | 43.5 |
| | | phase 1 | | |
| 238 | ARC1440 | ARC979 opt | 6.7 | 54.9 |
| | | phase 1 | | |
| 239 | ARC1441 | ARC979 opt | 2.7 | 49.8 |
| | | phase 1 | | |
| 240 | ARC1442 | ARC979 opt | 2.8 | 60.5 |
| | | phase 1 | | |
| 241 | ARC1443 | ARC979 opt | 2.0 | 52.8 |
| | | phase 1 | | |
| 242 | ARC1444 | ARC979 opt | 4.4 | 58.1 |
| | | phase 1 | | |
| 243 | ARC1445 | ARC979 opt | 2.8 | 56.3 |
| | | phase 1 | | |
| 244 | ARC1446 | ARC979 opt | 2.1 | 55.0 |
| | | phase 1 | | |
| 245 | ARC1447 | ARC979 opt | 2.5 | 56.5 |
| | | phase 1 | | |
| 246 | ARC1448 | ARC979 opt | 2.3 | 59.5 |
| | | phase 1 | | |
| 247 | ARC1449 | ARC979 opt | 2.6 | 48.4 |
| | | phase 1 | | |
| 248 | ARC1450 | ARC979 opt | 2.6 | 46.5 |
| | | phase 1 | | |
| 249 | ARC1451 | ARC979 opt | 10.2 | 46.1 |
| | | phase 1 | | |
| 250 | ARC1452 | ARC979 opt | 18.9 | 56.9 |
| | | phase 1 | | |
| 251 | ARC1453 | ARC979 opt | 4.4 | 65.0 |
| | | phase 1 | | |
| 252 | ARC1454 | ARC979 opt | 2.7 | 61.6 |
| | | phase 1 | | |
| 253 | ARC1455 | ARC979 opt | 1.6 | 56.6 |
| | | phase 1 | | |
| 254 | ARC1456 | ARC979 opt | 3.2 | 55.5 |
| | | phase 1 | | |
| 255 | ARC1457 | ARC979 opt | 3.0 | 56.1 |
| | | phase 1 | | |
| 256 | ARC1458 | ARC979 opt | 2.9 | 49.6 |
| | | phase 1 | | |
| 257 | ARC1459 | ARC979 opt | 4.0 | 50.7 |
| | | phase 1 | | |
| 258 | ARC1460 | ARC979 opt | 5.8 | 46.1 |
| | | phase 1 | | |
| 259 | ARC1461 | ARC979 opt | 3.7 | 47.3 |
| | | phase 1 | | |
| 260 | ARC1462 | ARC979 opt | 1.7 | 53.4 |
| | | phase 1 | | |
| 261 | ARC1463 | ARC979 opt | 3.6 | 53.5 |
| | | phase 1 | | |
| 262 | ARC1464 | ARC979 opt | 2.4 | 54.6 |
| | | phase 1 | | |
| 263 | ARC1465 | ARC979 opt | 1.3 | 57.0 |
| | | phase 1 | | |
| 264 | ARC1466 | ARC979 opt | 1.9 | 38.7 |
| | | phase 1 | | |
| 265 | ARC1467 | ARC979 opt | 1.7 | 57.0 |
| | | phase 1 | | |
| 266 | ARC1468 | ARC979 opt | 10.0 | 49.8 |
| | | phase 1 | | |
| 267 | ARC1469 | ARC979 opt | 49.8 | 59.8 |
| | | phase 1 | | |
| 268 | ARC1470 | ARC979 opt | 8.6 | 61.0 |
| | | phase 1 | | |
| 269 | ARC1471 | ARC979 opt | 23.5 | 62.9 |
| | | phase 1 | | |
| 270 | ARC1539 | ARC979 opt | 6.6 | 43.8 |
| | | phase 2 | | |
| 271 | ARC1540 | ARC979 opt | 7.5 | 50.3 |
| | | phase 2 | | |
| 272 | ARC1541 | ARC979 opt | 3.9 | 57.0 |
| | | phase 2 | | |
| 273 | ARC1542 | ARC979 opt | 1.2 | 57.6 |
| | | phase 2 | | |
| 274 | ARC1543 | ARC979 opt | 5.9 | 40.9 |
| | | phase 2 | | |
| 275 | ARC1544 | ARC979 opt | 0.9 | 58.6 |
| | | phase 2 | | |
| 276 | ARC1545 | ARC979 opt | 0.4 & 62.0 | 17.4 & 20.9 |
| | | phase 2 | (the binding | |
| | | | curve was | |
| | | | strongly | |
| | | | biphasic) | |
| 277 | ARC1591 | ARC979 opt | | 54.8 |
| | | phase 3 | | |
| 278 | ARC1592 | ARC979 opt | 8.1 | 54.4 |
| | | phase 3 | | |
| 279 | ARC1593 | ARC979 opt | 13.8 | 51.0 |
| | | phase 3 | | |
| 280 | ARC1594 | ARC979 opt | 4.2 | 60.1 |
| | | phase 3 | | |
| 281 | ARC1595 | ARC979 opt | 5.4 | 53.9 |
| | | phase 3 | | |
| 282 | ARC1596 | ARC979 opt | 11.1 | 59.0 |
| | | phase 3 | | |
| 283 | ARC1597 | ARC979 opt | 11.2 | 61.3 |
| | | phase 3 | | |
| 284 | ARC1598 | ARC979 opt | 4.7 | 61.0 |
| | | phase 3 | | |
| 285 | ARC1599 | ARC979 opt | 7.2 | 57.7 |
| | | phase 3 | | |
| 286 | ARC1600 | ARC979 opt | 15.6 | 61.3 |
| | | phase 3 | | |
| 287 | ARC1601 | ARC979 opt | 4.4 | 58.6 |
| | | phase 3 | | |
| 288 | ARC1602 | ARC979 opt | 40.8 | 64.4 |
| | | phase 3 | | |
| 289 | ARC1603 | ARC979 opt | 1.6 | 64.2 |
| | | phase 3 | | |
| 290 | ARC1604 | ARC979 opt | 2.1 | 50.2 |
| | | phase 3 | | |
| 291 | ARC1605 | ARC979 opt | 7.5 | 56.8 |
| | | phase 3 | | |
| 292 | ARC1606 | ARC979 opt | 5.0 | 60.3 |
| | | phase 3 | | |
| 293 | ARC1607 | ARC979 opt | 3.3 | 61.5 |
| | | phase 3 | | |
| 294 | ARC1608 | ARC979 opt | 9.7 | 61.1 |
| | | phase 3 | | |
| 295 | ARC1609 | ARC979 opt | 4.7 | 60.5 |
| | | phase 3 | | |
| 296 | ARC1610 | ARC979 opt | 5.2 | 60.4 |
| | | phase 3 | | |
| 297 | ARC1611 | ARC979 opt | 1.7 | 62.1 |
| | | phase 3 | | |
| 298 | ARC1612 | ARC979 opt | 1.9 | 60.9 |
| | | phase 3 | | |
| 299 | ARC1613 | ARC979 opt | 2.3 | 58.4 |
| | | phase 3 | | |
| 300 | ARC1614 | ARC979 opt | 1.7 | 60.5 |
| | | phase 3 | | |
| 301 | ARC1615 | ARC979 opt | 5.8 | 55.2 |
| | | phase 3 | | |
| 302 | ARC1616 | ARC979 opt | 6.1 | 59.5 |
| | | phase 3 | | |
| 303 | ARC1617 | ARC979 opt | 4.1 | 61.9 |
| | | phase 3 | | |
| 304 | ARC1618 | ARC979 opt | 34.0 | 67.0 |
| | | phase 3 | | |
| 305 | ARC1619 | ARC979 opt | 2.8 | 52.1 |
| | | phase 3 | | |
| 306 | ARC1620 | ARC979 opt | 0.4 | 68.0 |
| | | phase 3 | | |
| 307 | ARC1621 | ARC979 opt | 0.5 | 64.6 |
| | | phase 3 | | |
| 308 | ARC1622 | ARC979 opt | 0.3 | 66.0 |
| | | phase 3 | | |
| 309 | ARC1623 | ARC979 opt | 0.2 | 68.7 |
| | | phase 3 | | |
| 310 | ARC1624 | ARC979 opt | 0.4 | 68.0 |
| | | phase 3 | | |
| 311 | ARC1625 | ARC979 opt | 0.4 | 75.0 |
| | | phase 3 | | |
| 312 | ARC1626 | ARC979 opt | 0.1 | 79.2 |
| | | phase 3 | | |
| 313 | ARC1755 | ARC979 opt | 0.8 | 31 |
| | | phase 4 | | |
| 314 | ARC1756 | ARC979 opt | 0.5 | 56 |
| | | phase 4 | | |

*30 min RT incubation for K_{D} determination
*1X Dulbecco's PBS (with Ca⁺⁺ and Mg⁺⁺) + 0.1 mg/mL BSA reaction buffer

### Example 2C

### Plasma Stability of Anti-IL-23 Aptamers

A subset of the aptamers identified during the optimization process was assayed for nuclease stability in human plasma. Plasma nuclease degradation was measured using denaturing polyacrylamide gel electrophoresis as described below. Briefly, for plasma stability determination, chemically synthesized aptamers were purified using denaturing polyacrylamide gel electrophoresis, 5′ end labeled with γ-³²P ATP and then gel purified again. Trace ³²P labeled aptamer was incubated in the presence of 100 nM unlabeled aptamer in 95% human plasma in a 200 microliter binding reaction. The reaction for the time zero point was made separately with the same components except that the plasma was replaced with PBS to ensure that the amount of radioactivity loaded on gels was consistent across the experiment. Reactions were incubated at 37° C. in a thermocycler for the 1, 3, 10, 30 and 100 hours. At each time point, 20 microliters of the reaction was removed, combined with 200 microliters of formamide loading dye and flash frozen in liquid nitrogen and stored at −20° C. After the last time point was taken, frozen samples were thawed and 20 microliters was removed from each time point. SDS was then added to the small samples to a final concentration of 0.1%. The samples were then incubated at 90° C. for 10-15 minutes and loaded directly onto a 15% denaturing PAGE gel and run at 12 W for 35 minutes. Radioactivity on the gels was quantified using a Storm 860 Phosphorimager system (Amersham Biosciences, Piscataway, N.J.). The percentage of full length aptamer at each time point was determined by quantifying the full length aptamer band and dividing by the total counts in the lane. The fraction of full length aptamer at each time-point was then normalized to the percentage full length aptamer of the 0 hour time-point. The fraction of full length aptamer as a function of time was fit to the equation:

m1*ê(−m2*m0)
where m¹ is the maximum % full length aptamer (m1=100); and m2 is the rate of degradation.
   The half-life of the aptamer (T_{1/2}) is equal to the (ln 2)/m2.

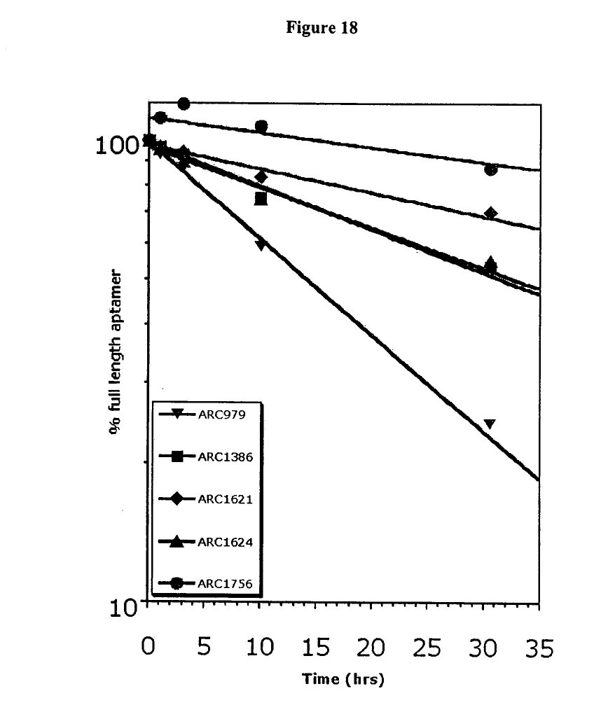

Sample data is shown in FIG. 18 and the results for the aptamers tested are summarized in Table 31.

TABLE 31
plasma stability

| | | | ~T½ in |
|---|---|---|---|
| | | | human |
| SEQ ID NO | ARC # | Description | plasma (hrs) |
| 177 | ARC979 | | 14 |
| 224 | ARC1386 | ARC 979 | 33 |
| | | with 3′-inv | |
| | | dT | |
| 307 | ARC1621 | ARC979 opt | 59 |
| | | phase 3 | |
| 308 | ARC1622 | ARC979 opt | 54 |
| | | phase 3 | |
| 309 | ARC1623 | ARC979 opt | 45 |
| | | phase 3 | |
| 310 | ARC1624 | ARC979 opt | 35 |
| | | phase 3 | |
| 311 | ARC1625 | ARC979 opt | 31 |
| | | phase 3 | |
| 312 | ARC1626 | ARC979 opt | 113 |
| | | phase 3 | |
| 313 | ARC1755 | ARC979 opt | 83 |
| | | phase 4 | |
| 314 | ARC1756 | ARC979 opt | 96 |
| | | phase 4 | |

### Example 3

### Functional Cell Assays

Cell-Based Assay and Minimization of Active rRfY IL-23 Aptamers

IL-23 plays a role in JAK/STAT signal transduction and phosphorylates STAT 1, 3, 4, and 5. To test whether IL-23 aptamers showed cell-based activity, signal transduction was assayed in the lysates of peripheral blood mononuclear cells (PBMCs) grown in media containing PHA (Phytohemagglutinin), or PHA Blasts. More specifically, the cell-based assay determined whether IL-23 aptamers could inhibit IL-23 induced STAT-3 phosphorylation in PHA Blasts.

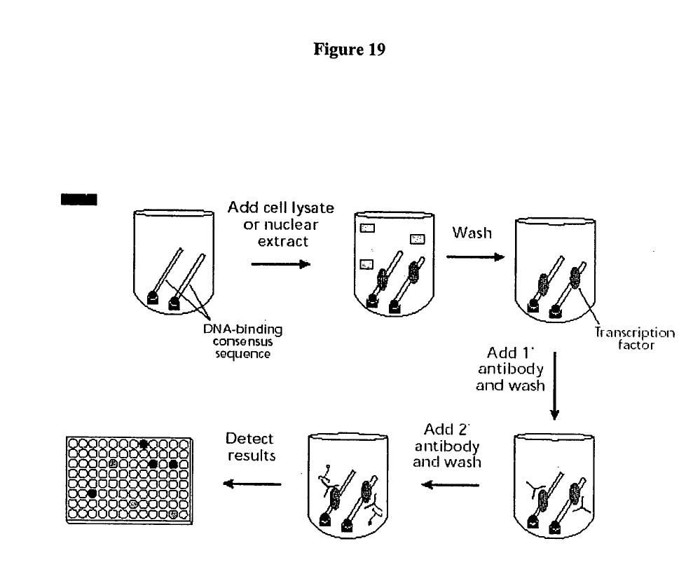

In essence, lysates of IL-23 treated cells will contain more activated STAT3 than quiescent or aptamer blocked cells. Inhibition of IL-23-induced STAT3 phosphorylation was measured by two methods: by western blot, using an anti-phospho-STAT3 Antibody (Tyr705) (Cell Signaling, Beverly, Mass.); and by TransAM™ Assay (Active Motif, Carlsbad, Calif.). The TransAM™ assay kit provides a 96 well plate on which an oligonucleotide containing the STAT consensus binding site (5′TTCCCGGAA-3′) is immobilized. An anti-STAT3 antibody that recognizes an epitope on STAT3 that is only accessible when STAT3 is activated is used in conjunction with an HRP-conjugated secondary antibody to give a colorimetric readout that can be quantified by spectrophotometry. (See FIG. 19).

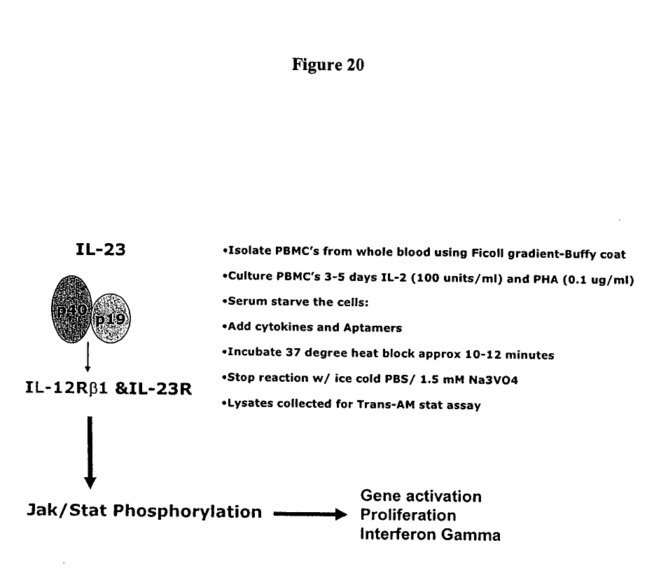

In summary, the cell-based assay was conducted by isolating the peripheral blood mononuclear cells (PBMCs) from whole blood using a Histopaque gradient (Sigma, St. Louis, Mo.). The PBMCs were cultured for 3 to 5 days at 37° C./5% CO₂ in Peripheral Blood Medium (Sigma) which contains PHA, supplemented with IL-2 (100 units/mL) (R&D Systems, Minneapolis, Minn.), to generate PHA Blasts. To test IL-23 aptamers, the PHA Blasts were washed twice with 1×PBS, then serum starved for four hours in RPMI, 0.20% FBS. After serum starvation, approximately 2 million cells were aliquotted into appropriately labeled eppendorf tubes. hIL-23 at a final constant concentration of 3 ng/mL (R&D Systems, Minneapolis, Minn.) was combined with a dilution series of various IL-23 aptamers as described in Example 1, and the cytokine/aptamer mixture was added to the aliquotted cells in a final volume of 100 μl and incubated at 37° C. for 10-12 minutes. The incubation reaction was stopped by adding 1 mL of ice-cold PBS with 1.5 mM Na₃VO₄. Cell lysates were made using the lysis buffer provided by the TransAM™ STAT 3 assay following the manufacturer's instructions. FIG. 20 depicts a flow summary of the protocol used for the cell based assay.

Parent aptamer and minimized IL-23 aptamers from the various selections with 2′-F pyrimidines-containing pools (rRfY), ribo/2′O-Me containing pools (rRmY), deoxy/2′O-Me containing pools (dRmY), and optimized dRmY aptamers were tested using the TransAM™ method.

### Example 3A

### Cell Based Assay Results for Parent and Minimized Clones from rRfY Selections

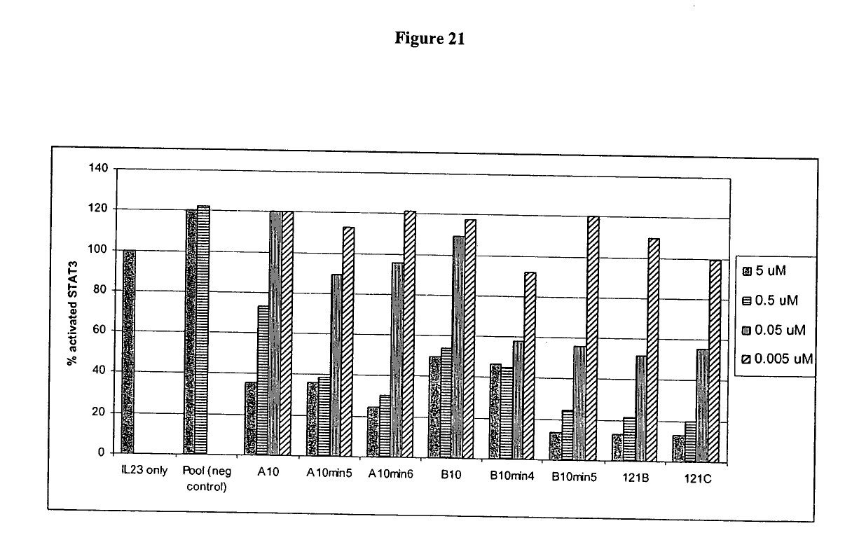

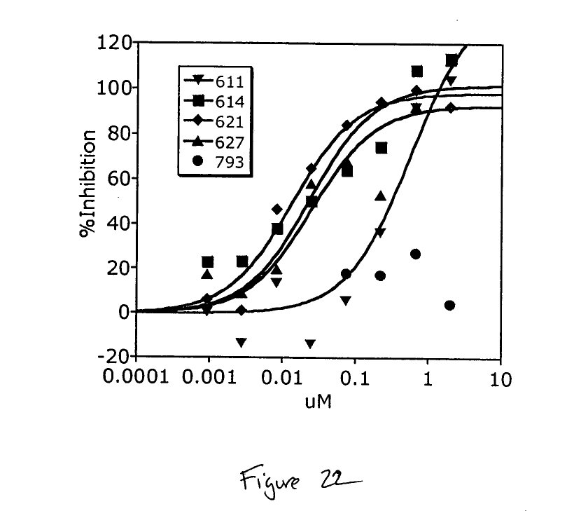

Full length clones from the rRfY selection described in Example 1A, and select minimized rRfY clones that were described in Example 2A.1, were tested using the TransAM™ STAT3 activation assay. Table 34 summarizes the cell based assay data for IL-23 full length aptamers generated from the rRfY selections described in Example 1A. Table 35 summarizes the activity data of the rRfY minimized clones, described in Example 2A.1, each compared to the activity of their respective parent (full length) clone. The minimized rRfY clones F11min2 (SEQ ID NO 147), A10min5 (SEQ ID NO 139), A10min6 (SEQ ID NO 140), B10 min4 (SEQ ID NO 144), B10min5 (SEQ ID NO 145), Type1.4 (SEQ ID NO 151) and Type1.5 (SEQ ID NO 152) each outperformed their respective parent clones (see FIG. 21), in addition to all of the full length rRfY clones when tested in the TransAM™ STAT3 activation assay.

TABLE 34
Cell Based Assay Results: Summary of rRfY Clones Tested

| SEQ | Clone | | | Western | | |
|---|---|---|---|---|---|---|
| ID NO | Name | selection | Blot | TransAM | TransAM IC₅₀ | |
| 27 | AMX86- | R8 h-IL-23 | Yes | Yes | 3 | μM |
| | C5 | | | | | |
| 13 | AMX86- | R8 h-IL-23 | Yes | Yes | >5 | μM |
| | D5 | | | | | |
| 16 | AMX86- | R8 h-IL-23 | Yes | Yes | >5 | μM |
| | D6 | | | | | |
| 24 | AMX86- | R8 h-IL-23 | Yes | No | | |
| | E6 | | | | | |
| 22 | AMX86- | R8 h-IL-23 | Yes | No | | |
| | F6 | | | | | |
| 18 | AMX86- | R8 h-IL-23 | Yes | No | | |
| | A7 | | | | | |
| 25 | AMX86- | R8 h-IL-23 | Yes | No | | |
| | H7 | | | | | |
| 35 | AMX86- | R8 X-IL-23 | Yes | No | | |
| | B9 | | | | | |
| 32 | AMX86- | R8 X-IL-23 | Yes | No | | |
| | C9 | | | | | |
| 33 | AMX86- | R8 X-IL-23 | Yes | No | | |
| | G9 | | | | | |
| 39 | AMX86- | R8 X-IL-23 | Yes | Yes | 250 | nM |
| | H9 | | | | | |
| 28 | AMX86- | R8 X-IL-23 | Yes | Yes | 800 | nM |
| | B10 | | | | | |
| 36 | AMX86- | R8 X-IL-23 | Yes | Yes | ~2 | μM |
| | G10 | | | | | |
| 37 | AMX86- | R8 X-IL-23 | Yes | No | | |
| | A11 | | | | | |
| 30 | AMX86- | R8 X-IL-23 | Yes | No | | |
| | D11 | | | | | |
| 43 | AMX84- | R10 PN-IL-23 | Yes | Yes | 400 | nM |
| | A10 | | | | | |
| 44 | AMX84- | R10 PN-IL-23 | Yes | Yes | >1 | μM |
| | B10 | | | | | |
| 45 | AMX84- | R10 PN-IL-23 | Yes | Yes | >5 | μM |
| | A11 | | | | | |
| 46 | AMX84- | R10 PN-IL-23 | Yes | Yes | 250 | nM |
| | F11 | | | | | |
| 47 | AMX84- | R10 PN-IL-23 | Yes | Yes | >1 | μM |
| | E12 | | | | | |
| 48 | AMX84- | R10 PN-IL-23 | No | Yes | 250 | nM |
| | C10 | | | | | |
| 49 | AMX84- | R10 PN-IL-23 | No | Yes | 800 | nM |
| | C11 | | | | | |
| 50 | AMX84- | R10 PN-IL-23 | No | Yes | 250 | nM |
| | G11 | | | | | |
| 51 | ARX83- | R12 PN-IL23 | No | Yes | >5 | μM |
| | plate1- | | | | | |
| | H1 | | | | | |
| 52 | AMX91- | R10 PN-IL-23 | No | Yes | 5 | μM |
| | F11 | | | | | |
| 53 | AMX91- | R10 PN-IL-23 | No | Yes | 2 | μM |
| | G1 | | | | | |
| 54 | AMX91- | R10 PN-IL-23 | No | Yes | >5 | μM |
| | E3 | | | | | |
| 55 | AMX91- | R10 PN-IL-23 | No | Yes | 50 | nM |
| | H3 | | | | | |
| 64 | AMX91- | R12 PN-IL23 | No | Yes | 3 | μM |
| | G11 | | | | | |
| 65 | AMX91- | R12 PN-IL23 | No | Yes | 50 | nM |
| | C12 | | | | | |
| 66 | AMX91- | R12 PN-IL23 | No | Yes | 350 | nM |
| | H12 | | | | | |
| 56 | AMX91- | R10 PN-IL-23 | No | Yes | 1 | μM |
| | B5 | | | | | |
| 57 | AMX91- | R10 PN-IL-23 | No | Yes | 3 | μM |
| | A6 | | | | | |
| 58 | AMX91- | R12 PN-IL23 | No | Yes | 150 | nM |
| | G7 | | | | | |
| 59 | AMX91- | R12 PN-IL23 | No | Yes | 50 | nM |
| | H7 | | | | | |
| 60 | AMX91- | R12 PN-IL23 | No | Yes | 450 | nM |
| | B8 | | | | | |
| 61 | AMX91- | R12 PN-IL23 | No | Yes | 3 | μM |
| | H8 | | | | | |
| 62 | AMX91- | R12 PN-IL23 | No | Yes | 50 | nM |
| | G9 | | | | | |
| 63 | AMX91- | R12 PN-IL23 | No | Yes | 150 | nM |
| | D9 | | | | | |

TABLE 35
IL-23 2′F rRfY Minimized aptamer binding compared to parent aptamers.

| SEQ | | | | | | | |
|---|---|---|---|---|---|---|---|
| ID | Clone | | | | IC₅₀ | IC₅₀ Full | |
| NO | Name | Selection | W.Blot | TransAM | minimer | Length | |
| 147 | F11min | R10 PN- | No | Yes | 25 nM | 250 | Nm |
| | 2 | IL-23 | | | | | |
| 139 | A10min | R10 PN- | No | Yes | 300 nM | 1 | μM |
| | 5 | IL-23 | | | | | |
| 140 | A10min | R10 PN- | No | Yes | 250 nM | 1 | μM |
| | 6 | IL-23 | | | | | |
| 144 | B10min | R10 PN- | No | Yes | 500 nM | 700 | nM |
| | 4 | IL-23 | | | | | |
| 145 | B10min | R10 PN- | No | Yes | 80 nM | 700 | nM |
| | 5 | IL-23 | | | | | |

| | | | | | | |
|---|---|---|---|---|---|---|
| 151 | Type | N/A | No | Yes | 80 nM | N/A |
| | 1.4 | | | | | |
| 152 | Type | N/A | No | Yes | 80 nM | N/A |
| | 1.5 | | | | | |

### Example 3B

### Cell Based Assay Results for Parent and Minimized Clones from First dRmY Selections

Parent clones from the dRmY selection described in Example 1C, and minimized dRmY clones from this selection (described in Example 2A.2), were tested for activity using the TransAM™ STAT3 activation assay. The three full length dRmY clones described in Example 1C which showed the highest binding affinity for IL-23, ARC489 (SEQ ID NO 91), ARC490 (SEQ ID NO 92), ARC491 (SEQ ID NO 94) were tested. ARC 492 (SEQ ID NO 97) which exhibited no binding to IL-23 was used as a negative control. ARC489 (SEQ ID NO 91), and ARC491 (SEQ ID NO 94) showed comparable cell based activity in the TransAM™ STAT3 activation assay and preliminary data indicate IC₅₀'s in the 50 nM-500 nM range (data not shown).

The only minimized clone from the dRmY minimization efforts described in Example 2A.2 which showed binding to IL-23, ARC527 (SEQ ID NO 159), was tested in the TransAM™ STAT3 activation assay and showed a decrease in assay activity compared to its respective full length ARC489 (SEQ ID NO 91) (data not shown).

### Example 3C

### Cell Based Assay Results for Parent and Minimized Clones from Second dRmY Selections

Parent clones from the dRmY selection described in Example 1D, and minimized clones from this selection (described in Example 2A.3) that displayed high affinity to hIL-23 were screened for functionality in the TransAM™ assay using an 8-point IL-23 titration from 0 to 3 μM in 3 fold dilutions in combination with a constant IL-23 concentration of 3 ng/mL. IC₅₀s for the full length clones were calculated from the dose response curves. FIG. 22 is an example of the dose response curves for the dRmY clones from the selection described in Example 1D that displayed potent cell based activity in the TransAM™ assay (ARC611 (SEQ ID NO 103), ARC614 (SEQ ID NO 105), ARC621 (SEQ ID NO 108), and ARC627 (SEQ ID NO 110)).

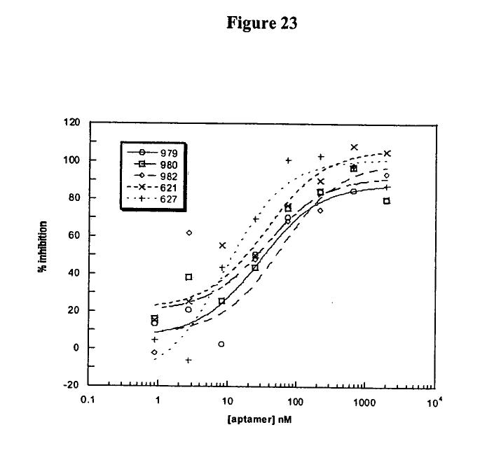

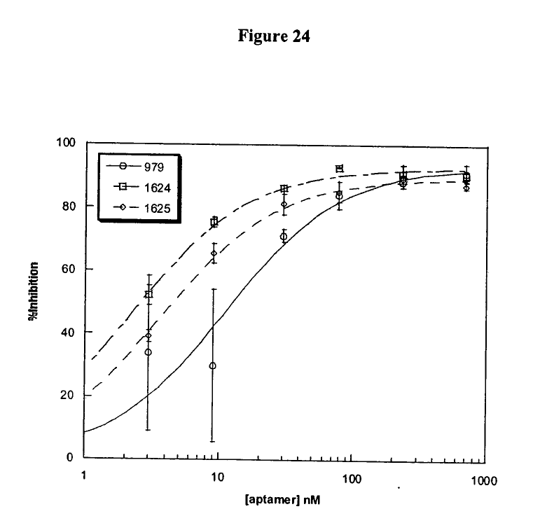

Minimized dRmY clones (described in Example 2A.3) were screened for functionality and compared to their respective parent clone in the in the TransAM™ assay. IC₅₀s were calculated from the dose response curves. FIG. 23 is an example of the dose response curves for some the more potent minimized dRmY clones, ARC979 (SEQ ID NO 177), ARC980 (SEQ ID NO 178), ARC982 (SEQ ID NO 180), compared to the parent full length clones, ARC621 (SEQ ID NO 108) and ARC627 (SEQ ID NO 110). ARC979 (SEQ ID NO 177) consistently performed the best in the TransAM™ assay, with an IC₅₀ of 40 nM+/−10 nM when averaged over the course of three experiments. ARC792 (SEQ ID NO 162), ARC794 (SEQ ID NO 164), ARC795 (SEQ ID NO 165) also displayed potent activity in the TransAM™ assay.

### Example 3D

### Cell Based Assay Results for Optimized ARC979 Derivatives

Several of the optimized ARC979 derivatives described in Example 2B.2 that displayed high affinity to hIL-23 were screened for their ability to inhibit IL-23 induced STAT 3 activation using the PHA Blast assay previously described. Inhibition of IL-23-induced STAT3 phosphorylation was measured using the Pathscan® Phospho-STAT3 (Tyr705) Sandwich ELISA Kit (Cell Signaling Technology, Beverly, Mass.).

Similar to the TransAM™ Assay method previously described, the Pathscan® Phospho-STAT3 (Tyr705) Sandwich ELISA Kit detects endogenous levels of Phospho-STAT3 (Tyr705) protein by using a STAT3 rabbit monoclonal antibody which has been coated onto the wells of a 96-well plate. After incubation with cell lysates, both nonphospho- and phospho-STAT3 proteins are captured by the coated antibody. A phospho-STAT3 mouse monoclonal antibody is added to detect the captured phospho-STAT3 protein, and an HRP-linked anti-mouse antibody is then used to recognize the bound detection antibody. HRP substrate, TMB, is added to develop color, and the magnitude of optical density for this developed color is proportional to the quantity of phospho-STAT3 protein.

PHA Blasts were isolated and prepared as described above and treated with hIL-23 at a final constant concentration of 6 ng/mL (R&D Systems, Minneapolis, Minn.) to induce STAT3 activation, instead of using 3 ng/mL as previously described with the TransAM™ assay. Several clones from the selection described in 2C, were screened by using a 6-point IL-23 titration from 0 to 700 nM in 3 fold dilutions in combination with a constant IL-23 concentration of 6 ng/mL of IL-23 (R&D Systems, Minneapolis, Minn.) to induce STAT3 activation, instead of using 3 ng/mL as previously described with the TransAM™ assay. Lysates of treated cells were prepared using the buffers provided by the Pathscan kit, and the assay was run according to the manufacturer's instructions. IC₅₀s for the full length clones were calculated from the dose response curves.

ARC979, which displayed an IC₅₀ of 40+/−10 nM using the TransAM™ method, consistently displayed an IC₅₀ of 6+/−1 nM using the Pathscan® method. As previously mentioned this IC₅₀ value is consistent with the K_{D} value for ARC979 of 1 nM which was repeatedly verified under the direct binding assay conditions described in Example 2B.2. As can be seen from the Table 36, several of the optimized derivatives of ARC979 remarkably displayed even higher potentcy than ARC979 when directly compared using the Pathscan® Method, particularly ARC1624 and ARC1625, which gave IC₅₀ values of 2 nM and 4 nM respectively.

FIG. 24 is an example of the dose response curves for several of the optimized clones that displayed both high affinity for IL-23 and potent cell based activity in the Pathscan® assay. Table 36 summarizes the IC₅₀'s derived from the dose response curves for the optimized aptamers tested.

TABLE 36
IC₅₀s for Optimized ARC979 derivatives in the Pathscan ® Assay

| | | Pathscan ® IC₅₀ |
|---|---|---|
| SEQ ID NO | Clone | (nM) |
| 177 | 979 | 6 +/− 1 |
| 275 | 1544 | — |
| 308 | 1622 | 9 |
| 309 | 1623 | 5 |
| 310 | 1624 | 2 |
| 311 | 1625 | 4 |
| 312 | 1626 | 12 |
| 313 | 1755 | 68 |
| 314 | 1756 | 19 |

### Example 3E

### Cell Based Assay Results for Parent and Minimized Clones from the Mouse IL-23 Selections

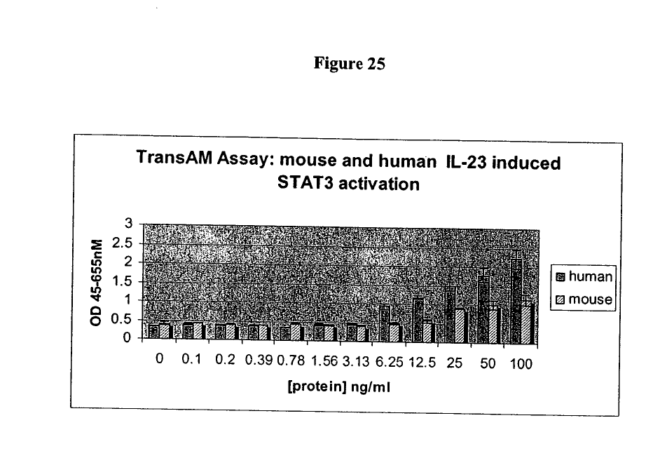

Using the PHA Blast assay and the TransAM™ method described above, mouse IL-23 was shown to activate STAT3 in human PHA blasts (See FIG. 25). Therefore, the ability of the parent clones from the mouse IL-23 selection described in Example 1E, and minimized clones from this selection (described in Example 2A.4) that displayed affinity to mIL-23 to block mouse IL-23 induced STAT3 activation in human PHA blast cells was measured using the TransAM™ assay. The protocol used was identical to that previously described except mouse IL-23 was used to induce STAT 3 activation in PHA Blasts at a concentration of 30 ng/mL, instead of using human IL-23 at a concentration of 3 ng/mL. The results for the parent clones are listed in Table 37 and the results for the minimized clones are listed in Table 38 below.

TABLE 37
Parent mIL-23-rRfY Clone Activity in the TransAM ™ Assay

| SEQ ID NO | Clone Name | Selection | IC₅₀ (nM) |
|---|---|---|---|
| 124 | ARC1628 | R7 mIL-23 | 37 |
| 125 | ARC1629 | R7 mIL-23 | Not Tested |
| 126 | ARC1630 | R7 mIL-23S | 16.6* |
| 127 | ARC1631 | R7 mIL-23S | Not Tested |
| 128 | ARC1632 | R7 mIL-23S | 18 |
| 129 | ARC1633 | R7 mIL-23S | 31 |
| 130 | ARC1634 | R7 mIL-23S | 9 |

*Multiple experiment average.

TABLE 38
Mouse IL-23 rRfY Minimized Clone Activity in the TransAM ™ Assay

| Minimized | | |
|---|---|---|
| Clone | Parent | IC₅₀ mIL-23 |
| SEQ ID NO | Clone | (nM) |
| 199 | ARC1628 | 18 nM |
| 200 | ARC1632 | inactive |
| 201 | ARC1633 | 7 |
| 202 | ARC1634 | 26 |

The invention having now been described by way of written description and example, those of skill in the art will recognize that the invention can be practiced in a variety of embodiments and that the description and examples above are for purposes of illustration and not limitation of the following claims.

What is claimed is:

1) An aptamer that specifically binds to IL-23 comprising the nucleotide sequence of SEQ ID NO: 159.

2) The aptamer of claim 1, further comprising at least one chemical modification.

3) The aptamer of claim 2, wherein the modification is selected from the group consisting: of a chemical substitution at a sugar position; a chemical substitution at a phosphate position; and a chemical substitution at a base position, of the nucleic acid.

4) The aptamer of claim 2, wherein the modification is selected from the group consisting of: incorporation of a modified nucleotide; 3′ capping; conjugation to a high molecular weight, non-immunogenic compound; conjugation to a lipophilic compound; and phosphate backbone modification.

5) The aptamer of claim 4, wherein the high molecular weight, non-immunogenic compound is polyalkylene glycol.

6) The aptamer of claim 5, wherein the polyalkylene glycol is polyethylene glycol.

7) The aptamer of claim 4, wherein the backbone modification comprises incorporation of one or more phosphorothioates into the phosphate backbone.


SEQUENCE LISTING (317 sequences)

SEQ ID NO: 1 - DNA, 93 bp, artificial sequence
  synthetic template
catcgatgctagtcgtaacgatccnnnnnnnnnnnnnnnnnnnnnnnnnnnnnncgagaacgttctctcctctccctatagtgagtcgtatta

SEQ ID NO: 2 - DNA, 92 bp, artificial sequence
  synthetic template
catgcatcgcgactgactagccgnnnnnnnnnnnnnnnnnnnnnnnnnnnnnngtagaacgttctctcctctccctatagtgagtcgtatta

SEQ ID NO: 3 - DNA, 92 bp, artificial sequence
  synthetic template
catcgatcgatcgatcgacagcgnnnnnnnnnnnnnnnnnnnnnnnnnnnnnngtagaacgttctctcctctccctatagtgagtcgtatta

SEQ ID NO: 4 - protein, 328 aa, homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 189 aa, homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 6 - protein, 253 aa, homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - DNA, 10 bp, artificial sequence
  synthetic CpG
aacgttcgag

SEQ ID NO: 8 - RNA, 88 nt, artificial sequence
  synthetic template
gggaaaagcgaaucauacacaagannnnnnnnnnnnnnnnnnnnnnnnnnnnnnnnnnnnnnnngcuccgccagagaccaaccgagaa

SEQ ID NO: 9 - DNA, 41 bp, artificial sequence
  synthetic primer
taatacgactcactatagggaaaagcgaatcatacacaaga

SEQ ID NO: 10 - DNA, 24 bp, artificial sequence
  synthetic primer
ttctcggttggtctctggcggagc

SEQ ID NO: 11 - RNA, 24 nt, artificial sequence
  synthetic nucleic acid sequence
gggaaaagcgaaucauacacaaga

SEQ ID NO: 12 - RNA, 24 nt, artificial sequence
  synthetic nucleic acid sequence
gcuccgccagagaccaaccgagaa

SEQ ID NO: 13 - RNA, 88 nt, artificial sequence
  synthetic aptamer
gggaaaagcgaaucauacacaagagagguaugugguuuugcggagcaacucgugucagcggucagcuccgccagagaccaaccgagaa

SEQ ID NO: 14 - RNA, 88 nt, artificial sequence
  synthetic aptamer
gggaaaagcgaaucauacacaagaaugaauuccguccacgggcgcccgaugaugucaguuuucggcuccgccagagaccaaccgagaa

SEQ ID NO: 15 - RNA, 88 nt, artificial sequence
  synthetic aptamer
gggaaaagcgaaucauacacaagauuagugcguguguugaaagggcucauaaugucaguaucgagcuccgccagagaccaaccgagaa

SEQ ID NO: 16 - RNA, 88 nt, artificial sequence
  synthetic aptamer
gggaaaagcgaaucauacacaagauuaggcgucgugacaauaacugguccacgagcaugucagugcuccgccagagaccaaccgagaa

SEQ ID NO: 17 - RNA, 88 nt, artificial sequence
  synthetic aptamer
gggaaaagcgaaucauacacaagauggaaggcgaucguagcaguaacccaaugauugggaccuagcuccgccagagaccaaccgagaa

SEQ ID NO: 18 - RNA, 88 nt, artificial sequence
  synthetic aptamer
gggaaaagcgaaucauacacaagaucucuuuggccgacgcaacaaugcucuuuuccgaccuugcgcuccgccagagaccaaccgagaa

SEQ ID NO: 19 - RNA, 89 nt, Artificial Sequence
  synthetic aptamer
gggaaaagcgaauccuacccaagauguuguuggcguugaucguaugauunauggagngugucngugcuccgccagagaccaaccgagaa

SEQ ID NO: 20 - RNA, 88 nt, artificial sequence
  synthetic aptamer
gggaaaagcgaaucauacacaagaugcgcuauguuuggcugggaauuguagcauugcucaaguggcuccgccagagaccaaccgagaa

SEQ ID NO: 21 - RNA, 88 nt, artificial sequence
  synthetic aptamer
gggaaaagcgaaucauacacaagauguugaaccucuugugcgucccgauguuungcaauguggagcuccgccagagaccaaccgagaa

SEQ ID NO: 22 - RNA, 88 nt, artificial sequence
  synthetic aptamer
gggaaaagcgaaucauacacaagaauguauacaaugcccuaucgucaguuaggcauguguggaugcuccgccagagaccaaccgagaa

SEQ ID NO: 23 - RNA, 89 nt, artificial sequence
  synthetic aptamer
gggaaaagcgaaucauacacaagacagaggcaaugagagccuggcgaugucagucgcaucuugcugcuccgccagagaccaaccgagaa

SEQ ID NO: 24 - RNA, 88 nt, artificial sequence
  synthetic aptamer
gggaaaagcgaaucauacacaagaucgcaaaaggaguuugucucugcucucggagugugucagugcuccgccagagaccaaccgagaa

SEQ ID NO: 25 - RNA, 88 nt, artificial sequence
  synthetic aptamer
gggaaaagcgaaucauacacaagagaugacuacacgccagugugcgcuuuuugcggaguuagcggcuccgccagagaccaaccgagaa

SEQ ID NO: 26 - RNA, 89 nt, artificial sequence
  synthetic aptamer
gggaaaagcgaaucauacacaagagucgugaugauuuggguuaugucaguucccuguaugguuucgcuccgccagagaccaaccgagaa

SEQ ID NO: 27 - RNA, 88 nt, artificial sequence
  synthetic aptamer
gggaaaagcgaaucauacacaagaguuuuaugugggucccgaugauuaacuuuauuggcgcauugcuccgccagagaccaaccgagaa

SEQ ID NO: 28 - RNA, 90 nt, artificial sequence
  synthetic aptamer
gggaaaagcgaaucauacacaagagaacgaguauauuugcgcuggcggagaagucucucgaagggagcuccgccagagaccaaccgagaa

SEQ ID NO: 29 - RNA, 88 nt, artificial sequence
  synthetic aptamer
gggaaaagcgaaucauacacaagaguaucauucggcuggugggagaaaucucuguagauauagagcuccgccagagaccaaccgagaa

SEQ ID NO: 30 - RNA, 88 nt, artificial sequence
  synthetic aptamer
gggaaaagcgaaucauacacaagauagcgucuaugauggcggagaagcaaguguagcauaacaggcuccgccagagaccaaccgagaa

SEQ ID NO: 31 - RNA, 88 nt, artificial sequence
  synthetic aptamer
gggaaaagcgaaucauacacaagaguguugaaugagcgcugguggacagaucuuugguuacagagcuccgccagagaccaaccgagaa

SEQ ID NO: 32 - RNA, 88 nt, artificial sequence
  synthetic aptamer
gggaaaagcgaaucauacacaagacucauggauauggccuagcagccguggaagcggucauucugcuccgccagagaccaaccgagaa

SEQ ID NO: 33 - RNA, 88 nt, artificial sequence
  synthetic aptamer
gggaaaagcgaaucauacacaagaucccagcgguacgugagucuguuaaaggccaccuaaugucgcuccgccagagaccaaccgagaa

SEQ ID NO: 34 - RNA, 87 nt, artificial sequence
  synthetic aptamer
gggaaaagcgaaucauacacaagaguaaugugggucccgaugauucgcugugcggcguuuguagcuccgccagagaccaaccgagaa

SEQ ID NO: 35 - RNA, 88 nt, artificial sequence
  synthetic aptamer
gggaaaagcgaaucauacacaagagguugaguacgacggagucnuggcuaacacggaaacuagagcuccgccagagaccaaccgagaa

SEQ ID NO: 36 - RNA, 88 nt, artificial sequence
  synthetic aptamer
gggaaaagcgaaucauacacaagagucauggcuuacaauugaaacaagagcucgcgugacacaugcuccgccagagaccaaccgagaa

SEQ ID NO: 37 - RNA, 88 nt, artificial sequence
  synthetic aptamer
gggaaaagcgaaucauacacaagaacggcuaggcaucaauggccagcaaaaauagucguguaaugcuccgccagagaccaaccgagaa

SEQ ID NO: 38 - RNA, 88 nt, artificial sequence
  synthetic aptamer
gggaaaagcgaaucauacacaagaccaucggacgaggcgggucaccuuuuacgcuuucgagcuggcuccgccagagaccaaccgagaa

SEQ ID NO: 39 - RNA, 88 nt, artificial sequence
  synthetic aptamer
gggaaaagcgaaucauacacaagaugguucccacgugaaaguggcuagcgaguaccccacuuaugcuccgccagagaccaaccaaggg

SEQ ID NO: 40 - RNA, 90 nt, artificial sequence
  synthetic aptamer
gggaaaagcgaaucauacacaagagcgcuuuagcggguauagcacuuuucaucuaaugaanccguagcuccgccagagaccaaccgagaa

SEQ ID NO: 41 - RNA, 88 nt, artificial sequence
  synthetic aptamer
gggaaaagcgaaucauacacaagaucuacgauuguucagguuuuuuguacucaacuaaaggcgagcuccgccagagaccaaccgagaa

SEQ ID NO: 42 - RNA, 88 nt, artificial sequence
  synthetic aptamer
gggaaaagcgaaucauacacaagauugucucggauuggucacucccauuuuuguucgcuuaacggcuccgccagagaccaaccgagaa

SEQ ID NO: 43 - RNA, 89 nt, artificial sequence
  synthetic aptamer
gggaaaagcgaaucauacacaagaaguuuuuugugcucugaguacucagcguccguaagggauaugcuccgccagagaccaaccgagaa

SEQ ID NO: 44 - RNA, 88 nt, artificial sequence
  synthetic aptamer
gggaaaagcgaaucauacacaagaagugcuucaugcggcaaacugcaugacgucgaauagauaugcuccgccagagaccaaccgagaa

SEQ ID NO: 45 - RNA, 88 nt, artificial sequence; identical to SEQ ID NO: 13
  synthetic aptamer

SEQ ID NO: 46 - RNA, 88 nt, artificial sequence
  synthetic aptamer
gggaaaagcgaaucauacacaagaugugcuugaguuaaaucucaucguccccguuuggggauaugcuccgccagagaccaaccgagaa

SEQ ID NO: 47 - RNA, 88 nt, artificial sequence
  synthetic aptamer
gggaaaagcgaaucauacacaagaaguuuuugugcucugaguacucagcguccguaagggauaugcuccgccagagaccaaccgagaa

SEQ ID NO: 48 - RNA, 88 nt, artificial sequence
  synthetic aptamer
gggaaaagcgaaucauacacaagagauguauucaggcgguccgcauugaugucaguuaugcguagcuccgccagagaccaaccgagaa

SEQ ID NO: 49 - RNA, 88 nt, artificial sequence
  synthetic aptamer
gggaaaagcgaaucauacacaagaauggucggaaucucuggcgccacgcugaguauagacggaagcuccgccagagaccaaccgagaa

SEQ ID NO: 50 - RNA, 88 nt, artificial sequence
  synthetic aptamer
gggaaaagcgaaucauacacaagagugcuucguauguugaauacgacguucgcaggacgaauaugcuccgccagagaccaaccgagaa

SEQ ID NO: 51 - RNA, 87 nt, artificial sequence
  synthetic aptamer
agggaaaaggaaucauacacaagauguaucauccggucguacaaaagcgccacggaaccauucgcuccgccaganaccaaccgagaa

SEQ ID NO: 52 - RNA, 88 nt, artificial sequence
  synthetic aptamer
gggaaaagcgaaucauacacaagacgcgucagguccacgcugaaauuuauuuucggcaguguaagcuccgccagagaccaaccgagaa

SEQ ID NO: 53 - RNA, 88 nt, artificial sequence
  synthetic aptamer
gggaaaagcgaaucauacacaagauaugugccugggauggacgacauccccugucuaaggauaugcuccgccagagaccaaccgagaa

SEQ ID NO: 54 - RNA, 88 nt, artificial sequence
  synthetic aptamer
gggaaaagcgaaucauacacaagauuacuccguuagugucaguugacggagggagcguacuauugcuccgccagagaccaaccgagaa

SEQ ID NO: 55 - RNA, 88 nt, artificial sequence
  synthetic aptamer
gggaaaagcgaaucauacacaagacauugugcuuuaucacgugggugauaacgacgaaaguuaugcuccgccagagaccaaccgagaa

SEQ ID NO: 56 - RNA, 89 nt, artificial sequence
  synthetic aptamer
gggaaaagcgaaucauacacaagacaguguaugaggaagauuacuuccauuccugagcgguuuucgcuccgccagagaccaaccgagaa

SEQ ID NO: 57 - RNA, 88 nt, artificial sequence
  synthetic aptamer
gggaaaagcgaaucauacacaagauuggcaaugugaccuucaacccuuuucccgaugaacaguggcuccgccagagaccaaccgagaa

SEQ ID NO: 58 - RNA, 88 nt, artificial sequence
  synthetic aptamer
gggaaaagcgaaucauacacaagacaugacugcaugcuucgggaguaucucggucccgacguucgcuccgccagagaccaaccgagaa

SEQ ID NO: 59 - RNA, 88 nt, artificial sequence
  synthetic aptamer
gggaaaagcgaaucauacacaagacuuaucgccucaagggggguaauaaacccagcgugugcaugcuccgccagagaccaaccgagaa

SEQ ID NO: 60 - RNA, 89 nt, artificial sequence
  synthetic aptamer
gggaaaagcgaaucauacacaagaauccuggcuucgcauaguguauggguaguacgacagcgcgugcuccgccagagaccaaccgagaa

SEQ ID NO: 61 - RNA, 89 nt, artificial sequence
  synthetic aptamer
gggaaaagcgaaucauacacaagaacgcauagucggauuuaccgaucauucugugccuucgugacgcuccgccagagaccaaccgagaa

SEQ ID NO: 62 - RNA, 88 nt, artificial sequence
  synthetic aptamer
gggaaaagcgaaucauacacaagaauugugcuuacaacuuucguuguaccgacgugucaguuaugcuccgccagagaccaaccgagaa

SEQ ID NO: 63 - RNA, 88 nt, artificial sequence
  synthetic aptamer
gggaaaagcgaaucauacacaagaguguauuacccccaacccagggggaccauucgcguaacaagcuccgccagagaccaaccgagaa

SEQ ID NO: 64 - RNA, 88 nt, artificial sequence
  synthetic aptamer
gggaaaagcgaaucauacacaagacuuaacagugcggggcgcaguguauagauccgcaaugugugcuccgccagagaccaaccgagaa

SEQ ID NO: 65 - RNA, 88 nt, artificial sequence
  synthetic aptamer
gggaaaagcgaaucauacacaagacgauaguaugaccuuuugaaaggcuucccgagcgguguucgcuccgccagagaccaaccgagaa

SEQ ID NO: 66 - RNA, 88 nt, artificial sequence
  synthetic aptamer
gggaaaagcgaaucauacacaagacgugugcuuuauguaaaccauaacguuccauaaggaauaugcuccgccagagaccaaccgagaa

SEQ ID NO: 67 - DNA, 39 bp, artificial sequence
  synthetic primer
taatacgactcactatagggagaggagagaacgttctac

SEQ ID NO: 68 - DNA, 22 bp, artificial sequence
  synthetic primer
catcgatcgatcgatcgacagc

SEQ ID NO: 69 - RNA, 22 nt, artificial sequence
  synthetic nucleic acid sequence
gggagaggagagaacguucuac

SEQ ID NO: 70 - RNA, 22 nt, artificial sequence
  synthetic nucleic acid sequence
gcugucgaucgaucgaucgaug

SEQ ID NO: 71 - RNA, 74 nt, artificial sequence
  synthetic aptamer
gggagaggagagaacguucuacaaaugagagcaggccgaagaggagucgcucgcugucgaucgaucgaucgaug

SEQ ID NO: 72 - RNA, 74 nt, artificial sequence
  synthetic aptamer
gggagaggagagaacguucuacaaaugagagcaggccgaaaaggagucgcucgcugucgaucgaucgaucgaug

SEQ ID NO: 73 - RNA, 74 nt, artificial sequence; identical to SEQ ID NO: 72
  synthetic aptamer

SEQ ID NO: 74 - RNA, 75 nt, artificial sequence
  synthetic aptamer
gggagaggagagaacguucuacgguaaagcaggcugacugaaagguugaagucgcugucgaucgaucgaucgaug

SEQ ID NO: 75 - RNA, 74 nt, artificial sequence
  synthetic aptamer
gggagaggagagaacguucuacagguuaagagcaggcucaggaauggaagucgcugucgaucgaucgaucgaug

SEQ ID NO: 76 - RNA, 75 nt, artificial sequence
  synthetic aptamer
gggagaggagagaacguucuacaacaaagcaggcucauaguaauauggaagucgcugucgaucgaucgaucgaug

SEQ ID NO: 77 - RNA, 75 nt, artificial sequence; identical to SEQ ID NO: 76
  synthetic aptamer

SEQ ID NO: 78 - RNA, 74 nt, artificial sequence
  synthetic aptamer
gggagaggagagaacguucuacaaaagagagcaggccgaaaaggagucgcucgcugucgaucgaucgaucgaug

SEQ ID NO: 79 - RNA, 75 nt, artificial sequence
  synthetic aptamer
gggagaggagagaacguucuacaaaaggcaggcucaggggaucacuggaagucgcugucgaucgaucgaucgaug

SEQ ID NO: 80 - RNA, 76 nt, artificial sequence
  synthetic aptamer
gggagaggagagaacguucuacaagauauaauuaaggauaagugcaaaggagacgcugucgaucgaucgaucgaug

SEQ ID NO: 81 - RNA, 74 nt, artificial sequence
  synthetic aptamer
gggagaggagagaacguucuacgaaugagagcaggccgaaaaggagucgcucgcugucgaucgaucgaucgaug

SEQ ID NO: 82 - RNA, 75 nt, artificial sequence
  synthetic aptamer
gggagaggagagaacguucuacgagaggcaagagagagucgcauaaaaaagacgcugucgaucgaucgaucgaug

SEQ ID NO: 83 - RNA, 75 nt, artificial sequence
  synthetic aptamer
gggagaggagagaacguucuacgcaggcugucguagacaaacgaugaagucgcgcugucgaucgaucgaucgaug

SEQ ID NO: 84 - RNA, 76 nt, artificial sequence
  synthetic aptamer
gggagaggagagaacguucuacggaaaaagauaugaaagaaaggauuaagagacgcugucgaucgaucgaucgaug

SEQ ID NO: 85 - RNA, 75 nt, artificial sequence
  synthetic aptamer
gggagaggagagaacguucuacggaaggnaacaanagcacuguuugugcaggcgcugucgaucnaucnaucnaug

SEQ ID NO: 86 - RNA, 73 nt, artificial sequence
  synthetic aptamer
gggagaggagagaacguucuacuaaugcaggcucaguuacuacuggaagucgcugucgaucgaucgaucgaug

SEQ ID NO: 87 - RNA, 75 nt, artificial sequence
  synthetic aptamer
aggagaggagagaacguucuacuagaagcaggcucgaauacaauucggaagucgcugucgaucgaucgaucgaug

SEQ ID NO: 88 - RNA, 75 nt, artificial sequence
  synthetic aptamer
gggagaggagagaacguucuacauaagcaggcuccgauaguauucgggaagucgcugucgaucgaucgaucgaug

SEQ ID NO: 89 - DNA, 22 bp, artificial sequence
  synthetic  primer
catcgatcgatcgatcgacagc

SEQ ID NO: 90 - RNA, 22 nt, artificial sequence
  synthetic nucleic acid sequence
gcugucgaucgaucgaucgaug

SEQ ID NO: 91 - DNA, 74 bp, artificial sequence
  synthetic aptamer
gggagaggagagaacguucuacagcgccggugggcgggcauuggguggaugcgcugucgaucgaucgaucgaug

SEQ ID NO: 92 - DNA, 75 bp, artificial sequence
  synthetic aptamer
gggagaggagagaacguucuacagccuuuuggguaaggggaggggugccggucgcugucgaucgaucgaucgaug

SEQ ID NO: 93 - DNA, 75 bp, artificials sequence
  modified_base (1)..(75): all purines (A and G) are deoxy, all pyrimidines (C and U) are 2′-O-methyl
gggagaggagagaacguucuacguaacggggugggaggggcgaacaacuugacgcugucgaucgaucgaucgaug

SEQ ID NO: 94 - DNA, 74 bp, artificial sequence
  synthetic aptamer
gggagaggagagaacguucuacagcgccggugggugggcauaggguggaugcgcugucgaucgaucgaucgaug

SEQ ID NO: 95 - DNA, 75 bp, artificial sequence
  synthetic aptamer
gggagaggagagaacguucuacgggcuacggggauggagggugggucccagacgcugucgaucgaucgaucgaug

SEQ ID NO: 96 - DNA, 75 bp, artificial sequence
  synthetic aptamer
gggagaggagagaacguucuacacggggugggaggggcgagucgcauggaugcgcugucgaucgaucgaucgaug

SEQ ID NO: 97 - DNA, 75 bp, artificial sequence
  synthetic aptamer
gggagaggagagaacguucuacucaaugaccgcgcgaggcucugggagagggcgcugucgaucgaucgaucgaug

SEQ ID NO: 98 - DNA, 75 bp, artificial sequence
  synthetic template
gggagaggagagaacguucuacnnnnnnnnnnnnnnnnnnnnnnnnnnnnnnggucgaucgaucgaucaucgaug

SEQ ID NO: 99 - DNA, 39 bp, artificial sequence; identical to SEQ ID NO: 67
  synthetic primer

SEQ ID NO: 100 - DNA, 22 bp, artificial sequence
  synthetic primer
catcgatgatcgatcgatcgac

SEQ ID NO: 101 - RNA, 22 nt, artificial sequence
  synthetic nucleic acid sequence
gggagaggagagaacguucuac

SEQ ID NO: 102 - RNA, 22 nt, artificial sequence
  synthetic nucleic acid sequence
gucgaucgaucgaucaucgaug

SEQ ID NO: 103 - DNA, 75 bp, artificial sequence
  synthetic aptamer
gggagaggagagaacguucuacaggcaaggcaauuggggaguguggguggggggucgaucgaucgaucaucgaug

SEQ ID NO: 104 - DNA, 75 bp, artificial sequence
  synthetic aptamer
gggagaggagagaacguucuacaggcaaguaauuggggagugcgggcgggggggucgaucgaucgaucaucgaug

SEQ ID NO: 105 - DNA, 75 bp, artificial sequence
  synthetic aptamer
gggagaggagagaacguucuacaaggcgguacggggaguguggguuggggccggucgaucgaucgaucaucgaug

SEQ ID NO: 106 - DNA, 75 bp, artificial sequence
  synthetic aptamer
gggagaggagagaacguucuacgauauaggcgguacggggggagugggcuggggucgaucgaucgaucaucgaug

SEQ ID NO: 107 - DNA, 75 bp, artificial sequence
  synthetic aptamer
gggagaggagagaacguucuacaggaaaggcgcuugcggggggugagggaggggucgaucgaucgaucaucgaug

SEQ ID NO: 108 - DNA, 74 bp, artificial sequence
  synthetic aptamer
gggagaggagagaacguucuacaggcgguuacgggggaugcgggugggacaggucgaucgaucgaucaucgaug

SEQ ID NO: 109 - DNA, 74 bp, artificial sequence
  synthetic aptamer
gggagaggagagaacguucuacaggcaaguaauuggggagugcgggcggggggucgaucgaucgaucaucgaug

SEQ ID NO: 110 - DNA, 74 bp, artificial sequence
  synthetic aptamer
gggagaggagagaacguucuacaggcaaguaauuggggagugcgggcggggugucgaucgaucgaucaucgaug

SEQ ID NO: 111 - DNA, 76 bp, artificial sequence
  synthetic aptamer
gggagaggagagaacguucuacaggcaaggcaauuggggagcgugggugggggggucgaucgaucgaucaucgaug

SEQ ID NO: 112 - DNA, 75 bp, artificial sequence
  synthetic aptamer
gggagaggagagaacguucuacaauugcagguggugccggggguugggggcgggucgaucgaucgaucaucgaug

SEQ ID NO: 113 - DNA, 73 bp, artificial sequence
  synthetic aptamer
gggagaggagagaacguucuacaggcucaaaagagggggaugugggagggggucgaucgaucgaucaucgaug

SEQ ID NO: 114 - DNA, 74 bp, artificial sequence
  synthetic aptamer
gggagaggagagaacguucuacaggcgcagccagcggggagugagggugggggucgaucgaucgaucaucgaug

SEQ ID NO: 115 - DNA, 74 bp, artificial sequence
  synthetic aptamer
gggagaggagagaacguucuacaggccgaugagggggagcaguggguggggggucgaucgaucgaucaucgaug

SEQ ID NO: 116 - DNA, 75 bp, artificial sequence
  synthetic aptamer
gggagaggagagaacguucuacuagugaggcgguaacggggggugaggguggggucgaucgaucgaucaucgaug

SEQ ID NO: 117 - DNA, 76 bp, artificial sequence
  synthetic aptamer
gggagaggagagaacguucuacagguaggcaagauauugggggaagcggguggggucgaucgaucgaucaucgaug

SEQ ID NO: 118 - DNA, 75 bp, artificial sequence
  synthetic aptamer
gggagaggagagaacguucuacacauggcucgaaagaggggcgugaggguggggucgaucgaucgaucaucgaug

SEQ ID NO: 119 - RNA, 76 nt, artificial sequence
  synthetic template
ggagcgcacucagccacnnnnnnnnnnnnnnnnnnnnnnnnnnnnnnnnnnnnnnnnuuucgaccucucugcuagc

SEQ ID NO: 120 - DNA, 34 bp, artificial sequence
  synthetic primer
taatacgactcactataggagcgcactcagccac

SEQ ID NO: 121 - DNA, 19 bp, artificial sequence
  synthetic primer
gctagcagagaggtcgaaa

SEQ ID NO: 122 - RNA, 17 nt, artificial sequence
  synthetic nucleic acid sequence
ggagcgcacucagccac

SEQ ID NO: 123 - RNA, 19 nt, artificial sequence
  synthetic nucleic acid sequence
uuucgaccucucugcuagc

SEQ ID NO: 124 - RNA, 75 nt, artificial sequence
  synthetic aptamer
ggagcgcacucagccacagguggcuuaauacuguaaagacgugcgcgcagagggauuuucgaccucucugcuagc

SEQ ID NO: 125 - RNA, 76 nt, artificial sequence
  synthetic aptamer
ggagcgcacucagccaccguaauucacaaggucccugagugcaggguuguauguuuguuucgaccucucugcuagc

SEQ ID NO: 126 - RNA, 76 nt, artificial sequence
  synthetic aptamer
ggagcgcacucagccacucuacucgauauaguuuaucgagccggugguagauuaugauuucgaccucucugcuagc

SEQ ID NO: 127 - RNA, 75 nt, artificial sequence
  synthetic aptamer
ggagcgcacucagccacgccuacaauucacugugauauaucgaauuauagcccugguuucgaccucucugcuagc

SEQ ID NO: 128 - RNA, 76 nt, artificial sequence
  synthetic aptamer
ggagcgcacucagccaccggcuuaauauccaauaggaacguucgcucugagcaggcguuucgaccucucugcuagc

SEQ ID NO: 129 - RNA, 76 nt, artificial sequence
  synthetic aptamer
ggagcgcacucagccacagcucgguggcuuaauaucuaugugaacgugcgcaacagcuuucgaccucucugcuagc

SEQ ID NO: 130 - RNA, 76 nt, artificial  sequence
  synthetic aptamer
ggagcgcacucagccaccuugggcuuaauaccuaucggaugugcgccuagcacggaauuucgaccucucugcuagc

SEQ ID NO: 131 - RNA, 76 nt, artificial sequence
  synthetic aptamer
ggagcgcacucagccacgguuuacuuccguggcaauauugaccucncucuagacagguuucgaccucucugcuagc

SEQ ID NO: 132 - RNA, 76 nt, artificial sequence
  synthetic aptamer
ggagcgcacucagccaccugggaaaaucugggucccugaguucuaacagcagagauuuuucgaccucucugcuagc

SEQ ID NO: 133 - RNA, 76 nt, artificial sequence
  synthetic aptamer
ggagcgcacucngccacuucggaauaucguugucuucugggugagcaugcguugagguuucnaccucucugcuagc

SEQ ID NO: 134 - RNA, 76 nt, artificial sequence
  synthetic aptamer
ggagcgcacucagccacuggggaacaucucaugucucugaccgcucuugcaguagaauuungaccucucugcuagc

SEQ ID NO: 135 - RNA, 60 nt, artificial sequence
  synthetic aptamer
ggagaucauacacaagaaguuuuuugugcucugaguacucagcguccguaagggaucucc

SEQ ID NO: 136 - RNA, 48 nt, artificial sequence
  synthetic aptamer
ggagucugaguacucagcguccguaagggauaugcuccgccagacucc

SEQ ID NO: 137 - RNA, 39 nt, artificial sequence
  synthetic aptamer
ggaguuacucagcguccguaagggauaugcuccgacucc

SEQ ID NO: 138 - RNA, 50 nt, artificial sequence
  synthetic aptamer
ggagucugaguacucagcgucccgagaggggauaugcuccgccagacucc

SEQ ID NO: 139 - RNA, 61 nt, artificial sequence
  synthetic aptamer
ggagcauacacaagaaguuuuuugugcucugaguacucagcguccguaagggauaugcucc

SEQ ID NO: 140 - RNA, 51 nt, artificial sequence
  synthetic aptamer
ggaguacgccgaaaggcgcucugaguacucagcguccguaagggauacucc

SEQ ID NO: 141 - RNA, 65 nt, artificial sequence
  synthetic aptamer
ggagcgaaucauacacaagaagugcuucaugcggcaaacugcaugacgucgaauagauaugcucc

SEQ ID NO: 142 - RNA, 55 nt, artificial sequence
  synthetic aptamer
ggaucauacacaagaagugcuucaugcggcaaacugcaugacgucgaauagaucc

SEQ ID NO: 143 - RNA, 47 nt, artificial sequence
  synthetic aptamer
ggaucauacacaagaagugcuucacgaaagugacgucgaauagaucc

SEQ ID NO: 144 - RNA, 60 nt, artificial sequence
  synthetic aptamer
ggagcauacacaagaagugcuucaugcggcaaacugcaugacgucgaauagauaugcucc

SEQ ID NO: 145 - RNA, 46 nt, artificial sequence
  synthetic aptamer
ggaguacacaagaagugcuuccgaaaggacgucgaauagauacucc

SEQ ID NO: 146 - RNA, 30 nt, artificial sequence
  synthetic aptamer
gguuaaaucucaucguccccguuuggggau

SEQ ID NO: 147 - RNA, 57 nt, artificial sequence
  synthetic aptamer
ggacauacacaagaugugcuugaguuaaaucucaucguccccguuuggggauauguc

SEQ ID NO: 148 - RNA, 47 nt, artificial sequence
  synthetic aptamer
ggcauacacgagagugcugucgaaagacucggccgagaggcuaugcc

SEQ ID NO: 149 - RNA, 47 nt, artificial sequence
  synthetic aptamer
ggcauacgcgagagcgcuggcgaaagccucggccgagaggcuaugcc

SEQ ID NO: 150 - RNA, 43 nt, artificial sequence
  synthetic aptamer
ggauacccgagagggcuggcgaaagccucggcgagagcuaucc

SEQ ID NO: 151 - RNA, 47 nt, artificial sequence
  synthetic aptamer
ggguacgccgaaaggcgcuuccgaaaggacguccguaagggauaccc

SEQ ID NO: 152 - RNA, 49 nt, artificial sequence
  synthetic aptamer
ggaguacgccgaaaggcgcuuccgaaaggacguccguaagggauacucc

SEQ ID NO: 153 - RNA, 42 nt, artificial sequence
  synthetic aptamer
ggaaucauaccgagagguauuaccccgaaaggggaccauucc

SEQ ID NO: 154 - RNA, 48 nt, artificial sequence
  synthetic aptamer
ggaaucauacacaagaguguauuacccccaacccagggggaccauucc

SEQ ID NO: 155 - RNA, 57 nt, artificial sequence
  synthetic aptamer
ggaagaauggucggaaucucuggcgccacgcugaguauagacggaagcuccgccaga

SEQ ID NO: 156 - RNA, 39 nt, artificial sequence
  synthetic aptamer
ggaggcgccacgcugaguauagacggaagcuccgccucc

SEQ ID NO: 157 - RNA, 58 nt, artificial sequence
  synthetic aptamer
ggacacaagagauguauucaggcgguccgcauugaugucaguuaugcguagcuccgcc

SEQ ID NO: 158 - RNA, 38 nt, artificial sequence
  synthetic aptamer
ggcgguccgcauugaugucaguuaugcguagcuccgcc

SEQ ID NO: 159 - DNA, 37 bp, artificial sequence
  synthetic aptamer
acagcgccggugggcgggcauuggguggaugcgcugu

SEQ ID NO: 160 - DNA, 32 bp, artificial sequence
  synthetic aptamer
gcgccggugggcgggcaccggguggaugcgcc

SEQ ID NO: 161 - DNA, 35 bp, artificial sequence
  synthetic aptamer
acagcgccgguguuuucauuggguggaugcgcugu

SEQ ID NO: 162 - DNA, 28 bp, artificial sequence
  synthetic aptamer
ggcaaguaauuggggagugcgggcgggg

SEQ ID NO: 163 - DNA, 25 bp, artificial sequence
  synthetic aptamer
cuacaaggcgguacggggagugugg

SEQ ID NO: 164 - DNA, 30 bp, artificial sequence
  synthetic aptamer
ggcgguacggggaguguggguuggggccgg

SEQ ID NO: 165 - DNA, 36 bp, artificial sequence
  synthetic aptamer
cgauauaggcgguacggggggagugggcuggggucg

SEQ ID NO: 166 - DNA, 31 bp, artificial sequence
  synthetic aptamer
uaauuggggagugcgggcggggggucgaucg

SEQ ID NO: 167 - DNA, 27 bp, artificial sequence
  synthetic aptamer
gguggggagugcgggcggggggucgcc

SEQ ID NO: 168 - DNA, 35 bp, artificial sequence
  synthetic aptamer
acaggcaagguaauuggggagugcgggcggggugu

SEQ ID NO: 169 - DNA, 35 bp, artificial sequence
  synthetic aptamer
ccaggcaagguaauuggggagugcgggcggggugg

SEQ ID NO: 170 - DNA, 29 bp, artificial sequence
  synthetic aptamer
ggcaagguaauugggaagugugggcgggg

SEQ ID NO: 171 - DNA, 29 bp, artificial sequence
  synthetic aptamer
ggcaagguaauuggguagugagggcgggg

SEQ ID NO: 172 - DNA, 29 bp, artificial sequence
  synthetic aptamer
ggcaagguaauuggggagugcgggcuggg

SEQ ID NO: 173 - DNA, 29 bp, artificial sequence
  synthetic aptamer
ggcaagguaauugggaagugugggcuggg

SEQ ID NO: 174 - DNA, 29 bp, artificial sequence
  synthetic aptamer
ggcaagguaauuggguagugagggcuggg

SEQ ID NO: 175 - DNA, 35 bp, artificial sequence
  synthetic aptamer
acaggcaagguaauuggguagugagggcugggugu

SEQ ID NO: 176 - DNA, 41 bp, artificial sequence
  synthetic aptamer
gauguuggcaaguaauuggggagugcgggcgggguucauct

SEQ ID NO: 177 - DNA, 34 bp, artificial sequence
  synthetic aptamer
acaggcaaguaauuggggagugcgggcggggugu

SEQ ID NO: 178 - DNA, 34 bp, artificial sequence
  synthetic aptamer
ccaggcaaguaauuggggagugcgggcggggugg

SEQ ID NO: 179 - DNA, 25 bp, artificial sequence
  synthetic aptamer
ggcgguuacgggggaugcggguggg

SEQ ID NO: 180 - DNA, 30 bp, artificial sequence
  synthetic aptamer
ggcgguuacgggggaugcgggugggacagg

SEQ ID NO: 181 - DNA, 26 bp, artificial sequence
  synthetic aptamer
ggcaaguaauuggggagugcgggcgg

SEQ ID NO: 182 - DNA, 32 bp, artificial sequence
  synthetic aptamer
acaggcaaguaauuggggagugcgggcggugu

SEQ ID NO: 183 - DNA, 28 bp, artificial sequence
  synthetic aptamer
ggcgguacggggaguguggguuggggcc

SEQ ID NO: 184 - DNA, 28 bp, artificial sequence
  synthetic aptamer
ggcgguacggggagugugggcuggggcc

SEQ ID NO: 185 - DNA, 22 bp, artificial sequence
  synthetic aptamer
gguacggggaguguggguuggg

SEQ ID NO: 186 - DNA, 22 bp, artificial sequence
  synthetic aptamer
gguacggggagugugggcuggg

SEQ ID NO: 187 - DNA, 27 bp, artificial sequence
  synthetic aptamer
ggcgguacggggaguguggguugggcc

SEQ ID NO: 188 - DNA, 27 bp, artificial sequence
  synthetic aptamer
ggcgguacggggagugugggcugggcc

SEQ ID NO: 189 - DNA, 21 bp, artificial sequence
  synthetic aptamer
gguacggggaguguggguugg

SEQ ID NO: 190 - DNA, 21 bp, artificial sequence
  synthetic aptamer
gguacggggagugugggcugg

SEQ ID NO: 191 - DNA, 28 bp, artificial sequence
  synthetic aptamer
ggcgguacggggggagugggcugggguc

SEQ ID NO: 192 - DNA, 27 bp, artificial sequence
  synthetic aptamer
ggcgguacggggggagugggcuggguc

SEQ ID NO: 193 - DNA, 28 bp, artificial sequence
  synthetic aptamer
ggcgguacggggagagugggcugggguc

SEQ ID NO: 194 - DNA, 22 bp, artificial sequence
  synthetic aptamer
gguacggggggagugggcuggg

SEQ ID NO: 195 - DNA, 21 bp, artificial sequence
  synthetic aptamer
gguacggggggagugggcugg

SEQ ID NO: 196 - DNA, 22 bp, artificial sequence
  synthetic aptamer
gguacggggagagugggcuggg

SEQ ID NO: 197 - DNA, 25 bp, artificial sequence
  synthetic aptamer
ggcgguacggggggagugggcuggg

SEQ ID NO: 198 - DNA, 25 bp, artificial sequence
  synthetic aptamer
ggcgguacggggaguguggguuggg

SEQ ID NO: 199 - RNA, 43 nt, artificial sequence
  synthetic aptamer
gggcacucagccacagguggcuuaauacuguaaagacgugccc

SEQ ID NO: 200 - RNA, 48 nt, artificial sequence
  synthetic aptamer
ggagcgcacucagccaccggcuuaauauccaauaggaacguucgcucu

SEQ ID NO: 201 - RNA, 48 nt, artificial sequence
  synthetic aptamer
gggcacucagccacagcucgguggcuuaauaucuaugugaacgugccc

SEQ ID NO: 202 - RNA, 43 nt, artificial sequence
  synthetic aptamer
gggcacucagccaccuugggcuuaauaccuaucggaugugccc

SEQ ID NO: 203 - DNA, 34 bp, artificial sequence; identical to SEQ ID NO: 177
  synthetic aptamer

SEQ ID NO: 204 - DNA, 34 bp, artificial sequence; identical to SEQ ID NO: 177
  synthetic aptamer

SEQ ID NO: 205 - DNA, 34 bp, artificial sequence; identical to SEQ ID NO: 177
  synthetic aptamer

SEQ ID NO: 206 - DNA, 34 bp, artificial sequence; identical to SEQ ID NO: 177
  synthetic aptamer

SEQ ID NO: 207 - DNA, 34 bp, artificial sequence; identical to SEQ ID NO: 177
  synthetic aptamer

SEQ ID NO: 208 - DNA, 34 bp, artificial sequence; identical to SEQ ID NO: 177
  synthetic aptamer

SEQ ID NO: 209 - DNA, 34 bp, artificial sequence; identical to SEQ ID NO: 177
  synthetic aptamer

SEQ ID NO: 210 - DNA, 34 bp, artificial sequence; identical to SEQ ID NO: 177
  synthetic aptamer

SEQ ID NO: 211 - DNA, 34 bp, artificial sequence; identical to SEQ ID NO: 177
  synthetic aptamer

SEQ ID NO: 212 - DNA, 34 bp, artificial sequence; identical to SEQ ID NO: 177
  synthetic aptamer

SEQ ID NO: 213 - DNA, 34 bp, artificial sequence; identical to SEQ ID NO: 177
  synthetic aptamer

SEQ ID NO: 214 - DNA, 34 bp, artificial sequence; identical to SEQ ID NO: 177
  synthetic aptamer

SEQ ID NO: 215 - DNA, 34 bp, artificial sequence; identical to SEQ ID NO: 177
  synthetic aptamer

SEQ ID NO: 216 - DNA, 34 bp, artificial sequence; identical to SEQ ID NO: 177
  synthetic aptamer

SEQ ID NO: 217 - DNA, 34 bp, artificial sequence; identical to SEQ ID NO: 177
  synthetic aptamer

SEQ ID NO: 218 - DNA, 34 bp, artificial sequence; identical to SEQ ID NO: 177
  synthetic aptamer

SEQ ID NO: 219 - DNA, 34 bp, artificial sequence; identical to SEQ ID NO: 177
  synthetic aptamer

SEQ ID NO: 220 - DNA, 34 bp, artificial sequence; identical to SEQ ID NO: 177
  synthetic aptamer

SEQ ID NO: 221 - DNA, 34 bp, artificial sequence; identical to SEQ ID NO: 177
  synthetic aptamer

SEQ ID NO: 222 - DNA, 34 bp, artificial sequence; identical to SEQ ID NO: 177
  synthetic aptamer

SEQ ID NO: 223 - DNA, 34 bp, artificial sequence; identical to SEQ ID NO: 177
  synthetic aptamer

SEQ ID NO: 224 - DNA, 35 bp, artificial sequence
  synthetic aptamer
acaggcaaguaauuggggagugcgggcggggugut

SEQ ID NO: 225 - DNA, 35 bp, artificial sequence; identical to SEQ ID NO: 224
  synthetic aptamer

SEQ ID NO: 226 - DNA, 35 bp, artificial sequence; identical to SEQ ID NO: 224
  synthetic aptamer

SEQ ID NO: 227 - DNA, 35 bp, artificial sequence; identical to SEQ ID NO: 224
  synthetic aptamer

SEQ ID NO: 228 - DNA, 35 bp, artificial sequence; identical to SEQ ID NO: 224
  synthetic aptamer

SEQ ID NO: 229 - DNA, 35 bp, artificial sequence; identical to SEQ ID NO: 224
  synthetic aptamer

SEQ ID NO: 230 - DNA, 35 bp, artificial sequence; identical to SEQ ID NO: 224
  synthetic aptamer

SEQ ID NO: 231 - DNA, 35 bp, artificial sequence; identical to SEQ ID NO: 224
  synthetic aptamer

SEQ ID NO: 232 - DNA, 35 bp, artificial sequence; identical to SEQ ID NO: 224
  synthetic aptamer

SEQ ID NO: 233 - DNA, 35 bp, artificial sequence; identical to SEQ ID NO: 224
  synthetic aptamer

SEQ ID NO: 234 - DNA, 35 bp, artificial sequence; identical to SEQ ID NO: 224
  synthetic aptamer

SEQ ID NO: 235 - DNA, 35 bp, artificial sequence; identical to SEQ ID NO: 224
  synthetic aptamer

SEQ ID NO: 236 - DNA, 35 bp, artificial sequence; identical to SEQ ID NO: 224
  synthetic aptamer

SEQ ID NO: 237 - DNA, 35 bp, artificial sequence; identical to SEQ ID NO: 224
  synthetic aptamer

SEQ ID NO: 238 - DNA, 35 bp, artificial sequence; identical to SEQ ID NO: 224
  synthetic aptamer

SEQ ID NO: 239 - DNA, 35 bp, artificial sequence; identical to SEQ ID NO: 224
  synthetic aptamer

SEQ ID NO: 240 - DNA, 35 bp, artificial sequence; identical to SEQ ID NO: 224
  synthetic aptamer

SEQ ID NO: 241 - DNA, 35 bp, artificial sequence; identical to SEQ ID NO: 224
  synthetic aptamer

SEQ ID NO: 242 - DNA, 35 bp, artificial sequence; identical to SEQ ID NO: 224
  synthetic aptamer

SEQ ID NO: 243 - DNA, 35 bp, artificial sequence; identical to SEQ ID NO: 224
  synthetic aptamer

SEQ ID NO: 244 - DNA, 35 bp, artificial sequence; identical to SEQ ID NO: 224
  synthetic aptamer

SEQ ID NO: 245 - DNA, 35 bp, artificial sequence; identical to SEQ ID NO: 224
  synthetic aptamer

SEQ ID NO: 246 - DNA, 35 bp, artificial sequence; identical to SEQ ID NO: 224
  synthetic aptamer

SEQ ID NO: 247 - DNA, 35 bp, artificial sequence; identical to SEQ ID NO: 224
  synthetic aptamer

SEQ ID NO: 248 - DNA, 35 bp, artificial sequence; identical to SEQ ID NO: 224
  synthetic aptamer

SEQ ID NO: 249 - DNA, 35 bp, artificial sequence; identical to SEQ ID NO: 224
  synthetic aptamer

SEQ ID NO: 250 - DNA, 35 bp, artificial sequence; identical to SEQ ID NO: 224
  synthetic aptamer

SEQ ID NO: 251 - DNA, 35 bp, artificial sequence; identical to SEQ ID NO: 224
  synthetic aptamer

SEQ ID NO: 252 - DNA, 35 bp, artificial sequence; identical to SEQ ID NO: 224
  synthetic aptamer

SEQ ID NO: 253 - DNA, 35 bp, artificial sequence; identical to SEQ ID NO: 224
  synthetic aptamer

SEQ ID NO: 254 - DNA, 35 bp, artificial sequence; identical to SEQ ID NO: 224
  synthetic aptamer

SEQ ID NO: 255 - DNA, 35 bp, artificial sequence; identical to SEQ ID NO: 224
  synthetic aptamer

SEQ ID NO: 256 - DNA, 35 bp, artificial sequence; identical to SEQ ID NO: 224
  synthetic aptamer

SEQ ID NO: 257 - DNA, 35 bp, artificial sequence; identical to SEQ ID NO: 224
  synthetic aptamer

SEQ ID NO: 258 - DNA, 35 bp, artificial sequence; identical to SEQ ID NO: 224
  synthetic aptamer

SEQ ID NO: 259 - DNA, 35 bp, artificial sequence; identical to SEQ ID NO: 224
  synthetic aptamer

SEQ ID NO: 260 - DNA, 35 bp, artificial sequence; identical to SEQ ID NO: 224
  synthetic aptamer

SEQ ID NO: 261 - DNA, 35 bp, artificial sequence; identical to SEQ ID NO: 224
  synthetic aptamer

SEQ ID NO: 262 - DNA, 35 bp, artificial sequence; identical to SEQ ID NO: 224
  synthetic aptamer

SEQ ID NO: 263 - DNA, 35 bp, artificial sequence; identical to SEQ ID NO: 224
  synthetic aptamer

SEQ ID NO: 264 - DNA, 35 bp, artificial sequence; identical to SEQ ID NO: 224
  synthetic aptamer

SEQ ID NO: 265 - DNA, 37 bp, artificial sequence
  synthetic aptamer
cgcaggcaaguaauuggggagugcgggcggggugcgt

SEQ ID NO: 266 - DNA, 29 bp, artificial sequence
  synthetic aptamer
ggcaaguaauuggggagugcgggcggggt

SEQ ID NO: 267 - DNA, 29 bp, artificial sequence
  synthetic aptamer
ggcaaguaauuggggagugcgggcggggt

SEQ ID NO: 268 - DNA, 29 bp, artificial sequence
  synthetic aptamer
ggcaaguaauuggggagugcgggcggggt

SEQ ID NO: 269 - DNA, 29 bp, artificial sequence
  synthetic aptamer
ggcaaguaauuggggagugcgggcggggt

SEQ ID NO: 270 - DNA, 35 bp, artificial sequence; identical to SEQ ID NO: 224
  synthetic aptamer

SEQ ID NO: 271 - DNA, 35 bp, artificial sequence; identical to SEQ ID NO: 224
  synthetic aptamer

SEQ ID NO: 272 - DNA, 35 bp, artificial sequence; identical to SEQ ID NO: 224
  synthetic aptamer

SEQ ID NO: 273 - DNA, 35 bp, artificial sequence; identical to SEQ ID NO: 224
  synthetic aptamer

SEQ ID NO: 274 - DNA, 35 bp, artificial sequence; identical to SEQ ID NO: 224
  synthetic aptamer

SEQ ID NO: 275 - DNA, 35 bp, artificial sequence; identical to SEQ ID NO: 224
  synthetic aptamer

SEQ ID NO: 276 - DNA, 35 bp, artificial sequence; identical to SEQ ID NO: 224
  synthetic aptamer

SEQ ID NO: 277 - DNA, 35 bp, artificial sequence
  synthetic aptamer
acangcaaguaauuggggagugcgggcggggugut

SEQ ID NO: 278 - DNA, 35 bp, artificial sequence
  synthetic aptamer
acagncaaguaauuggggagugcgggcggggugut

SEQ ID NO: 279 - DNA, 35 bp, artificial sequence
  synthetic aptamer
acanncaaguaauuggggagugcgggcggggugut

SEQ ID NO: 280 - DNA, 35 bp, artificial sequence
  synthetic aptamer
acaggcaanuaauuggggagugcgggcggggugut

SEQ ID NO: 281 - DNA, 35 bp, artificial sequence
  synthetic aptamer
acaggcaaguaauungggagugcgggcggggugut

SEQ ID NO: 282 - DNA, 35 bp, artificial sequence
  synthetic aptamer
acaggcaaguaauugnggagugcgggcggggugut

SEQ ID NO: 283 - DNA, 35 bp, artificial sequence
  synthetic aptamer
acaggcaaguaauuggngagugcgggcggggugut

SEQ ID NO: 284 - DNA, 35 bp, artificial sequence
  synthetic aptamer
acaggcaaguaauugggnagugcgggcggggugut

SEQ ID NO: 285 - DNA, 35 bp, artificial sequence
  synthetic aptamer
acaggcaaguaauunnggagugcgggcggggugut

SEQ ID NO: 286 - DNA, 35 bp, artificial sequence
  synthetic aptamer
acaggcaaguaauugnngagugcgggcggggugut

SEQ ID NO: 287 - DNA, 35 bp, artificial sequence
  synthetic aptamer
acaggcaaguaauuggnnagugcgggcggggugut

SEQ ID NO: 288 - DNA, 35 bp, artificial sequence
  synthetic aptamer
acaggcaaguaauunnnnagugcgggcggggugut

SEQ ID NO: 289 - DNA, 35 bp, artificial sequence
  synthetic aptamer
acaggcaaguaauugggganugcgggcggggugut

SEQ ID NO: 290 - DNA, 35 bp, artificial sequence
  synthetic aptamer
acaggcaaguaauuggggaguncgggcggggugut

SEQ ID NO: 291 - DNA, 35 bp, artificial sequence
  synthetic aptamer
acaggcaaguaauuggggagugcnggcggggugut

SEQ ID NO: 292 - DNA, 35 bp, artificial sequence
  synthetic aptamer
acaggcaaguaauuggggagugcgngcggggugut

SEQ ID NO: 293 - DNA, 35 bp, artificial sequence
  synthetic aptamer
acaggcaaguaauuggggagugcggncggggugut

SEQ ID NO: 294 - DNA, 35 bp, artificial sequence
  synthetic aptamer
acaggcaaguaauuggggagugcnngcggggugut

SEQ ID NO: 295 - DNA, 35 bp, artificial sequence
  synthetic aptamer
acaggcaaguaauuggggagugcgnncggggugut

SEQ ID NO: 296 - DNA, 35 bp, artificial sequence
  synthetic aptamer
acaggcaaguaauuggggagugcnnncggggugut

SEQ ID NO: 297 - DNA, 35 bp, artificial sequence
  synthetic aptamer
acaggcaaguaauuggggagugcgggcngggugut

SEQ ID NO: 298 - DNA, 35 bp, artificial sequence
  synthetic aptamer
acaggcaaguaauuggggagugcgggcgnggugut

SEQ ID NO: 299 - DNA, 35 bp, artificial sequence
  synthetic aptamer
acaggcaaguaauuggggagugcgggcggngugut

SEQ ID NO: 300 - DNA, 35 bp, artificial sequence
  synthetic aptamer
acaggcaaguaauuggggagugcgggcgggnugut

SEQ ID NO: 301 - DNA, 35 bp, artificial sequence
  synthetic aptamer
acaggcaaguaauuggggagugcgggcnnggugut

SEQ ID NO: 302 - DNA, 35 bp, artificial sequence
  synthetic aptamer
acaggcaaguaauuggggagugcgggcgnngugut

SEQ ID NO: 303 - DNA, 35 bp, artificial sequence
  synthetic aptamer
acaggcaaguaauuggggagugcgggcggnnugut

SEQ ID NO: 304 - DNA, 35 bp, artificial sequence
  synthetic aptamer
acaggcaaguaauuggggagugcgggcnnnnugut

SEQ ID NO: 305 - DNA, 35 bp, artificial sequence
  synthetic aptamer
acaggcaaguaauuggggagugcgggcggggunut

SEQ ID NO: 306 - DNA, 35 bp, artificial sequence; identical to SEQ ID NO: 224
  synthetic aptamer

SEQ ID NO: 307 - DNA, 35 bp, artificial sequence; identical to SEQ ID NO: 224
  synthetic aptamer

SEQ ID NO: 308 - DNA, 35 bp, artificial sequence; identical to SEQ ID NO: 224
  synthetic aptamer

SEQ ID NO: 309 - DNA, 35 bp, artificial sequence; identical to SEQ ID NO: 224
  synthetic aptamer

SEQ ID NO: 310 - DNA, 35 bp, artificial sequence; identical to SEQ ID NO: 224
  synthetic aptamer

SEQ ID NO: 311 - DNA, 35 bp, artificial sequence; identical to SEQ ID NO: 224
  synthetic aptamer

SEQ ID NO: 312 - DNA, 35 bp, artificial sequence; identical to SEQ ID NO: 224
  synthetic aptamer

SEQ ID NO: 313 - DNA, 35 bp, artificial sequence; identical to SEQ ID NO: 224
  synthetic aptamer

SEQ ID NO: 314 - DNA, 35 bp, artificial sequence; identical to SEQ ID NO: 224
  synthetic aptamer

SEQ ID NO: 315 - protein, 335 aa, Mus musculus
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 316 - protein, 196 aa, mus musculus
MLDCRAVIMLWLLPWVTQGLAVPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTA

SEQ ID NO: 317 - protein, 215 aa, mus musculus
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA